(19) United States
(12) Patent Application Publication
(10) Pub. No.: US 2012/0129957 A1
(43) Pub. Date: May 24, 2012

(54) ANALYZING THE FMR1 GENE

(76) Inventors: Norbert GLEICHER, Chicago, IL (US); David H. BARAD, Closter, NJ (US)

(21) Appl. No.: 13/360,349

(22) Filed: Jan. 27, 2012

Related U.S. Application Data
(63) Continuation-in-part of application No. 13/043,199, filed on Mar. 8, 2011.
Application No. 13/043,199 is a continuation-in-part of application No. 12/508,295, filed on Jul. 23, 2009.

(51) Int. Cl. A61K 47/46; A61P 35/00; C12Q 1/68

(52) U.S. Cl. 514/789; 435/6.12

(57) ABSTRACT

A method of screening a human for risk of malignancies is disclosed. The method may include isolating the human's FMR1 gene, wherein the FMR1 gene has a first allele and a second allele, measuring the number of triple CGG repeats on each of the first and second alleles, wherein the measuring step is conducted through use of an assay, and identifying the human as at risk for cancer when the triple CGG repeat number for at least one of the first and second alleles is less than 26.

### CROSS REFERENCE TO RELATED APPLICATIONS

This application is a continuation-in-part of applications Ser. No. 13/043,199, filed Mar. 8, 2011 and Ser. No. 12/508,295, filed on Jul. 23, 2009, which are incorporated by reference in their entirety.

### BACKGROUND OF THE INVENTION

1. Field of the Invention

The present invention relates to a method of determining ovarian function, ovarian reserve, pregnancy chances, risk of autoimmunity, and risk of cancer in a female by evaluating CGG repeats on the FMR1 gene.

2. Description of the Related Art

A dynamic triple-repeat sequence mutation in the X-linked gene, known as FMR1 (fragile X mental retardation 1), in its fully expanded form encompassing over 200 hypermethylated expansions of CGG, and expanding to the gene's promoter region, represents the full mutation for the so-called fragile X syndrome. Once the molecular biology of the syndrome was understood, it became apparent that between normal (or common) findings and full mutation, two additional stages of expansion exist, the so-called gray (or intermediate) zone and so-called premutations. There is consensus that premutations involve 55 to 200 repeats, and full mutations involve over 200 repeats. Whether the intermediate zone starts at 40 or 45 repeats has remained controversial, though, excluding American College of Obstetrics and Gynecology (ACOG) criteria, consensus is that the intermediate zone extends to 54 repeats.

Completely unaffected individuals most frequently demonstrate between 26 and 34 repeats with a median of approximately 30. Most laboratories, however, consider anything under 45 repeats a negative result. This may require reevaluation; because studies suggest that premature ovarian failure (POF) may be increased at intermediate-size alleles of approximately 41 to 58 repeats. Experts, recently summarizing the state of the art after two federally funded consensus meetings, concluded that more data was needed to confirm this latter association. The ACOG considers a patient unaffected with ≦40 repeats, intermediate with 41-60, and a premutation between 61 and 200 repeats.

Carriers of premutations do not suffer from classical symptoms of fragile X, such as mental retardation. Their alleles, however, are in subsequent generations at significant risk of further expansion to the fully developed mutation. Carriers are, nevertheless, phenotypically affected: males with premutations are at increased risk for the so-called fragile X-associated tremor/ataxia syndrome (FXTAS), a progressive neurodegenerative disorder, whereas affected women have only a very low risk for FXTAS, but experience a high prevalence of premature ovarian failure (POF).

True POF represents an end stage of ovarian function. In many instances it is reached quickly and without preceding symptoms and/or laboratory abnormalities. In other cases, it may be preceded by lengthy periods of clinically symptomatic diminished ovarian reserve. When young women demonstrate symptoms of diminished ovarian reserve, such as age-specific elevated baseline follicle stimulating hormone (FSH) levels and/or ovarian resistance to stimulation with gonadotropins, an acronym of premature ovarian aging (POA) has been coined to differentiate these clinical circumstances from end-stage POF patients and women with ovarian senescence because of advanced age.

Such differentiation is important because, in contrast to POF, POA patients still demonstrate a fair chance of pregnancy with autologous oocytes up to b-FSH levels of approximately 40 mIU/mL, and therefore, represent a milder degree of ovarian dysfunction than POF. Whether POA patients share the underlying pathophysiologies and etiologies of POF is unknown. It seems reasonable, however, to assume that at least some POA patients will transition into POF, and that therefore, at least in these patients, POA may represent a continuum of impaired ovarian function.

We have previously pointed out that abnormal autoimmune function is frequently overlooked, and may be underappreciated as a cause of female infertility. Improvement in a diagnosis between the FMR1 gene and ovarian function is needed to determine whether a woman has risk towards autoimmunity and determine her pregnancy chances to accelerate appropriate clinical interventions.

Furthermore, both the FMR1 gene and the BRCA genes may effect ovarian reserve. FMR1 also may impact pregnancy chances in association with in vitro fertilization and define risk towards autoimmunity, but the ovarian effects of BRCA genes are unclear. A better understanding of the ovarian effects of the BRCA genes may lead to improvement in screening and may accelerate appropriate and valuable clinical interventions and/or treatment.

Moreover, for decades, androgens have been considered detrimental to follicle maturation. Some animal studies now suggest that they are essential for normal folliculogenesis. Therefore, it is important to understand the endocrinology of androgen supplementation in women, and its effects on pregnancy potential in women with diminished ovarian reserve, and whether these effects change in association with FMR1-genotypes. Improvement in determining the effectiveness of androgen supplementation and a woman's pregnancy potential will facilitate and accelerate appropriate clinical interventions and/or treatment.

### BRIEF SUMMARY OF THE INVENTION

A method of predicting a degree of risk of autoimmunity in a human female is disclosed. The method may include analyzing the female's FMR1 gene, wherein the FMR1 gene has a first allele and a second allele, determining the number of triple CGG repeats on each of the first and second alleles; defining a normal range of triple CGG repeats; and comparing the number of triple CGG repeats on each of the first and second alleles to the normal range. If the triple CGG repeat number for one of the first and second alleles is in the normal range and the triple CGG repeat number for the other one of the first and second alleles is less than the lower boundary of the normal range, then the female is at increased risk of autoimmunity.

A method of predicting a degree of risk of autoimmunity may define the normal range between 26 and 34 triple CGG repeats with respect to autoimmunity, wherein 26 is the lower boundary and 34 is the upper boundary.

Also, the method of predicting a degree of risk of autoimmunity may further include that the human female may be at a further increased risk of autoimmunity if the human female has a polycystic ovary-like phenotype. The human female may be considered to have a polycystic ovary-like phenotype if the human female has an anti-Müllerian hormone level above about 4.0 ng/mL.

A method of predicting a degree of risk of autoimmunity further may include that if the triple CGG repeat number for one of the first and second alleles is outside of the normal range and the triple CGG repeat number for the other one of the first and second alleles is within the normal range, then the first and second alleles are heterozygous.

A method of predicting a degree of risk of autoimmunity further may include analyzing at least one of the human female's follicle stimulating hormone level and the human female's anti-Müllerian hormone level.

A method of predicting a degree of risk of autoimmunity further may include that if the triple CGG repeat numbers for both of the first and second alleles are outside of the normal range, then the first and second alleles are homozygous and the female is at a decreased risk of autoimmunity.

A method of predicting a degree of risk of autoimmunity also may include that if the triple CGG repeat number for one of the first and second alleles is in the normal range and the triple CGG repeat number for the other one of the first and second alleles is greater than the upper boundary of the normal range, then the female is at a further decreased risk of autoimmunity.

A method of predicting a degree of risk of autoimmunity further may include that if the triple CGG repeat number for one of the first and second alleles is in the normal range and the triple CGG repeat number for the other one of the first and second alleles is greater than the upper boundary of the normal range, then the female is protected from autoimmunity.

In another aspect, a method of predicting pregnancy chances for a human female is disclosed. The method may include analyzing the female's FMR1 gene, wherein the FMR1 gene has a first allele and a second allele; determining the number of triple CGG repeats on each of the first and second alleles; defining a normal range of triple CGG repeats; and comparing the number of triple CGG repeats on each of the first and second alleles to the normal range. If the triple CGG repeat number for one of the first and second alleles is in the normal range and the triple CGG repeat number for the other one of the first and second alleles is less than the lower boundary of the normal range, then the female has decreased chances of pregnancy.

The method of predicting pregnancy chances may define the normal range between 26 and 34 triple CGG repeats with respect to pregnancy chances, wherein 26 is the lower boundary and 34 is the upper boundary.

Also, the method of predicting pregnancy chances further may include that the chances of pregnancy are with respect to in-vitro fertilization.

The method of predicting pregnancy chances further may include that if the triple CGG repeat numbers for one of the first and second alleles is less than the lower boundary of the normal range and the triple CGG repeat numbers for the other one of the first and second alleles is within the normal range, then the first and second alleles are heterozygous.

The method of predicting pregnancy chances also may include that if the triple CGG repeat numbers for one of the first and second alleles is greater than the upper boundary of the normal range and the triple CGG repeat numbers for the other one of the first and second alleles is within the normal range, then the first and second alleles are heterozygous and the female has a greater or increased chance of achieving pregnancy, then if the triple CGG repeat number for one of the first and second alleles is the normal range and the other one of the first and second alleles is less than the lower boundary of the normal range.

The method of predicting pregnancy chances also may include that if the triple CGG repeat numbers for both of the first and second alleles are within the normal range, then the first and second alleles are normal and the female has an even greater chance of achieving pregnancy.

A method of screening a human for risk of malignancies is disclosed. The method may include isolating the human's FMR1 gene, wherein the FMR1 gene has a first allele and a second allele, measuring the number of triple CGG repeats on each of the first and second alleles, wherein the measuring step is conducted through use of an assay, and identifying the human as at risk for cancer when the triple CGG repeat number for at least one of the first and second alleles is less than 26.

A method of screening a human female for the clinical effectiveness of an androgen in a human female is disclosed. The method may include measuring the female's testosterone levels, administering an androgen to the female, isolating the female's FMR1 gene, wherein the FMR1 gene has a first allele and a second allele, measuring the number of triple CGG repeats on each of the first and second alleles, wherein the measuring step is conducted through use of an assay, measuring the female's testosterone levels again, identifying the androgen as clinically effective such that the female has an increased pregnancy potential when the female's testosterone levels were higher when measured again and when the triple CGG repeat number for at least one of the first and second alleles is less than 26 or greater than 34.

A method of screening a human for increased embryo quality is disclosed. The method may include isolating at least one of the human's BRC1 and BRC2 genes, analyzing each of the BRC1 and BRC2 genes for mutations, isolating the human's FMR1 gene, wherein the FMR1 gene has a first allele and a second allele, when a mutation of at least one of the BRC1 and BRC2 gene is present, measuring the number of triple CGG repeats on each of the first and second alleles, wherein the measuring step is conducted through use of an assay, and identifying increased embryo quality and increased embryo survival, when the triple CGG repeat number for at least one of the first and second alleles is less than 26.

### BRIEF DESCRIPTION OF THE DRAWINGS

FIG. 1 is a graph showing the distribution of CGG repeat counts.

FIG. 2A is a graph showing a correlation between number of triple CGG repeats on allele-2 and oocyte yield for individuals <38 years of age.

FIG. 2B is a graph showing a correlation between number of triple CGG repeats on allele-2 and oocyte yield for individuals ≧38 years of age.

FIG. 3 is a graph showing distribution of CGG triple repeat expansions on the FMR1 in the whole study population of infertile women compared with a general population.

FIG. 4 is a scattergram of AMH levels in correlation to triple CGG repeats on allele-2.

FIG. 5 is a graph showing mean CGG triple repeats in correlation to AMH levels at different ages.

FIG. 6 is a graph showing correlation between triple CGG repeats on the FMR1 gene with AMH levels in range between 35-50 CGG repeat (Group 2).

FIG. 7 is a box and whisker plot of triple CGG counts for an entire patient population.

FIG. 8 is a box and whisker plots of triple CGG counts for individual racial/ethnic groups.

FIG. 9 is a box and whisker plot that defines the normal range of CGG repeats and FIG. 9A is a graph showing CGG repeats in correlation to Relative Frequency.

FIG. 10A is a box and whisker plot that defines the normal AMH levels and outliers for egg donors.

FIG. 10B is a box and whisker plot that defines the normal AMH levels and outliers for infertility patients.

FIG. 11 is a linear regression of association between AMH levels and age based on FMR1 status.

FIG. 12 is a graph showing AMH levels in binned age groups based on FMR1 status.

FIG. 12A.1 is a graph showing age specific AMH levels.

FIG. 12A.2 is a graph showing age specific FSH levels.

FIG. 12B is a graph showing the distribution of CGG repeats on FMR1 gene in candidates.

FIG. 13 is a graph showing the prevalence of autoimmunity in reference to FMR1 genotype.

FIG. 14 is a graph showing pregnancy rates in IVF based on FMR1 genotype.

FIG. 15 is a logical regression of DOR (low AMH) and CGG counts, adjusted for age.

FIG. 16 is a linear regression of AHM levels, depending on genotypes normal (norm), heterozygous (het) and homozygous (hom).

FIG. 17 is a graph showing the distribution of FMR1 genotypes and sub-genotypes in women with BRC1/2 mutations and U.S. comparison group.

FIG. 18A is a graph showing the distribution of FMR1 alleles in BRCA1/2 carriers, and the horizontal and vertical parallel lines define the normal distribution area.

FIG. 18B is a graph showing the distribution of FMR1 alleles in the fertility controls, and the horizontal and vertical parallel lines define the normal distribution area.

FIG. 19A is a graph showing the distribution of FMR1 genotypes and sub-genotypes in women with BRC1/2 mutations and U.S. comparison group.

FIG. 19B is a graph showing the distribution of FMR1 genotypes and sub-genotypes in women with BRC1/2 mutations and U.S. comparison group.

FIGS. 20 a-d are longitudinal androgen concentrations (monthly intervals) and pregnancy chance in 91 women with diminished ovarian reserve, who underwent DHEA supplementation and consecutive in vitro fertilization (IVF).

FIGS. 21a-h are longitudinal androgen concentrations (monthly intervals) and pregnancy chances in 91 women with diminished ovarian reserve, who underwent DHEA supplementation and consecutive in vitro fertilization (IVF) stratified for FMR1 genotypes.

### DETAILED DESCRIPTION OF THE INVENTION

We disclose our discovery of new, previously totally unknown genotypes and sub-genotypes of the FMR1 gene, which have distinct diagnostic screening and/or testing functions. The genotypes and sub-genotypes are based on our recognition of a normal CGG triple repeat range of approximately 26-34, previously unknown. Up until we discovered this range, everything under 45 CGG repeats had been considered normal, 45 and above was considered abnormal, with 45-55 considered intermediate, 55-200 pre-mutation, and over 200 full mutation. But even these “abnormals” have nothing to do with the diagnostic features characterized by the genotypes and sub-genotypes we describe herein.

Our genotypes and sub-genotypes statistically associate with specific clinical diagnoses/risks in 3 distinct medical areas: (i) infertility; (ii) autoimmunity; and (iii) cancer. The details are disclosed herein.

Other associations can be expected in the future. Therefore, these genotypes and sub-genotypes are new, based on the newly described normal range of (approximately) 26-34 triple CGG repeats.

Current medical knowledge neither allows for the prospective evaluation of ovarian function in young women, nor the accurate assessment of current ovarian function in infertile patients. A new test to predict ovarian function and assess female infertility is needed. The availability of a more objective measure of ovarian function would, therefore, improve the care of female infertility patients to a major degree.

As such, a new function for the FMR1 gene is defined herein (as opposed to the current function for the FMR1 gene that is only considered to affect neuropsychiatric issues). Specifically, the FMR1 gene affects ovarian function by regulating the amount of immature follicles released from ovarian storage, which a woman is born with, and, thereby, determining the “ovarian aging” process, which is a reflection of how many eggs still remain within the ovaries. The ability of the FMR1 gene allows us, based on CGG numbers, to predict whether someone is at risk for early ovarian aging.

Moreover, testing for triple-repeat sequence mutation in the FMR1 gene currently is carried out in four groups of patients. The first group includes boys showing characteristics of fragile X mental retardation. The second group includes middle-aged men showing symptoms of the so-called fragile X-associated tremor/ataxia syndrome (FXTAS). The third group includes pregnant woman, to determine the risk of fragile X mental retardation in their unborn child. The fourth group includes a very small proportion of woman considering becoming pregnant, again to determine the risk of fragile X mental retardation in their yet-to-be conceived child. Other groups of patients may benefit from analyzing the FRM1 gene with respect to ovarian function/reserve as disclosed herein.

### Pilot Studies

The fragile X mental retardation protein has so far not been well investigated in its effect on ovarian function. As such, we conducted a first pilot study that reports on the correlation between FMR1 testing and ovarian reserve parameters in infertile women, concluding that the number of triple CGG repeats of the FMR1 gene appears to be reflective of ovarian reserve.

After demonstrating that ranges starting at ≧30 repeats may represent a correlation between triple repeat numbers and risk for premature decline in ovarian function in the first pilot test, we conducted further pilot testing. Specifically, considering the correlation between number of CGG triple repeats on the FMR1 gene and risk for progressively serious forms of premature ovarian senescence, and the causal relationship between abnormal autoimmune function and premature ovarian senescence, the question arises as to whether these two etiologies represent independent risks. We, therefore, investigated in an infertile female population the relationship between triple repeat numbers on the FMR1 gene and abnormal autoimmune function.

The second pilot study found that twenty-two of 40 patients (55%) demonstrated autoimmune abnormalities. Women with and without autoimmune abnormalities did not differ in age. The study demonstrated that the designation of patients as autoimmune may separate into two distinct patient populations with entirely different genotype (number of triple repeats on the FMR1 gene) and phenotype (FSH and AMH levels). Again, in view of the small study size and lack of specific markers for “abnormal autoimmune function,” the data discussed above should be considered preliminary and requires further confirmation. The second pilot study observations deserve further exploration, but abnormal autoimmune function and expansions ≧30 CGG triple repeats seem to reflect independent risks toward premature ovarian senescence. How the fact that both of these etiologies adversely affect ovarian function and, independently, lead toward POA remained to be determined. As discussed herein, a study, with a larger patient population, discusses the FMR1 gene and autoimmunity.

### Groups of Patients and Home Testing

Different set of patients may benefit from analyzing the FRM1 gene as disclosed herein. A first set includes women with a diagnosis of infertility. This set includes women who have been unsuccessful in conceiving after 6-12 months of attempting to become pregnant. Testing for mutation in the FMR1 gene may be used to determine the etiology of the infertility. Infertility in most women results from a mutation in the FMR1 gene (genetic etiology) or results from autoimmunity (autoimmune etiology). Over ⅔ of all premature ovarian aging can be determined with evidence from the FMR1 gene and evidence of (even subclinical, i.e., only laboratory detectable) autoimmunity. Such testing may be coupled with a determination of autoimmunity, for example by looking for symptoms of other existing autoimmune disorders, such as psoriasis, lupus, etc, or actual testing for autoimmune antibodies.

A second set includes women who are not considering becoming pregnant, and who are deciding to delay having children. Women in this age set may be, for example 18-40 years old, more preferably 18-35 years old, including women at most 30 years old, especially 18-25 years old, including 18-23 and 18-22 years old. Also included may be women 25-40 years old, including 25-35 years old, and 25-30 years old. About 10% of the general population will have a mutation in the FMR1 gene. Testing for this mutation, and the extent of the triple-repeat sequence mutation, will determine if a woman in this set is at risk for POA. Such testing may be coupled with a determination of autoimmunity, for example by looking for symptoms of other existing autoimmune disorders, such as psoriasis, lupus, etc, or actual testing for autoimmune antibodies. If a mutation is found, then action may be taken to monitor ovarian function, such as subsequent regular testing for AMH; this is particularly useful in those patient at least 25 years old (for example women 25-40 years old, including 25-35 years old, and 25-30 years old), as AMH levels will typically not indicate POA until the mid-twenties. Alternatively, or in addition, steps may be taken to address the risk of POA, for example by collecting and freezing eggs of the patient.

Many women in this second set may show no symptoms, and may not visit a doctor because they are not considering becoming pregnant; rather they are seeking to delay reproduction. Therefore, a kit for home collection of a DNA sample, together with preaddressed and optionally prepaid mailer, would allow such women to be tested for mutations in the FMR1 gene. Collection of a DNA sample may be done by sampling any one of a variety of tissues, with a tissue sampler, including skin from inside the mouth, such as a mouth swab, or a small blood sample, for example a lance prick of the finger or the arm, and the blood may be soaked into a small piece of absorbent paper, or a cotton swab. The kit may also include a questionnaire for determination of autoimmunity, for example by having questions regarding symptoms of autoimmune disorders. The DNA sample may be sent to a testing facility, such as a laboratory, to test the DNA sample for mutation in the FMR1 gene. The results of the test may then be provided directly to the patient, for example by results printed out and sent back to the patient, by electronic mail (e-mail), by telephone, or the patient may be directed to a website. Alternatively, results may be sent to a physician, such as a physician designated by the patient, or a physician designated by the supplier of the kit, either from a list, or a physician close to the patient selected via a website or by telephone, who can provide the test results to the patient. Instruction may be included in the kit, in particular instructions for collection of the DNA sample, and optionally instruction regarding a questionnaire for determination of autoimmunity, or instruction regarding the results of the test, such as instructions for accessing the patient specific results through a website. The instructions may further include the intended use of the kit and/or test, and the instructions may state the ideal range of results. For example, the instructions may state that if a female's CGG repeats are within a normal or ideal range of 26-34 repeats with regard to ovarian function/reserve, then there is no indication of an increased risk for diminished ovarian aging or an increased risk or autoimmunity or an increased risk of cancer. Further, the instructions may explain the meaning of having CGG repeats within the normal range and/or action steps if the results are outside the ideal range. For example, if the CGG repeats are outside the normal range of 26-34 repeats, then the instructions may instruct the female to consult with a doctor for further evaluation, analysis and/or testing. For example, further testing may include tests for diminished ovarian reserve and/or for AMH levels, or may include BRC1/BRC2 analysis and testing.

A method of using a kit is disclosed. The kit may include a tissue sampler, a mailer preaddressed for a laboratory, and instructions comprising directing collection of tissue from a female considering delaying reproduction. The method may include collecting the tissue following the instructions, mailing the tissue to a laboratory for evaluation, receiving results from the laboratory, wherein the results include the number of CGG repeats on each allele of the female, and evaluating the results. Evaluating the results may include comparing the number of CGG repeats on each allele to a normal range of 26-34, and determining whether the number of CGG repeats are within the normal range, and if either number of CGG repeats is outside of the normal range, then the female is at an increased risk for early ovarian aging. Similarly, evaluating the results may include comparing the number of CGG repeats on each allele to a normal range of 26-34, and determining whether the number of CGG repeats are within the normal range, and if at least one of the numbers of CGG repeats is less than 26, then the female is at an increased risk for early ovarian aging, autoimmunity and/or cancer.

### A Study of FMR1 Gene and Abnormal Autoimmunity As Independent Risk Factors

The preliminary pilot studies suggested that, based on number of triple trinucleate (CGG) repeats (genetic POA) and immune laboratory abnormalities (autoimmune POA), women with POA, like those with POF, can be phenotypically and genotypically separated, combined representing a majority of patients with POA. These studies also suggested a functional correlation between triple CGG repeat numbers and ovarian function. However, because of small patient numbers and some outliers at very high repeats (full mutations), these studies lacked a satisfactory degree of statistical significance, and were therefore designated as pilot studies.

The risk for premature ovarian failure (POF) increases in association with two principal known etiologies: in the presence of excessive triple CGG expansions on the FMR1 (fragile X) gene and in association with a variety of autoimmune conditions. Our objective in this study was to determine to what degree milder forms of premature ovarian aging are also associated with these two etiologies (i.e., genetic etiology, autoimmune etiology). See at least Example 1 below.

We, therefore, investigated 119 consecutive, so identified, infertile women and statistically correlated by linear and logistic regression analyses ovarian function parameters to markers of a possible genetic etiology (number of CGG triple repeats on the FMR1 gene), and to markers of possible abnormal immune function (immune panel).

Our results were that sixty of 119 patients (50.4%) demonstrated at least one immune abnormality. Both groups did not differ statistically in age, mean follicle stimulating hormone (FSH), estradiol and anti-Müllerian hormone (AMH) levels, but AMH suggested a trend towards higher levels in autoimmune patients (p=0.19). Further, autoimmune patients also demonstrated lower mean triple CGG expansion sizes (p<0.05) and included fewer women with ≧35 triple repeats (RR 4.0, 1.3-11.9; p<0.01), previously reported to demarcate increased risk for premature ovarian aging.

Based on our results, we conclude that even minimal evidence of abnormal autoimmune function (“immunological noise”) increases risk towards premature ovarian aging, often manifesting as infertility. Evidence of abnormal autoimmune function, like increased CGG triple expansion sizes, in young women, therefore, warrants vigilance towards development of prematurely diminished ovarian reserve and infertility.

### A Study of FMR1 Gene and Ovarian Stimulation

In another study, we continued our testing and analyzing of CGG repeat counts on the FMR1 gene to determine whether the FMR1 gene can serve as a predictor of response to ovarian stimulation.

Abnormally large triple CGG repeat counts on the FMR1 gene, especially at the high end of currently considered normal range (gray zone) and at premutation range, have been associated with increased risk towards the most severe form of premature ovarian senescence, POF. We recently expanded on this association by demonstrating a direct statistical association between number of triple CGG counts (at 35 to 55 repeats) and ovarian reserve, as reflected by anti-Müllerian hormone (AMH) levels. The higher the count, the higher the risk for premature ovarian senescence and severe ovarian compromise. These observations suggest that the number of triple CGG repeats on the FMR1 gene may represent useful additional diagnostic tests in the evaluation of female infertility.

A concept, therefore, emerges, which suggests that, at younger age, normal future ovarian function appears assured (baring other etiologies adversely affecting ovarian reserve) with triple repeat counts below approximately 35, while higher counts reflect risk towards premature ovarian senescence. Older age, of course, is automatically associated with increasing ovarian senescence and the premature occurrence of ovarian aging may, therefore, clinically be less obvious.

Fu et al reported that the general population demonstrates a prominent distribution peak between 29 and 30 triple repeats. Thirty repeats have been suggested as the switching point between positive and negative translation effects of the FMR1 gene product. CGG counts above this apparently “normal” range, therefore, have potential clinical significance at two levels: they may be predictive of future risk towards premature ovarian senescence in young women, but may also help in assessing current risk towards diminished ovarian reserve and, thus, potentially serve as a standard ovarian function test in fertility therapy.

Based on the prevalence of early menopause, premature ovarian senescence has been suggested to occur in approximately 10 percent of all females. Early ovarian senescence, of course, is associated with an increased probability of female infertility. Not surprisingly, women under infertility treatment, therefore, demonstrate an even higher prevalence.

The potential value of triple CGG counts in reference to specific fertility parameters has, however, remained undefined. In this additional study, we, therefore, investigated whether triple CGG repeat counts on the FMR1 gene have predictive value for the ovarian response to stimulation with gonadotropins and oocyte yield during in vitro fertilization (IVF). In other words, whether triple CGG repeat counts on the FMR1 gene can serve as a diagnostic tool in fertility practice. See at least Example 2 below.

Since triple CGG repeats on FMR1 correlate with anti-Müllerian hormone (AMH), CGG repeats may also correlate with clinical outcomes. In 55 in vitro fertilization (IVF) patients, repeats, corrected for gonadotropin dosage, were, therefore, correlated to oocytes. Patients were stratified by <35 and ≧35 repeats, and by age to <38 or ≧38 years. <35 (but not ≧35) repeats demonstrated significantly lower AMH at ages ≧38 than <38 years (p<0.05). >38 years, AMH was not affected by repeats. <38 years, with <35 repeats (though not with ≧35), required significantly less gonadotropins than ≧38 years (p<0.05). <38 years (though not ≧38), those with <35 repeats produced significantly more oocytes than women with ≧35 repeats (p=0.006). <38 years, retrieved oocytes were inversely related to repeats, adjusted for gonadotropin dosage (p=0.03). This supports FMR1 testing as useful in fertility practice and suggests why response rates to increasing stimulation with gonadotropins may vary.

In sum, the number of triple CGG repeats on the FMR1 gene, represent a useful test in determining risk towards premature ovarian senescence and infertility.

### A Study of FMR1 Gene and Infertility

After the studies described above, we continued our testing and analyzing of CGG repeat counts on the FMR1 gene.

As previously discussed herein, excessive expansion sizes of triple CGG repeats on the FMR1 (fragile X) gene predispose females towards premature ovarian senescence. This observation was first made when the risk of premature ovarian failure (POF), the most severe manifestation of premature ovarian senescence, was noted increased in carriers of premutations (55-200 repeats). Milder forms of premature ovarian senescence, often characterized by laboratory evidence of prematurely decreased ovarian reserve [i.e., elevated baseline follicle stimulating hormones (FSH)], however, have more recently also been reported at premutation, so-called intermediate (or “gray zone”) ranges (45-55 repeats) and even with high normal triple repeat sizes.

These observations raise the obvious question whether the number of triple CGG repeats on the FMR1 gene may not linearly be reflective of ovarian reserve and, therefore, denote risk towards female infertility? Such lower expansion sizes, at normal and low to middle ranges, are, therefore, very relevant topics of possible investigations because, assuming such a statistical association, lower expansion ranges may reflect relatively milder degrees of ovarian dysfunction, diagnostically more challenging than the clinically rather obvious diagnosis of POF. Like an initial report by Bretherick et al, a pilot study at our own center suggested that the risk for premature ovarian senescence (at least in its mildest forms) may already start below the so-called intermediate range, at generally considered normal expansion numbers of up to 45 repeats.

No attempts were ever made to directly correlate individual triple CGG numbers (rather than ranges) with gradual ovarian function parameters (rather than defined ovarian function ranges) in either normal populations or infertile women. Assuming a functional correlation between triple CGG repeat numbers and ovarian function, such a correlation should, at least in selected ranges, be detectable.

We in an earlier study were, indeed, able to confirm such a correlation. This study, however, because of small patient numbers and some outliers at very high repeats (full mutations), lacked a truly satisfactory degree of statistical power and was, therefore, designated as a pilot study. The here presented data, omitting these outliers, and presenting a much larger patient volume, are, therefore, much more satisfactory.

Confirming the correlation between CGG repeat numbers and ovarian reserve has significant potential clinical implications: It suggests a role of the FMR1 gene product, the so-called fragile X mental retardation protein, FMRP, on ovarian function, following other gonadal effects reported. Even nanomolar concentrations FMR1 apparently strongly inhibit translation of various mRNAs in microinjected Xenopus laevis oocytes, thus down regulating translation. Bächner et al, demonstrating FMR1 expression in fetal (though not adult) mice ovaries, suggested a special function during germ cell proliferation. Gene expression in the human fetal ovary was reported by Rifė and associates.

Confirmation for such correlation also leads to the ability to utilize the FMR1 gene analysis in infertility diagnostics. Quantitating risk towards premature ovarian senescence, based on triple CGG repeat numbers, improves the diagnosis of female infertility, allowing better reproductive planning and, when needed, facilitating attempts at fertility preservation.

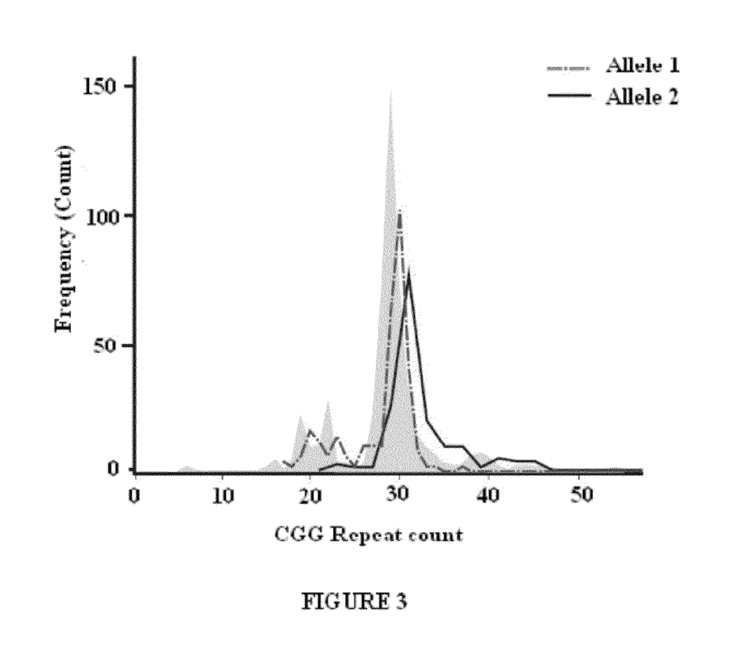

In this study, our objective was to determine whether triple CGG numbers on the FMR1 gene correlate with ovarian reserve and, thus, facilitate infertility diagnosis. This study also adds to the understanding of reported triple CGG repeats in the general population and amongst women with infertility: Though up to 45 CGG repeats are considered normal, Fu et al reported that most humans demonstrate between 29 and 30 repeats (the shaded region of FIG. 3). Their distribution curve, therefore, demonstrates a very large peak at these triple CGG numbers, with much smaller patient populations to the right (higher counts) and to the left (lower counts). From a statistical point it is, therefore, tempting to consider individuals with equal or less than 28, and 31 or more, triple repeats, outside of an expected “standard” range. Assuming, once more, that excessive triple CGG numbers reflect risk towards premature decline in ovarian reserve, distributions to the right of above noted peak may, therefore, represent at least a portion of those approximately 10 percent of all females who have been suggested to suffer from premature ovarian senescence, a prevalence even much higher amongst infertile women.

Assuming all of this to be correct, then infertile women may be expected to demonstrate a CGG triple repeat pattern which in comparison to the general population is moved towards the right (higher repeat numbers). This study, therefore, also investigated the distribution pattern of CGG repeats on the FMR1 gene in infertile women and compared it statistically with the distribution pattern, previously reported by Fu et al for the general population. See FIG. 3 and at least Example 3 below.

To avoid statistical confusion from excessively high CGG count outliers, we in this study concentrated only on counts between 35 and 50 CGG repeats, the upper two thirds of what currently is considered normal, and the lower half of the so-called intermediate (“gray”) zone. Lower triple CGG ranges have also been reported to demonstrate more linear correlations with ovarian function than higher ranges. We are also utilizing in this manuscript the previously coined acronym premature ovarian aging (POA) for milder forms of premature ovarian senescence (in differentiation from POF) which differs in some aspects from normal physiologically aging. For example, POA patients do not demonstrate increased embryo aneuploidy, as is associated with physiologic female aging.

Our results showed that 158 infertile women demonstrated a shift towards higher counts, as compared to the general populations. Counts inversely correlated with AMH (R²=0.21; p<0.001), though not FSH, primarily attributable to women under about 38 years old (R²=0.26; p<0.001). Up to about 40 years, <35 triple repeats demonstrated higher AMH levels than 35 to 50 repeats [F (1.87)=5.3, p=0.025]. Between about 35-50 repeats inversely correlated to AMH (R²=0.41; p<0.013).

In sum, premature ovarian senescence and infertility are statistically associated with increasing triple CGG numbers on the FMR1 gene, representing a useful test in diagnosing risk towards diminished ovarian reserve and female infertility.

### A Study of FMR1 Gene and Race/Ethnicity

A variety of observations point towards racial/ethnic genetic variations, and therefore, there are likely racial/ethnic fertility parameter variations. Many suggest distinct differences in ovarian function. For example, the prevalence of premature ovarian failure (POF) varies with ethnicity (Luborsky et al., 2003). We have reported that young Chinese egg donors exhibit significantly more prematurely diminished ovarian reserve than Caucasian donors (Gleicher et al., 2007) and suggested that this observation may be one explanation for the reported poorer pregnancy rates of Chinese women after in vitro fertilization (IVF) (Purcell et al., 2007). Complementing the picture, Greenseid et al reported that Caucasian women with diminished ovarian reserve conceive with higher probability than women of other races (Greenseid et al., 2004) and Montgomery et al suggested that Caucasian women also produce more follicles (Montgomery et al., 2006). Research on Hutterites suggested a genetic background to reproductive fitness (Pluzhnikov et al., 2007).

The fact that the FMR1 gene may be relevant to ovarian function and female fertility has been known for some time (Wittenberger et al., 2007). In animal models it has been associated with germ cell proliferation in males and females (Bächner et al., 1993). In humans, gene expression has been reported in the fetal ovary (Rifė et al., 2004). Women demonstrate increased risk for POF with excessively high triple CGG repeat numbers, especially at premutation range (55-200 repeats) (Lubosrsky et al., 2003; Wittenberger et al., 2007), but also at lower counts (Murray et al., 2000; Bodega et al., 2006).

Milder forms of premature ovarian senescence have recently also been reported, associated with increasing CGG counts (Murray et al., 2000; Hundscheid et al., 2001; Welt et al., 2004; Allen et al., 2007) and we reported that risk for premature ovarian senescence appears to increase in both directions from the large distribution peak between 29 and 30 CGG triple repeats, which characterizes a large majority of humans (Gleicher et al., 2009a; Gleicher et al., 2009b; Gleicher et al., 2009c).

Whether excessive triple CGG counts are equally distributed amongst different races/ethnicities, and whether they denote the same risk towards premature ovarian senescence in different racial/ethnic groups is, however, still unknown. A recent document, issued by the Fragile X Premature Ovarian Insufficiency Working Group of the National Institutes of Health (NIH), identified this question as a desirable target for further investigations [Fragile X-Primary Ovarian Insufficiency (FX-POI) Working Group; 2008]. This study was, therefore, designed to investigate this question and whether different ethnicities/races, in parallel, may not also demonstrate variations in selected ovarian function parameters.

We investigated in a cross-sectional cohort study of 385 females (770 FMR1 alleles) the number of CGG repeats on the FMR1 (fragile X) gene, and whether there were differences between races/ethnicities. The study population involved 344 infertility patients and 41 oocyte donors. See at least Example 4 below.

For purposes of this study, a normal CGG count range was not defined by standard or traditionally utilized definitions of potential neuro-psychiatric risks (common, intermediate, premutation, full mutation ranges), but by box and whisker plot, representing a standard method of assessing normal ranges in general populations. Using this method, the range of about 20-about 35 repeats were defined, preferably about 24-about 34 repeats were defined, most preferably about 26-about 34 repeats (median 30 repeats) were defined as normal range for the whole study population, and individually reconfirmed for all investigated races/ethnicities. In sum, 26 CGG repeats—34 CGG repeats represents a normal range with regard to ovarian function/reserve. The distribution of abnormal outliers in CGG counts from the most preferable normal range was then compared between women of Caucasian, African and Asian descent.

African and Asian women demonstrated a higher prevalence of two normal count alleles (65%) than Caucasians (54.3%; p=0.03). Caucasians demonstrated the highest rate of allele abnormalities (43.3%) and were the only race/ethnicity also demonstrating abnormalities in both alleles of FMR1. Asian women demonstrated significantly fewer low outlier counts than Caucasians (p=0.0020) and Africans (p=0.03).

This study, thus, suggests significant racial/ethnic differences in triple CGC counts on the FMR1 gene between races/ethnicities. Since CGG counts on FMR1 are associated with ovarian reserve, these findings reflect potential differences between races/ethnicities in ovarian function and female fertility reported in the literature.

### A Study of FMR1 Gene and Regulation of Ovarian Reserve

Recently, testing of the FMR1 (fragile X) gene has been recommended in women with prematurely diminished ovarian reserve, as they demonstrate abnormally elevated CGG counts. Human investigations have been limited, but gene expression has been reported in fetal ovaries.

These observations raise the question whether the FMR1 gene, in addition to its neuro/psychiatric risks, does not also influence ovarian reserve. A first association between CGG counts on the FMR1 gene and ovarian reserve was made in so-called premature ovarian failure (POF) when excessive triple CGG repeats in premutation range were associated with POF. Later, also lower CGG counts were found associated with premature ovarian senescence.

We speculated about a possibly more general statistical correlation between CGG counts and ovarian reserve when intermediate (45-54) and high typical (i.e., normal) (<45) repeat ranges were associated with milder forms of premature ovarian senescence, and then demonstrated such correlations in a number of studies, independent of an apparently autoimmune-driven risk towards premature ovarian senescence.

The distribution pattern of CGG repeats in general populations is characterized by a domineering peak between about 29 and about 30 CGG repeats. After determining that infertile female women demonstrate a very similar pattern, we considered the hypothesis that this distribution peak may represent normal in reference to a potentially new function of the gene, which relates to ovarian reserve. CGG repeats to the right and left of this peak then would represent abnormal outliers, potentially reflective of abnormal ovarian reserve.

This hypothesis proved correct, when increasing and decreasing counts were shown to almost equally predispose towards premature ovarian senescence. As a next step, using box and whisker plots, we then determined the range of about 20-about 35 repeats as normal range for CGG counts in reference to ovarian reserve and confirmed the consistency of this normal range in women of varying ethnicities, preferably about 24-about 34 repeats, and most preferably about 26-about 34 repeats (median 30). This range, of course, also correlated well with the previously noted distribution peak in the general population between about 29 and about 30 repeats, originally reported by Fu et al but has to be differentiated from currently considered so-called typical (normal) CGG counts in assessing neuro/psychiatric risks. In sum, 26 CGG repeats to 30 CGG repeats represents a normal range with regard to ovarian function/reserve, as opposed to 40 CGG repeats to 45 CGG repeats representing normal with regard to neuro-psychiatric risks.

Fu's and our data previously were, however, all based on analyses of single FMR1 alleles and, therefore, may not comment on either heterozygosity (one allele in abnormal range) or homozygosity (both alleles abnormal). This study, therefore, assesses for the first time the impact of heterozygosity and homozygosity in triple CGG repeats on ovarian reserve.

Our objective was to explore whether in two-allele analysis of two normal (normal), one abnormal (heterozygous) or two abnormal (homozygous) alleles if the FMR1 (fragile X) gene reflects risk towards diminished ovarian reserve (DOR) better than single allele analysis. See at least Example 5 below.

Our patients included 34 young oocyte donors, and 305 female infertility patients. To perform our analysis, we had to determine the number of triple CGG repeats on each allele of the FMR1 gene and designate whether the number was normal or heterozygous or homozygous when compared to the range. In addition to studying the alleles, we also used the patients Anti-Müllerian hormone (AMH) levels, as reflection of ovarian reserve, in our analysis.

Our results confirm by box and whisker plot, for the whole study population, a normal range of about 20-about 35 CGG repeats, preferably about 24-about 34 CGG repeats, and most preferably about 26-about 34 CGG repeats (median 30 CGG repeats). Women with normal alleles exhibit at young ages significantly higher AMH levels than either heterozygous or homozygous abnormal females (p=0.009). Further, by about age 35 heterozygous women, however, start exceeding AMH levels of normal women, while homozygous women cross normal females shortly before about age 50 years.

In sum, the data further supports control of ovarian reserve by the FMR1 gene, and, in reference to ovarian function, a normal CGG count, of about 26-about 34 (median 30). The gene appears to define life-long ovarian reserve patterns, with abnormal counts reducing ovarian reserve at younger, but improving it at advanced ages. The FMR1 gene, thus, may preserve fertility into older age at the expense of reducing fertility at younger ages.

### Continued Studies of FMR1 Gene

Continuing the studies described above, we further have tested and analyzed CGG repeat counts on the FMR1 gene.

First, we focused on whether various ovarian reserve parameters improve donor selection, and we concluded that utilization of new markers of ovarian reserve, such as AMH and CGG repeats one FMR1, will likely improve donor selection, thus reducing risks towards disappointing oocyte yields and hyperstimulation. See at least Examples 6 and 6A below.

Second, we focused on the fact that normal and heterozygous (het) or homozygous (hom) abnormal counts on the FMR1 gene reflect distinct ovarian aging curves. Het abnormal can, however, be normal/low or normal/high, which may affect ovarian reserve (OR) differently. We then analyzed whether OR, reflected by anti-Müllerian hormone (AMH) and oocytes yield (phenotype), differs depending on FMR1 genotype, in 4 groups (norm, het-1, het-2, hom), stratified for age <35 and ≧35 years. We concluded that refinement in CGG count analysis of het abnormal women demonstrates distinct differences in ovarian aging patterns between het-1 and het-2 genotypes. Based on initially very high AMH in het-1 and observed habitués, the het-1 genotype appears to represent (normal weight) women with non-typical PCOS, who at young age unusually rapidly loose OR. See at least Example 7 below.

Third, we focused on the effects of CGG repeats when both alleles are in normal (norm) range or either 1 allele [heterozygous (het)] is abnormal or 2 alleles [homozygous (hom)] are abnormal. We concluded that ovarian aging differs based on norm, het or hom CGG counts, confirming an association between CGG repeats and ovarian reserve, and suggesting an FMR1 effect on follicular recruitment in favor of follicular preservation and fertility extensions into older age. Such functions potentially contribute to species maintenance, which may explain why the FMR1 gene is highly preserved despite obvious, and at often severe medical consequences primarily in males. See at least Example 8 below.

### Additional Studies of FMR1 Gene

Associations between number of triple CGG nucleotide repeats on the fragile X mental retardation 1 (FMR1) gene and risk towards premature ovarian senescence have been reported, leading in milder cases to so-called premature ovarian aging (POA), also called occult primary ovarian insufficiency (OPOI), and at end stage to premature ovarian failure (POF), also called primary ovarian insufficiency (POI). We recently reported evidence that different FMR1 genotypes vary in rate of follicle recruitment and, therefore, at least partially, affect functional ovarian reserve, as assessed by anti-Müllerian hormone (AMH) (as discussed herein, see at least Example 5).

A normal triple nucleotide (CGG) count range of 26 to 34 repeats (median 30), in respect to ovarian function, allows definition of distinct FMR1 genotypes, depending on whether both (normal), only one (heterozygous) or neither (homozygous) allele is in normal range. In a small pilot study a heterozygous-normal/low (het-norm/low) sub-genotype appeared associated with a lean polycystic ovary (PCO)-like phenotype with rapidly depleting ovarian reserve.

A PCO-like phenotype is integral to all definitions of the polycystic ovary syndrome (PCOS). The phenotype, however, solely denotes excessive follicle activity (reflected in high AMH) and, consequently, hyperactivity of ovarian function. PCO and PCOS, therefore, have distinctively different connotations.

Some have speculated about a possible autoimmune etiology for selected forms of PCOS. Others implied such an association when histological demonstrating autoimmune oophoritis with polycystic aspects, accompanied by anti-ovarian antibodies.

At the other end of ovarian function, autoimmunity has been for decades implicated in POF/POI. A very specific phenotype, characterized by a preserved pool of functional follicles, recently has been associated with steroidogenic cell autoimmunity. Hypo-activity is, thus, well documented in association with autoimmunity, while hyperactivity of ovaries is not.

Considering the substantial evidence in support of autoimmune-associated suppression of ovarian function, we have speculated about autoimmune-induced ovarian stimulation in PCOS patients, which would mimic the independent duality of, for example, thyroid autoimmunity and function, both etiologically linked, interacting, yet relatively independent of each other.

At first, our initial efforts through pilot studies at our center to demonstrate evidence for autoimmune activity in association with PCOS presented challenges. We attributed the difficulties to phenotypical and etiologic variabilities of PCOS, as defined by current Rotterdam criteria. However, identifying above noted PCO-like phenotype with close association to the het-norm/low FMR1 sub-genotype offered a unique opportunity to study a clinically homogenous PCO-like patient population.

We now confirm close associations between FMR1 genotypes and ovarian function. For the first time, we associate FMR1 genotypes in infertile women with risk towards autoimmunity and with pregnancy chances in association with in vitro fertilization (IVF). See at least Example 9 below.

### Remarks on the FMR1 Gene Studies

Although evidence from animal studies suggests that ovarian reserve is genetically controlled, it was undetermined how this genetic control works in humans. We have studied the effect of the FMR1 gene on ovarian reserve and for the first time demonstrate that the FMR1 gene plays a crucial role in ovarian aging. Probably most importantly, we defined new FMR1 genotypes and sub-genotypes, associated with specific ovarian aging patterns. In other words, based on a young girl's FMR1 gene pattern, one can predict how she, likely, will age her ovaries.

Almost all of our studies have been done in infertile women. While one can extrapolate, things may, nevertheless, be different in normally fertile women, especially while they are still young and unaffected by the physiological process of ovarian aging.

We, therefore, in one study, decided to investigate FMR1 genotypes and sub-genotypes in normal young egg donors, who had successfully donated. And low and behold, even at these very young ages there were already very significant differences in ovarian reserve between FMR1 genotypes and sub-genotypes.

These observations are of great potential clinical and scientific significance for a number of reasons: these findings will find their most practical and immediate application in egg donor selection. We now have another, highly accurate tool in selecting only the best possible donors for our patients.

The data also reemphasizes the importance of the FMR1 gene for female infertility. Women start their reproductive lives with different levels of ovarian reserve, and deplete their ovarian reserves at different speeds. How much ovarian reserve women start with and how quickly they depletes, of course, is the perfect definition of—“ovarian aging.” The FMR1 gene, therefore, can be viewed as the ovarian aging gene.

One can foresee that this new knowledge about the genetic regulation of ovarian aging via the FMR1 gene will lead to better diagnostic tools and more successful therapeutic interventions in female infertility.

### Additional Remarks on the FMR1 Gene as Regulator of Ovarian Recruitment and Ovarian Reserve

Until now, nobody recognized that the permutation range of the CGG repeats may point toward a possible ovarian control function of the FMR1 gene may point toward a possible ovarian control function of the gene.

Assessing a woman's CGG repeat numbers on the FMR1 gene may establish that a woman faces increased risk toward premature ovarian aging. Once this assessment is done, then a woman may be counseled and follow-up with regular ovarian reserve tests, such as anti-Müllerian hormone (AMH) levels. If such testing then reveals that a woman's ovarian reserve is prematurely diminishing, then she has adequate time to appropriately modify her reproductive planning and/or take steps to preserve fertility potential through assisted reproduction methods such as egg or embryo cryopreservation.

Ovarian function in all races/ethnicities appears defined by a normal range of 26 to 34 CGG repeats (mean 30), including the reported distribution peak of 29 to 30 repeats in humans and maximal gene translation, reported at 30 repeats.

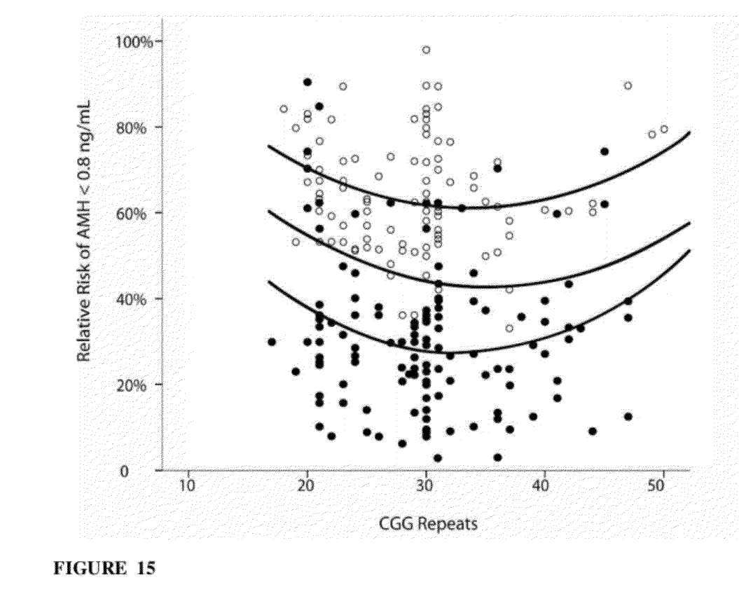

Additionally, high CGG numbers (above 34) and low CGG numbers (below 29), both reflect risk toward POA/OPOI. FIG. 15 is a logistic regression of DOR (low AMH) and CGG counts, adjusted for age. The figure represents logistic regressions, demonstrating predictive relative risk of AMH <0.8 ng/mL over CGG counts, stratified for age—the upper curve is ≧38 years of age; the lower curve is <38 years of age; the middle curve is all ages. As shown in FIG. 15, every 5 CGG repeats below 30 increases the relative risk for low AMH by about 60%; Every 5 CGG repeats above 30 increases the relative risk for low AMH by about 40%. Open circles are women under age 38 and closed circles represent women above age 38 years. See FIG. 15.

Genotypes, defined by 2 normal count alleles (normal) demonstrate different OR aging patterns from women with 1 (heterozygous) or both alleles outside of range (homozygous). Heterozygous and homozygous genotypes recruit fewer follicles at younger ages, thus preserving OR into advanced age.

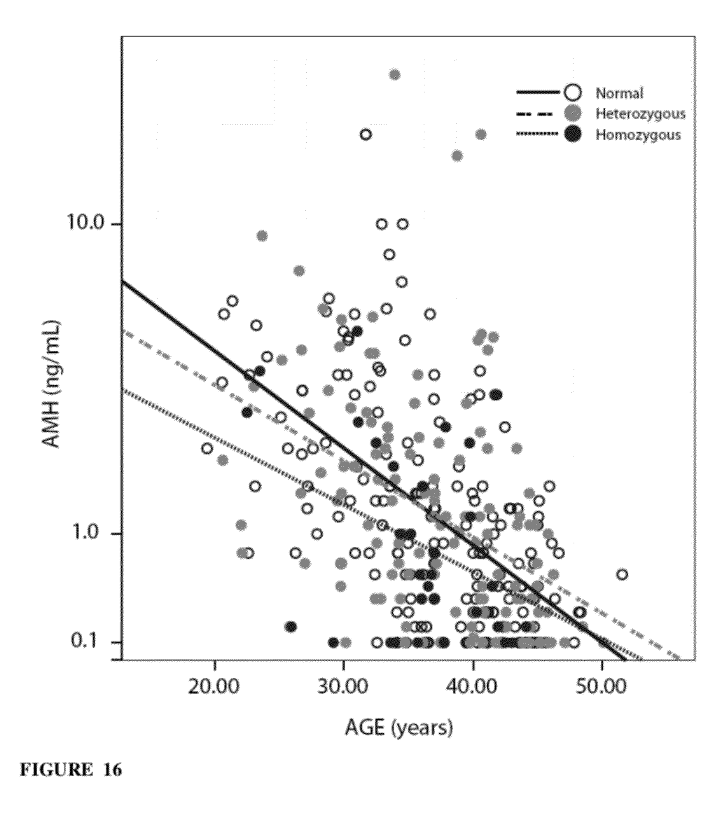

In evaluating the OR controlling function of the FMR1 gene, at young ages: OR was the best in norm women, followed by het patients, with hom patients demonstrating the lowest reserve. OR patterns, however, changed with advancing age: women with norm genotype rapidly declined, while het and hom patients “aged” ovaries at a slower pace. Consequently, by about age 33 to about 34 years, AMH in women with norm genotype dropped below that of het genotypes and even below het genotypes in the late 40s. See at least FIG. 16. FIG. 16 shows a linear regression of AMH levels, depending on genotypes norm, het, and hom. The regression of norm patients crosses regression lines of het at approximately age 34 and of hom at approximately age 47 years.

Norm women deplete their OR much quicker than either het or hom patients. Thus, 2 alleles with normal CGG counts appear associated with active utilization of follicles/oocytes at young ages, and therefore, relatively rapid decline in remaining follicles, while het and hom genotypes demonstrate poorer recruitment at young ages and, therefore, comparably lower functional OR at such young ages, as assessed by AMH. Because of slower recruitment of primordial follicles at younger ages, these women, however, maintain a larger follicle pool into older age and, at more advanced ages, demonstrate better OR than norm women.

These observations suggest a direct FMR1 effect on follicular recruitment and OR and, therefore, on women's fecundity.

FMR1 Genotypes are Associated with Cancer Risks

Ovarian genotypes and sub-genotypes of the FMR1 gene in 99 BRCA1/2 mutation-positive Austrian breast cancer patients were investigated. There were significant differences in distribution as compared to 410 U.S. control females. BRCA1/2 carriers almost exclusively demonstrated only FMR1 genotypes and sub-genotypes with at least one low (CGG n<26) allele. This suggests embryo-lethal effects of BRCA1/2 in humans, unless embryos are rescued by low FMR1 genotypes or sub-genotypes. Cross tabulation between the comparison group and BRCA1/2-positive cancer patients confirmed significant group membership, related to FMR1 distribution (P<0.0001). If low genotypes/sub-genotypes of FMR1 rescue human embryos from BRCA1/2-associated lethality, they have to reflect increased cancer risks associated with BRCA1/2 mutations. Occurring in approximately 25 percent of women, low FMR1 genotypes and sub-genotypes, therefore, expand risk for BRCA1/2-associated cancers to approximately one quarter of the female population, while reducing risk in the remaining three-quarters, potentially revolutionizing cancer screening for breast, ovarian and other malignancies in women. In short, BRCA1/2 are embryo lethal human gene mutations, which are rescued by low count (CGG n<26) FMR1 mutations. As such, women with CGG n<26 may suggest a new target of females that may be screened for female cancer, such as breast and/or ovarian cancer. See at least Example 10 below.

### The Impact of Androgens On Pregnancy Potential In Women With Diminished Ovarian Reserve

For decades, androgens have been considered detrimental to follicle maturation. Animal studies now suggest that they are essential for normal folliculogenesis. Recent in vitro fertilization (IVF) data in humans, especially in women with diminished ovarian reserve (DOR), supports that androgens positively impact folliculogenesis. There is an association between recently reported ovarian genotypes of the FMR1-gene and ovarian aging patterns. We, therefore, attempted to elucidate the endocrinology of androgen supplementation, and its effects on pregnancy potential in women with diminished ovarian reserve, and whether these effects change in association with FMR1-genotypes.

We longitudinally assessed androgen metabolisms in 91 women with premature DOR, following pre-supplementation with micronized dehydroepiandrosterone (DHEA) prior to IVF. IVF-outcomes were assessed based on androgen levels and ovarian FMR1-genotypes.

The mean age was 39.8±4.4 years; and the clinical pregnancy rate was 25.3%. Total androgen concentrations were not associated with pregnancy; however, in women with heterozygous and homozygous FMR1-genotypes, but not with normal genotype, free testosterone significantly affected clinical pregnancy potential (β=1.101, SE±0.508, p=0.03), increasing pregnancy potential by a factor of 3. At IVF cycle start, interactions of DHEA with total and free testosterone also significantly affected pregnancy rates (β=−0.058, SE±0.023, p=0.01 and β=−0.496, SE±0.197, p=0.012), decreasing for every unit increase by a factor of 1.06 and 1.64, respectively.

In sum, androgen interactions significantly influence IVF-pregnancy rates in women with prematurely DOR, with impact of total androgens on cycle outcomes varying according to FMR1-genotypes. These observations suggest that the effectiveness of androgen supplementation in women with DOR varies based on FMR1-genotypes, and defines androgen deficiency as a characteristic of DOR. See at least Example 11 below.

### Various Aspects of the Method and Apparatus

A method of predicting a degree of risk of early ovarian aging of a young female is disclosed. The method may include analyzing the female's FMR1 gene, wherein the FMR1 gene has a first allele and a second allele, determining the number of triple CGG repeats on each of the first and second alleles, defining a normal range of triple CGG repeats, and comparing the number of triple CGG repeats on each of the first and second alleles to the normal range. If the triple CGG repeat numbers for both of the first and second alleles are within the normal range, then the female is at minimal risk for early ovarian aging. If the triple CGG repeat number for one of the first and second alleles is outside of the normal range and the other one of the first and second alleles is within the normal range, then the first and second alleles are heterozygous and the female is at increased risk for early ovarian aging. If the triple CGG repeat numbers for both of the first and second alleles are outside of the normal range, then the first and second alleles are homozygous and the female is also at an increased risk for early ovarian aging. The normal range may be between 26 and 34 triple CGG repeats with regard to ovarian function/reserve. Further, a young female may be a female that is under the age of 35.

The method may also include confirming ovarian aging with a secondary test. The secondary test may analyze at least one of FSH and anti-Müllerian hormone levels. Also, the secondary test may analyze the female's oocyte yield. The method may further include evaluating autoimmune status by testing at least one antiphospholipid antibody panel, antinuclear antibody panel, total immunoglobulin levels, thyroid antibodies, antiovarian, and antiadrenal antibodies.

The first and second alleles may be heterozygous such that the first allele may be abnormal-high and the second allele may be normal. Alternatively, the first and second alleles may be heterozygous such that the first allele may be abnormal-low and the second allele may be normal.

If the triple CGG repeat numbers for both of the first and second alleles are outside of the normal range, then the first and second alleles are homozygous and the female is also at an increased risk for early ovarian aging.

Also, the first and second alleles may be homozygous such that the first and second alleles may be homozygous-low. Alternatively, the first and second alleles may be homozygous such that the first and second alleles are homozygous-high.

Further, a method of determining current ovarian function in a female is disclosed. The method may include analyzing the female's FMR1 gene, wherein the FMR1 gene has a first allele and a second allele, determining the number of triple CGG repeats on each of the first and second alleles, defining a normal range of 26 triple CGG repeats, comparing the number of triple CGG repeats on each of the first and second alleles to the normal range. If the triple CGG repeat number for at least one of the first and second alleles is less than the normal range, then the first and second alleles are heterozygous and the female is at an increased risk of having diminished ovarian function.

Additionally, a method of determining current ovarian function in a female is disclosed. The method may include analyzing the female's FMR1 gene, wherein the FMR1 gene has a first allele and a second allele, determining the number of triple CGG repeats on each of the first and second alleles, defining a normal range of 34 triple CGG repeats, and comparing the number of triple CGG repeats on each of the first and second alleles to the normal range. If the triple CGG repeat number for at least one of the first and second alleles is greater than the normal range, then the first and second alleles are heterozygous and the female is at an increased risk of having diminished ovarian function.

Moreover, a method for determining etiology of infertility in a female is disclosed. The method may include testing a DNA sample from a female for triple CGG repeat number in first and second alleles of the FMR1 gene and correlating a triple CGG repeat number outside of a normal range on the first and/or second alleles of the FMR1 gene with a genetic etiology of the infertility, wherein the female has been diagnosed as infertile.

The female may have been unsuccessful in conceiving after at least 6 months of attempting to become pregnant. Further, the female may have been unsuccessful in conceiving after at least 12 months of attempting to become pregnant.

The method for determining etiology of infertility in a female may also include determining presence of autoimmunity in the female. Determining presence of autoimmunity may include determining presence of symptoms of autoimmune disorders in the female. Further, determining presence of autoimmunity may include testing the female for autoimmune antibodies.

Also, a method of determining increased risk of premature ovarian aging in a female is disclosed. The method may include testing a DNA sample from a female for triple CGG repeat number in first and second alleles of the FMR1 gene, and correlating a triple CGG repeat number outside of a normal range on the first and/or second alleles of the FMR1 gene with increased risk of premature ovarian aging, wherein the female is considering delaying reproduction.

The female may be at most 35 years old. Alternatively, the female may be at most 30 years old. In a further alternative, the female is at most 25 years old. In an additional alternative, the female may be between 18-25 years old.

The method of determining increased risk of premature ovarian aging in a female may further include determining presence of autoimmunity in the female. The method may also include determining presence of autoimmunity comprises determining presence of symptoms of autoimmune disorders in the female. Further, the method may include determining presence of autoimmunity comprises testing the female for autoimmune antibodies.

Also, the method of determining increased risk of premature ovarian aging may include reporting results of the testing to the female. The reporting may include sending the results of the testing to the female. The reporting further may include making the results available to the female on a website. Further, the method may include providing to the female a kit for collecting the DNA sample. The kit may include a container for holding the DNA sample, and a mailer for sending the DNA sample to a testing laboratory. Additionally, the method may include testing AMH levels of the female, wherein the female is at least 25 years old.

Moreover, a kit for determining increased risk of premature ovarian aging is disclosed. The kit may include a tissue sampler, a mailer preaddressed for a laboratory, and instructions including directing collection of tissue from a female considering delaying reproduction. The kit also may include a questionnaire containing questions regarding symptoms of autoimmune disorders. The tissue sampler may be a swab for collecting tissue from within the female's mouth. Further, the tissue sampler may be a blood sampler. The instructions may include instruction for collecting test results from a website.

Additionally, a method of using a kit is disclosed. The kit may include a tissue sampler, a mailer preaddressed for a laboratory, and instructions comprising directing collection of tissue from a female considering delaying reproduction. The method may include collecting the tissue following the instructions, mailing the tissue to a laboratory for evaluation, receiving results from the laboratory, wherein the results include the number of CGG repeats on each allele of the female, and evaluating the results. Evaluating the results may include comparing the number of CGG repeats on each allele to a normal range, and determining whether the number of CGG repeats are within the normal range, and if either number of CGG repeats is outside of the normal range, then the female is at an increased risk for early ovarian aging, autoimmunity and/or cancer.

Even further, a method of predicting a degree of risk of autoimmunity in a human female is disclosed. The method may include analyzing the female's FMR1 gene, wherein the FMR1 gene has a first allele and a second allele, determining the number of triple CGG repeats on each of the first and second alleles; defining a normal range of triple CGG repeats; and comparing the number of triple CGG repeats on each of the first and second alleles to the normal range. If the triple CGG repeat number for one of the first and second alleles is in the normal range and the triple CGG repeat number for the other one of the first and second alleles is less than the lower boundary of the normal range, then the female is at increased risk of autoimmunity.

Additionally, a method of predicting pregnancy chances for a human female is disclosed. The method may include analyzing the female's FMR1 gene, wherein the FMR1 gene has a first allele and a second allele; determining the number of triple CGG repeats on each of the first and second alleles; defining a normal range of triple CGG repeats; and comparing the number of triple CGG repeats on each of the first and second alleles to the normal range. If the triple CGG repeat number for one of the first and second alleles is in the normal range and the triple CGG repeat number for the other one of the first and second alleles is less than the lower boundary of the normal range, then the female has decreased chances of pregnancy.

Further, a method of predicting a degree of risk of autoimmunity in a human female is disclosed. The method may include analyzing the female's FMR1 gene, wherein the FMR1 gene has a first allele and a second allele, determining the number of triple CGG repeats on each of the first and second alleles; defining a normal range of triple CGG repeats; and comparing the number of triple CGG repeats on each of the first and second alleles to the normal range. If the triple CGG repeat number for one of the first and second alleles is in the normal range and the triple CGG repeat number for the other one of the first and second alleles is greater than the upper boundary of the normal range, then the female is at a decreased risk of autoimmunity. Similarly, if the triple CGG repeat number for one of the first and second alleles is in the normal range and the triple CGG repeat number for the other one of the first and second alleles is greater than the upper boundary of the normal range, then the female is protected from autoimmunity.

Additionally, a method of screening a human for risk of malignancies is disclosed. The method may include isolating the human's FMR1 gene, wherein the FMR1 gene has a first allele and a second allele, measuring the number of triple CGG repeats on each of the first and second alleles, wherein the measuring step is conducted through use of an assay, and identifying the human as at risk for cancer when the triple CGG repeat number for at least one of the first and second alleles is less than 26. The method also may include identifying the human as at risk when the triple CGG repeat number for at least one of the first and second alleles is between 26-34. The method may include identifying the human as at risk when the triple CGG repeat number for each of the first and second alleles is less than 26. The method further may include identifying the human as at risk when the triple CGG repeat number for at least one of the first and second alleles is greater than 34. Also, the method may include identifying the human as at risk for at least one of breast cancer and ovarian cancer. The human may be female. The assay may be at least one of Southern blotting and polymerase chain reaction. Additionally, the method may include conducting a secondary test by isolating the human's BRC1 gene and the BRC2 gene, analyzing each of the BRC1 and BRC2 genes for mutations by performing at least one of denaturation high performance liquid chromatography and chain-terminating inhibitors. Also, the method may include confirming the presence of cancer by conducting a secondary test and analyzing the results of the secondary test. The method may include administering to the human at least one a gene blocker a therapeutic drug to interfere with the cancer.

Moreover, a method of screening a human female for the clinical effectiveness of an androgen in a human female is disclosed. The method may include measuring the female's testosterone levels, administering an androgen to the female, isolating the female's FMR1 gene, wherein the FMR1 gene has a first allele and a second allele, measuring the number of triple CGG repeats on each of the first and second alleles, wherein the measuring step is conducted through use of an assay, measuring the female's testosterone levels again, identifying the androgen as clinically effective such that the female has an increased pregnancy potential when the female's testosterone levels were higher when measured again and when the triple CGG repeat number for at least one of the first and second alleles is less than 26 or greater than 34.

Furthermore, a method of screening a human for increased embryo quality is disclosed. The method may include isolating at least one of the human's BRC1 and BRC2 genes, analyzing each of the BRC1 and BRC2 genes for mutations, isolating the human's FMR1 gene, wherein the FMR1 gene has a first allele and a second allele, when a mutation of at least one of the BRC1 and BRC2 gene is present, measuring the number of triple CGG repeats on each of the first and second alleles, wherein the measuring step is conducted through use of an assay, and identifying increased embryo quality and increased embryo survival, when the triple CGG repeat number for at least one of the first and second alleles is less than 26. The androgen may be DHEA.

### EXAMPLES

The following detailed examples are to be construed as merely illustrative and not limitative of the disclosure in any way.

### Example 1

The objective of this study was to determine to what degree milder forms of premature ovarian aging are also associated with two etiologies: the presence of excessive triple CGG expansions on the FMR1 (fragile X) gene (genetic etiology) and a variety autoimmune conditions (autoimmune etiology).

### Materials and Methods of Example 1

We investigated 119 consecutive infertility patients with clinical evidence of diminished ovarian reserve (DOR). A diagnosis of DOR was reached if age-specific baseline follicle stimulating hormone (FSH) levels were abnormally elevated and/or if patients demonstrated ovarian resistance to stimulation with gonadotropins. The determination of elevated baseline FSH levels is at our center made age-stratified and specific to the center's patient population, with abnormal, at each age, representing levels that exceed the 95% confidence interval of FSH levels for this particular age group.

Study patients were then investigated for the number of triple CGG expansions of both alleles of their FMR1 gene, using commercially available assays. The allele with lower count CGG repeats was designated allele-1 and the one with a higher number as allele-2.

Because infertility patients demonstrate an increased prevalence of autoimmune abnormalities, often asymptomatic, our fertility center routinely performs a so-called immune screen on new patients entering treatment. It consists of antinuclear antibody, a limited antiphospholipid antibody (anticardiolipin, antiphosphatidylserine and β2-glycoprotein antibodies in IgG, IgM and IgA isotypes) and thyroid antibody panels (antithyroid peroxidase and antithyroglobulin antibodies), total immunoglobulin levels for IgG, IgM and IgA (gammopathies have been reported increased in association with autoimmune pregnancy loss) and nonspecific antibody evaluations for antiovarian and antiadrenal antibodies. All tests were performed utilizing commercially available assays.

Since determination of abnormal autoimmune function, in absence of well defined disease is difficult, the study, in consideration of the known high prevalence of abnormal autoimmunity in infertile women, actually attempted to determine absence of abnormal autoimmune function in study subjects. Minimum criteria for determination of abnormal autoimmune function were, therefore, on purpose set low. Presence or absence of autoantibodies (or other immune abnormalities), therefore, are not diagnostic of outright abnormal (auto)immune function. Diagnosis of autoimmunity should, therefore, be seen within such a context and is better described as “autoimmune noise,” rather than evidence of outright abnormal autoimmune function. A patient was considered autoimmune if she demonstrated in above described immune profile one or more positive laboratory abnormalities. In contrast, only women with not even a single autoimmune abnormality were considered non-immune.

Ovarian reserve assessments was performed utilizing baseline follicle stimulating hormone (FSH) and estradiol, utilizing an automated chemiluminescence system (ACS:180®, Bayer Health Care LLC, Tarrytown, N.Y.), as well as random antiMüllerian hormone (AMH) levels, utilizing a commercially available ELISA assay, as previously reported. Ovarian function parameters were then statistically correlated with number of triple CGG repeat counts on allele-2 (higher count allele). Based on previously published data, ≧35 triple repeats were considered the cut off, defining abnormally large repeat numbers in reference to ovarian function (i.e., normal range up to 34 repeats). The prevalence of abnormal triple CGG repeats in women with, and without, autoimmune abnormalities was then compared.

To prevent statistical distortions from small numbers of very high triple repeat numbers, associated with premutations and full fragile X mutations, the study was restricted to a triple repeat range between 35 and 55, the upper limit of the so-called intermediate zone. Patients with premutations and full FMR1 mutations are, therefore, not included in this study.

The statistical analysis involved comparisons of means between autoimmune and non-immune groups with t-test and a logistic regression, adjusted for age and AMH as co-variants. Age was chosen as co-variant because age, of course, affects ovarian function. AMH, but not FSH, was chosen as second co-variant because we previously reported that AMH, but not FSH, statistically correlates with number of triple CGG repeats on the FMR1 gene between 35 and 55 repeats.

Statistical analyses were performed using SPSS Windows, standard version 15.0. Continuous variables are presented as mean±1SD.

Since this study involved only the retrospective review of medical records, a review by the center's institutional review board (IRB) was not required. All new patients at our center, at time of initial registration, sign an informed consent, which allows for such retroactive medical record reviews as long as the confidentiality of the patient's medical record remains protected. A confirmatory letter from the Chair of the IRB is available upon request.

### Results of Example 1

TABLE 1
Patient characteristics in autoimmune and non-immune patient groups

| | AUTOIMMUNE | NON-IMMUNE |
|---|---|---|
| Number | 60 | 59 |
| Mean Age (years) | 36.0 ± 4.8 | 36.8 ± 5.2 |
| FSH (mIU/ml) | 16.0 ± 26.0 | 13.6 ± 11.2 |
| Estradiol (pg/ml) | 52.5 ± 26.2 | 51.4 ± 26.8 |
| AMH (ng/ml) | 1.4 ± 2.0 | 1.0 ± 1.0 |
| Triple CGG repeats (n) | | |
| Allele-1 | 27.4 ± 4.4 | 28.5 ± 5.6 |
| Allele-2 | 31.3 ± 4.2¹ | 33.3 ± 5.6¹ |

¹p < 0.05; In addition, however, non-immune patients also included significantly more women with ≧35 triple CGG repeats (RR 4.0, 1.3-11.9; p < 0.01).

Table 1 summarizes the findings in both patient groups. As the table demonstrates, the groups were practically evenly split: 60 women (50.4%) did, and 59 (49.6%) did not demonstrate autoimmune abnormalities. Mean ages in both groups also did not differ, with autoimmune patients being 36.0±4.8, and non-immune patients 36.8±5.2 years old. Both groups also did not demonstrate significant differences in mean FSH levels (16.0±26.0 and 13.6±11.2 mIU/ml), mean estradiol levels (52.5±26.2 and 51.4±26.8 pg/ml) and mean AMH levels (1.4±2.0 and 1.0±1.0), though AMH were slightly higher in autoimmune patients, suggestive of a trend (p=0.19).

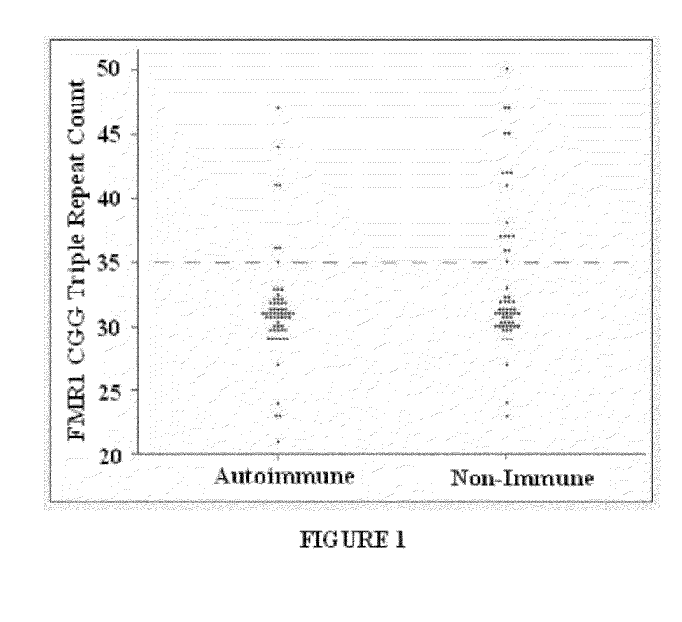

Triple CGG repeat expansion numbers on allele-1 also did not differ between both study groups, with autoimmune patients demonstrating 27±4.4 and non-immune women 28.5±5.6. Allele-2, however, did demonstrate a significantly higher mean triple repeat count in non-immune (33.3±5.6) than in autoimmune (31.3±4.2) patients (p<0.05). Non-immune patients also included significantly more women with 35, or more, triple repeats (RR 4.0, 1.3-11.9; p<0.01) (FIG. 1).

### Discussion of Example 1

That abnormal autoimmune function can lead to premature ovarian senescence in the form of POF has been known for decades. A possible association between abnormal autoimmune function and milder forms of premature ovarian aging has, however, previously not been made until we suggested such a possibility.

The association between abnormal autoimmune function and abnormal ovarian function—and, therefore, female infertility, has remained controversial. This fact was confirmed in this study, though, as previously noted, criteria for a positive diagnosis were purposefully set low. It, therefore, is important to reemphasize that patient selection in this study much better defines absence of abnormal autoimmune function than confirms presence of any (auto)immune abnormalities. The approximately 50% of patients (60/119) who were found to have at times very minimal evidence of autoimmune abnormalities can, therefore, not be automatically equated to previously reported infertile women with positive autoimmune findings, reported by Gleicher et al and Geva et al. For this reason, we describe the here reported immunological abnormalities as “autoimmune noise,” rather than formal evidence of abnormal autoimmune function.

Why infertile women would demonstrate a high than normal prevalence of abnormal autoimmune findings has so far remained unexplained. It is, however, noteworthy that animal models of abnormal autoimmune function have been reported associated with declining fertility and that female infertility in humans is associated with sub-clinical as well as clinical levels of abnormal autoimmunity. Despite widely expressed skepticism about an etiological association between abnormal autoimmune function and female infertility, POA, of course, is highly associated with female infertility. The here confirmed high prevalence of abnormal immune findings in POA patients further strengthens the arguments in favor of such an association. Indeed, this study suggests that the threshold for such effect on fertility may be rather low and that “immunological noise” levels of abnormal immune function may already be associated with increased risk for female infertility.

We have previously reported in a small pilot study that premature ovarian senescence in all of its grades of clinical severity, from POA to POF, is statistically associated with principally two known etiologies: genetic and autoimmune. Genetically, an increasing number of triple CGG repeats on the FMR1 gene correlate with prevalence and severity of ovarian insufficiency. This risk appears to start at, or mildly above, 31 repeats, a range currently widely considered normal. As a second, apparently independent etiology, “autoimmune noise” (and more severe forms of abnormal autoimmune function) also appears associated with risk for premature ovarian senescence in all of it forms. The independence of these two etiologies is supported by this study. Like the earlier pilot study, this paper demonstrate that genotypically these two patient groups significantly differ: autoimmune patients (in contrast to non-immune patients) demonstrate practically normal triple CGG repeats (mean 31.3±4.2; p<0.05) and fewer patients with ≧35 triple repeats p<0.001), the ultimate cut off chosen to define the upper limit of normal in regards to ovarian function.

The presence of even minimal laboratory evidence of abnormal autoimmune function thus appears to define in women with all levels of premature ovarian senescence, from POA to POF, a patient group which is distinct from women who experience premature ovarian senescence as a consequence of excessive triple CGG expansion repeats on the FMR1 gene. The observation of a trend towards higher AMH level in autoimmune patients is also supportive of an etiological distinction between these two groups and supports earlier observations in our pilot study. AMH appears to reflect ovarian reserve better than FSH and, therefore, not surprisingly, also correlates better with triple CGG counts on the FMR1 gene.

Like practically all autoimmune conditions, autoimmune-associated POA is clinically difficult to define. What constitutes abnormal autoimmune function in any given autoimmune condition varies and most are diagnostically defined by a panel of laboratory tests and/or clinical symptoms. Instead of defining what constitutes abnormal autoimmune function, we, therefore, in this study, decided to do the opposite: By setting a low standard for defining the presence of autoimmunity, and a high standard for absence of autoimmunity, we knowingly introduced bias against discovering a distinctive patient profile, characteristic of autoimmune POA. Choosing such a low standard for abnormal autoimmune function (i.e., “autoimmune noise”), potentially contaminated the autoimmune group with unaffected patients. The statistically distinct findings in the autoimmune POA group are, therefore, especially remarkable and the here utilized approach towards diagnosing abnormal autoimmune function reflects potentially, and respectively, weaknesses and strength of this study.

While exclusively describing infertility patients, this study has in two aspects special relevance to clinical practitioners: Clinically, it suggests that even minimally abnormal autoimmune function in younger women should raise the suspicion of risk towards POA. This means that in younger women with abnormal autoimmune function, periodic ovarian function assessments should become an integral part of long term autoimmune surveillance. Women, who are recognized to prematurely decline in their ovarian function, can then be properly counseled to pursue fertility preserving steps. On a more general basis, this study raises very basic questions about the clinical significance of “autoimmune noise.” Generally dismissed by rheumatologists and reproductive physiologists, alike, this study raises the question whether such low levels of abnormal autoimmunity may not, after all, have clinical relevance: If “autoimmune noise” can be relevant to reproduction, it may also affect other physiological processes.

The concept of a specific form of autoimmune POA are also further supported by a recent report of Tsigkou et al. These authors, amongst POF patients, were able to differentiate those with autoimmune etiology (from those with presumably mostly genetic etiologies) by demonstrating a very autoimmune-specific ovarian presentation, clinically characterized by high serum inhibin levels and, on pathology, by theca cell destruction in the presence of granulose cell survival. In analogy to here reported findings in POA, autoimmune POF, thus, appears etiologically and pathophysiologically distinct from other forms of POF, including the genetic form, associated with increased triple CGG expansion sizes on the FMR1 gene.

Theca cell destruction as a specific marker of autoimmune premature ovarian senescence may, indeed, explain observed differences in clinical presentation, such as at least a trend towards milder ovarian senescence in the autoimmune form, as reported here and also previously seen. Theca cell destruction will reduce androgen production by approximately 25 percent and may, therefore, be expected to lead to lower sex hormones than in women with other forms of premature ovarian senescence. An autoimmune etiology may, therefore, make such patients more responsive to androgen supplementation, which recently has been reported as successful treatment in selected women with POA. Proper clinical differentiation between autoimmune and genetic forms of POA may, therefore, become important for the selection of appropriate treatment options in women with prematurely diminished ovarian reserve.

### Example 2

This study investigated 55 consecutive infertility patients, undergoing ovulation induction for in vitro fertilization (IVF). Based on recent authoritative recommendations and a very high prevalence of prematurely diminished ovarian reserve in our patient population, our center, since January 2008, and after patients sign specific informed consents, includes the evaluation of triple CGG expansion sizes on the FMR1 gene in the initial evaluation of new infertility patients.

### Materials and Methods of Example 2

The tests were performed in accordance with published recommendations by commercial assay. The allele with lower triple repeats was designated allele-1 and the one with higher numbers as allele-2. Statistical associations were then calculated with allele-2, though only after correction for allele-1 counts. Comparisons of allele counts were done with two-sided tests, assuming equal variances with significance level 0.05. Tests were adjusted for pair wise comparisons within a row of each innermost suitables, using the Bonferroni correction.

Patients were then age-stratified, below (<) and at or above (≧) age 38 years. Moreover, based on prior investigations, demonstrating that a cut off at 34 CGG repeats discriminates between normal and risk towards diminished ovarian reserve 8, the study population was divided into women with less than (<) 35 and 35 or more (≧) triple repeats. Age 38 was chosen for the definition of a “younger” versus “older” population because age 37.5 years represents the approximate point where acceleration in female fertility decline commences.

For statistical analyses, univariate comparisons of baseline follicle stimulating hormone (FSH) on cycle days 2/3, baseline estradiol (days 2/3) and AMH (independent of cycle day) were then performed within strata of age and number of CGG repeats. A mixed model multivariate regression was used to assess the effects of allele-2 counts, continuously adjusted for average gonadotropin dosage administered during the stimulation cycle, and within age strata of < and ≧38 years of age.

FSH and estradiol measurements were done in house, while AMH assays were, as previously reported, run by commercial assay6.

All of the center's new patients sign at initial consultation an informed consent, which allows for the retroactive review of medical records for scientific (research) purposes without further permission, as long as the anonymity of the patient is maintained and confidentiality of the medical record is guaranteed. Both applied to this study and the study, therefore, based on the center's institutional review board's (IRB) policy, did not require further IRB review. A confirmatory letter from the IRB chairman is available upon request.

### Results of Example 2

TABLE 2
Patient characteristics in both patient groups

CGG TRIPLE REPEAT
EXPANSIONS COUNT*

| AGE | <35 (n = 41) | ≧35 (n = 14) |
|---|---|---|
| AGE (years) | 37.2 ± 4.5 | 37.4 ± 4.4 |
| <38 | 33.3 ± 3.2 | 33.9 ± 3.2 |
| ≧38 | 40.6 ± 1.9 | 40.9 ± 1.9 |
| FSH (mIU/ml) | 14.3 ± 14.8 | 14.3 ± 5.5 |
| <38 | 14.6 ± 18.4 | 15.4 ± 6.0 |
| ≧38 | 14.1 ± 11.1 | 13.1 ± 5.0 |
| Estradiol (pg/ml) | 56.2 ± 24.3 | 46.1 ± 23.2 |
| <38 | 57.3 ± 25.3 | 37.8 ± 12.1 |
| ≧38 | 55.1 ± 24.2 | 53.0 ± 28.8 |
| AMH (ng/ml) | 1.1 ± 1.7 | 0.6 ± 0.6 |
| <38¹ | 1.8 ± 2.3¹ | 0.6 ± 0.7 |
| ≧38 | 0.6 ± 0.5 | 0.6 ± 0.4 |
| Allele-1 | 26.2 ± 4.2 | 29.1 ± 4.3 |
| <38 | 26.3 ± 4.4 | 27.3 ± 5.0 |
| ≧38 | 26.1 ± 4.1 | 31.0 ± 2.7 |
| Allele-2¹ | 29.3 ± 2.7 | 39.7 ± 5.1 |
| <38¹ | 28.8 ± 3.0 | 38.7 ± 3.6 |
| ≧38¹ | 29.7 ± 2.5 | 40.7 ± 6.4 |
| Gonadotropin dosage (IU) | | |
| <38¹ | 4,716 ± 2,233¹ | 6,718 ± 2,597 |
| ≧38 | 6,181 ± 1,956 | 6,918 ± 1,707 |
| Oocyte retrieved (n) | | |
| <38² | 10.1 ± 5.6¹ | 3.1 ± 2.3 |
| ≧38 | 4.9 ± 4.3 | 5.9 ± 4.9 |

*All data sets are reported as mean ± 1 standard deviation
¹Denotes a statistically significant difference (p < 0.05) between groups in same line
²Denotes a statistical significance of p = 0.006

Table 2 summarizes patient characteristics in both patient groups. Neither FSH, nor estradiol differed significantly in either women with <35 or ≧35 CGG repeats, and whether they were below or above age 38 years. Women with <35 CGG repeats, however, demonstrated significantly lower AMH levels at age ≧38 years (0.6±0.5 ng/ml) than <38 years of age (1.8±2.3 ng/ml; p<0.05).

Below age 38, AMH levels were also significantly higher in women <35 versus ≧35 repeats, while ≧age 38 AMH levels no longer reflected such differences. Indeed, in women ≧age 38 years, AMH levels also did not differ between those with <35 and ≧35 repeats. In summary, AMH levels, thus, reflected triple CGG expansions in younger women (<38 years) with normal triple CGG counts (<35) but lost correlation in older women (≧38 years) and women with excessive CGG expansion sizes (≧35).

Gonadotropin dosages also correlated with AMH levels with respect to the number of CGG repeats. Women with normal CGG expansion sizes (<35) used significantly less gonadotropin if they were younger (<38 years; 4,716±2,233 IU) than older (≧38 years; 6,181±1,956 IU; p<0.05) and younger women with <35 CGG repeats used less gonadotropins than their older counterparts (p<0.05). In contrast, there was no difference in gonadotropin utilization in younger (<38 years; 6,718±2,597 IU) and older women (≧38 6,918±1,707 IU) if they demonstrated excessive (≧35) CGG repeats.

Younger women, under age 38, with <35 CGG repeats, also produced significantly more oocytes (10.1±5.6) than younger women with ≧35 repeats (3.1±2.3; p=0.006) and older women ≧38 years of age (p<0.05). Once again, older women (≧38 years) did not show this difference (4.9±4.4 vs. 5.9±4.9 oocytes).

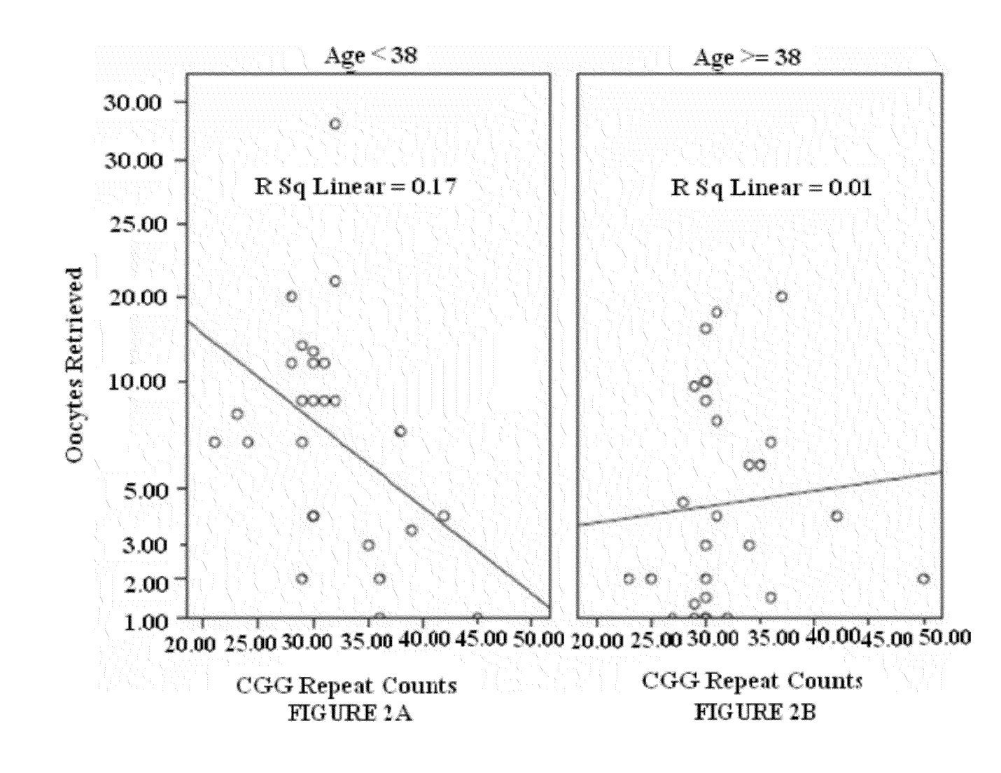

In the multivariate model, a significant interaction of CGG triple repeat counts on allele-2 with age was noted (p=0.04). Since age 37.5 years has been reported to represent accelerated decline in female fertility¹¹, we chose to perform the remaining analysis in two different age strata, adjusted for gonadotropin dosage as continuous covariates. When this was done, the number of oocytes retrieved in younger women (<age 38) was inversely related to the number of CGG repeats (df=1; F=5.1; p=0.03). In contrast, no such relationship was present in the strata of older women (≧38 years) (FIG. 2A and FIG. 2B). In other words, as shown in FIG. 2A and FIG. 2B, the number of triple CGG expansions on the FMR1 gene significantly correlated with oocyte yield in younger women below age 38 (FIG. 2A), but not in older women ≧38 years of age (FIG. 2B).

### Discussion of Example 2

The data demonstrates for the first time a direct statistical association between number of triple CGG expansions on the FMR1 gene and specific ovarian reserve and fertility parameters. Gonadotropin dosages usually increase with decreasing ovarian reserve, though whether such increases in stimulation improve oocyte yield has remained controversial. Oocyte yield, itself, is, however, quite likely the most accurate currently available clinical reflection of ovarian reserve.

By demonstrating that the number of CGG repeats in young women with what are widely considered basically normal triple CGG expansion sizes (up to 55 repeats) statistically correlates to number of retrieved oocytes, this study further strengthens the previously reported statistical association between triple CGG numbers and ovarian reserve. In these previous studies, ovarian reserve was, however, only indirectly assessed through the laboratory parameters FSH and AMH. In this study, we were able to take this correlation one step further by demonstrating that the correlation between triple CGG expansion numbers also extends to the number of retrieved oocytes and amounts of gonadotropin utilized.

Here reported findings that gonadotropin dosages and oocyte yields differ in younger and older women, and based on, in reference to ovarian function considered normal (<35) and elevated (≧35) triple CGG repeat counts, should not surprise. Above age 38, prevalence of diminished ovarian reserve will progressively rise with age. Similarly, above 35 CGG repeats risk for premature ovarian senescence, and therefore diminished ovarian reserve, progressively increases. Increasing gonadotropin utilization and decreasing oocyte yield are, therefore, expected beyond age 38 years and with triple CGG numbers beyond 35. At most advanced ages, and with more excessive CGG repeat numbers, practically all women can be expected to suffer from degrees of diminished ovarian reserve. Here reported findings, therefore, perfectly complement previous reports, demonstrating a direct statistical association between triple CGG expansions sizes and ovarian function.

This study also comments on another potentially very important question: Whether administration of increasing gonadotropin dosages concomitantly increases oocyte yields in women with diminished ovarian reserve has remained highly controversial. Out and Thomas, supported by recent data from our own center, suggested that, in contrast to older patients, younger women may, indeed, benefit from increasing gonadotropin dosages. The here presented data, however, quite clearly demonstrate, that even younger women (<38 years) with abnormally high triple repeat expansions (≧35), despite high dosage gonadotropin stimulation, still produce only relatively very low oocyte yields.

Despite routine use of maximal gonadotropin dosages (daily gonadotropin 450-600 IU), they in this study produced only a meager mean of 3.1±2.3 oocytes, while women of the same young age, with normal triple CGG counts (<35), produced a significantly larger number of 10.1±5.6 eggs (p=0.006). Mean gonadotropin dosages between these two groups, in view of identical stimulation protocols, did not differ (data not shown).

Since FMR1 information at time of treatments was unknown, and since gonadotropin dosages were identical in women with normal and elevated triple CGG repeats, these observations suggests potentially important conclusions: As Out and Thomas' and our data support in younger women benefits on oocyte yields from higher gonadotropin dosages, and since this study in younger women with abnormally high triple CGG repeats fails to confirm this, we conclude that younger women with ovarian resistance to stimulation fall into two distinct sub-groups: Those with abnormally high triple CGG counts, even with higher gonadotropin dosages, will remain resistant. By definition this, however, establishes a second group of younger women with normal CGG counts below 35, which can be expected to be responsive to higher gonadotropin dosages.

In this context we previously reported two primary, and independent, etiologies for premature ovarian senescence: a genetic, defined by excessive triple CGG repeats on the FMR1 gene, and an autoimmune etiology, defined by very non-specific evidence of abnormal autoimmune function. We furthermore demonstrated that both of these patient populations represent distinct phenotypes and genotypes, and predicted that they may respond differently to fertility treatments, including ovarian stimulation.

The here presented data now suggest that autoimmune patients may represent those women who, despite obvious reduction in ovarian reserve, still are capable of improving oocyte yields with higher gonadotropin stimulation. If confirmed, this would suggest that the genetic form of premature ovarian senescence behaves more like physiological aging, while the autoimmune form may lend itself better to therapeutic interventions.

Such a distinction between genetic and autoimmune forms of premature ovarian senescence is also supported by very recent data from Tsigkou et al, who reported that high serum inhibin concentrations are able to discriminate between autoimmune and other forms of primary ovarian insufficiency. They further concluded that women with autoimmune etiology demonstrate increased inhibin production and selective theca destruction with initial preservation of granulose cells. Such selective theca destruction, of course, would reduce ovarian androgen production, the substrate for aromatization in granulose cell, reflecting approximately 25 percent of a female's overall androgen production.

Women with the autoimmune form of premature ovarian senescence, therefore, may be expected to be more sex-hormone deficient than their counterparts with a genetic FMR1-based etiology and, therefore, also more responsive to androgen supplementation. This observation, in turn, may explain why selected women with premature ovarian senescence respond well to supplementation with the androgen dehyroepiandrosterone (DHEA), while others do not. Hypothesizing further, this would suggest that women with genetic premature ovarian senescence would not benefit from either DHEA supplementation or higher gonadotropin dosages, while those with the autoimmune form of the condition should benefit from both.

This study ultimately raises the question who should be tested? While a final answer to this question, of course, awaits further confirmatory studies, the here presented data suggest at the present time at least two potential target populations: A first one is represented by women with abnormally elevated FSH levels (or other ovarian reserve tests), as also suggested by other authoritative sources. We would add to this recommendation that FSH levels (and other ovarian function tests) should be assessed in age-specific ways, which allows for the much earlier recognition of premature ovarian senescence than standard testing with universal cut off levels. A second obvious target group would be women with increased risk towards premature ovarian senescence, such as patients with a maternal family history of early menopause or women exposed to ovario-toxic agents (i.e., radio- and chemo-therapies).

Looking even further into the future, one, in view of the suspected high prevalence of women with premature ovarian senescence amongst infertility patients, can expect FMR1 testing to become a routine infertility test and, indeed, possibly, even a routine screening test for young women, just considering their reproductive future.

### Example 3

This study expands and confirms our earlier pilot and adds to the understanding of reported triple CGG repeats in the general population and amongst women with infertility. Though up to 45 CGG repeats are considered normal, Fu et al reported that most humans demonstrate between 29 and 30 repeats (FIG. 3). Their distribution curve, therefore, demonstrates a very large peak at these triple CGG numbers, with much smaller patient populations to the right (higher counts) and to the left (lower counts). From a statistical point it is, therefore, tempting to consider individuals with equal or less than 28, and 31 or more, triple repeats, outside of an expected “standard” range. Assuming, once more, that excessive triple CGG numbers reflect risk towards premature decline in ovarian reserve, distributions to the right of above noted peak may, therefore, represent at least a portion of those approximately 10 percent of all females who have been suggested to suffer from premature ovarian senescence—a prevalence even much higher amongst infertile women.

Assuming all of this to be correct, then infertile women may be expected to demonstrate a CGG triple repeat pattern which in comparison to the general population is moved towards the right (higher repeat numbers). This study, therefore, also investigated the distribution pattern of CGG repeats on the FMR1 gene in infertile women and compared it statistically with the distribution pattern, previously reported by Fu et al for the general population.

To avoid statistical confusion from excessively high CGG count outliers, we in this study concentrated only on counts between 35 and 50 CGG repeats, the upper two thirds of what currently is considered normal, and the lower half of the so-called intermediate (“gray”) zone. Lower triple CGG ranges have also been reported to demonstrate more linear correlations with ovarian function than higher ranges. We are also utilizing in this manuscript the previously coined acronym premature ovarian aging (POA) for milder forms of premature ovarian senescence (in differentiation from POF) which differs in some aspects from normal physiologically aging. For example, POA patients do not demonstrate increased embryo aneuploidy, as is associated with physiologic female aging.

### Materials and Methods of Example 3

This is a cross-sectional study of a convenience sample of female infertility patients, attending an infertility center. Following recently published professional guidelines, the center since January 2007, expanded indications for FMR1 (fragile X) gene investigations. In view of the previously reported high prevalence of POA in our patient population, characterized by laboratory evidence for premature ovarian senescence, such as prematurely elevated FSH and reduced AMH levels, fragile X testing has now become routine.

POA, as previously reported in detail, is a program-specific diagnosis, which defines patients to suffer from prematurely diminished ovarian reserve if their baseline FSH level exceeds the 95 percent confidence interval of an age specific, infertile population. The clinical validity of this diagnosis was confirmed by demonstrating that, within each age group, POA patients produce significantly diminished oocyte numbers in response to stimulation with gonadotropins. In the here reported study population, up to age 38 years, 30 percent demonstrated baseline FSH levels above 12 mIU/ml and 40 percent AMH levels below 1.0 ng/ml, while above age 38, 43 percent demonstrated FSH levels above 12 mIU and 80 percent AMH levels below 1.0 ng/ml. The here reported infertility population, thus, to a considerable degree suffered from diminished ovarian reserve.

The distribution of CGG triple repeats in a general population was reported by Fu et al (FIG. 3). Their distribution data were used for comparison to 158 patients in this study of infertile women (316 FMR1 alleles). Since females were a significant part of Fu's study population, one has to assume up to approximately 10 percent POA patients amongst them. Their reported distribution pattern in a normal population may, therefore, be somewhat biased towards higher triple repeats. Further deviation towards higher triple CGG expansion numbers by our study population would, therefore, support a statistical association between increasing triple repeat expansion numbers and infertility risk.

In 11 months, 158 consecutive, still spontaneously cycling, female infertility patients underwent FMR1 gene evaluations of 316 alleles. Since most normal individuals demonstrate 29 to 30 CGG repeats, we arbitrarily for purpose of this study, and consistent with prior publications chose to define up to 35 repeats as normal (standard) distribution. We then broke the study population into three groups women in considered normal range with less than 35 repeats (n=122), representing 77.2 percent of patients (Group I), a primary study group (Group II) with 35 to 50 repeats (n=30), representing 19.0 percent of patients, and Group III (n=6), or 3.8 percent of the population, with in excess of 50 repeats.

While Fu et al considered both alleles in each patient in their statistical assessments of triple CGG distributions, we for consistency chose to concentrate on the higher count allele for analysis, though corrected statistically for the lower allele count. The allele with fewer CGG repeats was designated allele-1, and the one with more repeats as allele-2. All statistical assessments were made based on allele-2's associations with indicators of ovarian reserve, defined by FSH and AMH levels, and thyroid function, defined by thyroid stimulating hormone (TSH) levels. FSH levels were uniformly obtained on days 2/3 of menstrual cycle, while AMH and TSH levels were drawn at random, as they do not demonstrate cycle specific changes. Only highest FSH and TSH, and lowest AMH levels were considered for statistical evaluation. FMR1 gene analysis was performed on blood samples by commercial assays (Genzyme Analytical Services, Westborough, Mass.; Quest Diagnostics, Lyndhurst, N.J.; and LabCorp, Burlington, N.C.), following recommended methodologies. AMH levels were also obtained by commercial assay (Quest Diagnostics, Lyndhurst, N.J.; LabCorp, Burlington, N.C.), utilizing an ELISA assay (MIS/AMH ELISA DSL-10-14400), while FSH and TSH were assayed in house, using the Automated Chemiluminescence System (ACS:180®, Bayer Health Care LLC, Tarrytown, N.Y., USA). Only patients with results in assay ranges were considered for statistical evaluation.

We then constructed a series of multivariable regression models to test for possible statistical associations between increasing CGG triple repeats in allele-2 and ovarian (FSH and AMH), as well as thyroid (TSH), function parameters, adjusting the model for female age and number of triple repeat expansions on allele-1. The model was initially investigated for the total patient population and then, separately, for Groups 1, 2 and 3.

All variables were log-converted, to adjust for normality. Variables were included in this model based on specific theoretical considerations: Since age is a major factor in regard to ovarian function, all models were adjusted for age. When groups were stratified by age category, age was maintained as a continuous covariate within models. Similarly, CGG counts in allele-1 were included in all tested models. All models were also tested for interactions, and checked for residual analysis of the regression. Statistical analysis was performed using SPSS version 15.0. Continuous values are presented as mean±standard deviation (SD).

At initial consultation at the center, all patients sign an informed consent, which permits the review of their medical records for study purposes, as long as the patients' anonymity is maintained. Studies involving only chart review are, therefore, exempt from review by the center's institutional review board (IRB). A letter of confirmation from the Chair of the IRB is available, upon request.

### Results of Example 3

By December 2007, 158 consecutive female infertility patients had undergone evaluations of their FMR1 gene. FIG. 3 demonstrates the distribution of triple CGG repeat numbers on allele-1 (interrupted line) and allele-2 (solid line) against a shaded background, represented by the distribution curve reported by Fu et al for a general population. As can be seen, even the distribution of triple repeats on allele-1 (the lower count allele) is mildly shifted to the right from the distribution of Fu's unselected patient population (including both allele counts). In contrast, triple repeats on allele-2 are quite obviously shifted to towards higher triple repeats. One hundred-twenty two infertile women (77.2%) demonstrated less than 35 triple repeats (Group 1), 30 (19.0%) showed 35 to 50 repeats (Group 2) and only 6 (3.8%) above 50 triple CGG repeats (Group 3).

TABLE 3
Patient characteristics in groups 1-3 (mean ± SD)

| | Group 1 | | Group 2 | | Group 3 |
|---|---|---|---|---|---|
| CGG repeats | ≦34 | | 35-50 | | ≧51 |
| (n) | (122) | | (35) | | (6) |
| Ethnicity (n/%) | | | | | |
| Caucasian | 86 | (70.5) | 20 | (57.1) | 6 (100) |
| African | 13 | (10.7) | 2 | (5.7) | 0 |
| Asian | 23 | (18.9) | 8 | (22.9) | 0 |

| | | | | |
|---|---|---|---|---|
| Other | 0 | 5 | (14.3) | 0 |

| | | | |
|---|---|---|---|
| Age | 36.3 ± 5.7 | 36.1 ± 4.5 | 35.0 ± 7.2 |
| FSH (mIU/ml) | 16.8 ± 22.3 | 13.5 ± 6.8 | 38.3 ± 52.1 |
| Estradiol (pg/ml) | 66.1 ± 90.6 | 51.2 ± 23.9 | 43.3 ± 16.0 |
| AMH (ng/ml) | 1.4 ± 1.9 | 1.1 ± 1.2 | 6.2 ± 11.9 |
| TSH (μIU/ml) | 2.1 ± 1.8 | 1.6 ± 1.0 | 1.9 ± 1.5 |
| FMR1 gene | | | |
| Allele-1 | 27.4 ± 3.9 | 29.6 ± 3.2 | 28.5 ± 4.3 |
| Allele-2 | 30.1 ± 2.1 | 39.6 ± 4.0 | 71.5 ± 15.0 |

Patient groups did not differ significantly from each other in any of the listed parameters.

Table 3 summarizes patient characteristics in all three study groups. After log conversion, no statistical differences were apparent in either race, age, FSH, baseline estradiol, TSH or AMH levels. Trends, primarily observed in Group 3, probably did not reach significance because of the very small sample size in women with over 50 CGG repeats.

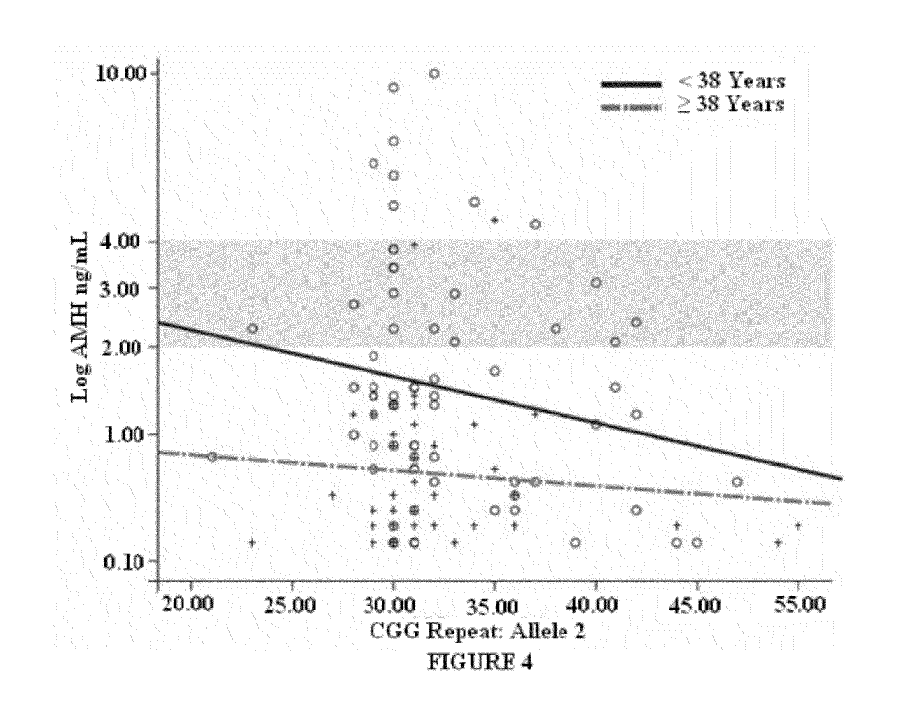

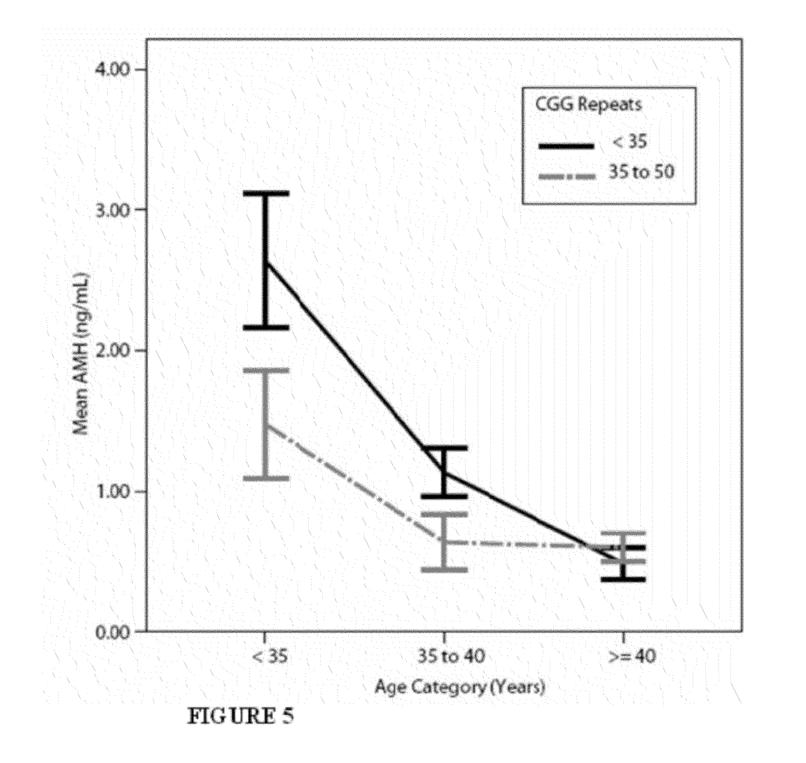

As expected, allele-1 triple repeat expansion numbers were almost identical between all three groups, while mean levels for allele-2 increased from 30.1±2.1 in Group 1 to 39.6±4.0 in group 2 and 71.5±15.0 in group 3 (p<0.001). Over the entire triple repeat spectrum, involving all three patient groups, there was no statistical correlation between triple repeat numbers on allele-2 and FSH [β=0.03, t(91)=0.26, p=0.56]. Increasing triple repeat numbers were, however, over the entire spectrum [β=−0.24, t(104)=−2.7, p=0.008] statistically correlate to AMH levels, a finding, as FIG. 4 demonstrates, in principle only reflective of younger women, under age 38 years. As FIG. 5 demonstrates, AMH levels were at all ages (until age 40, when practically every woman suffers from diminished ovarian function) lower in women with 35 or more CGG repeats (Groups 2) than in women with less than 35 repeats (Group 1) [F (1.87)=5.3, p=0.025].

Group 1 patients demonstrated a mean age of 36.3±5.7 years and clearly abnormal ovarian function, with a mean FSH value of 16.8±22.3 mIU/ml and AMH of 1.4±1.9 ng/ml (Table 3). Neither FSH, nor AMH levels demonstrated, however, evidence of correlation to the number of triple CGG repeats. As expected, FSH correlated positively (R=0.33, p=0.045) and AMH negatively (R=−0.53, p<0.001) to patient age.

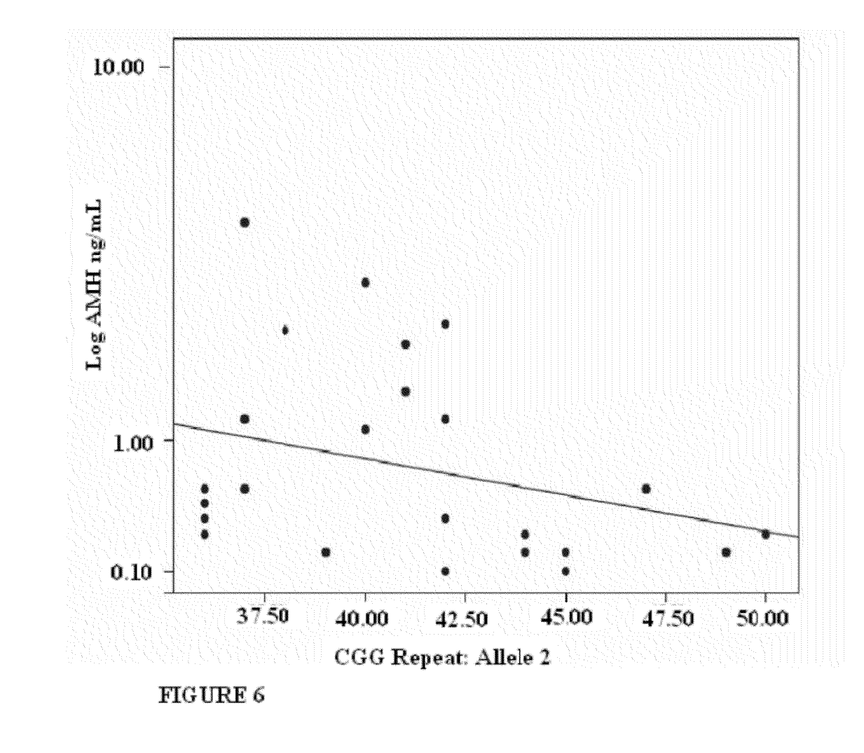

Group 2 patients demonstrated a mean age of 36.1±4.5 years. FSH levels, at 13.5±6.8 mIU/ml, were high and AMH levels were low at 1.1±1.2 ng/ml. Within this group, FSH levels also did not correlate with triple repeat expansion numbers; however, AMH levels did to a significant degree (FIG. 6; R=−0.41, p<0.013).

The number of Group 3 patients was too small for further statistical evaluation.

### Discussion of Example 3

Excessive triple repeat CGG counts on the FMR1 gene increase risk for POF is well established. More recently, milder stages of premature ovarian senescence, often just characterized by laboratory abnormalities, such as baseline FSH elevations, have also statistically been linked to abnormal increases in triple CGG repeat numbers. Such milder ovarian function abnormalities have, like POF, mostly been associated with premutations but, at times, also with lower CGG repeat numbers in the so-called intermediate (“gray”) zone, and at higher levels of repeats within what is considered normal range.

Because premutation range triple repeats within one generation can expand to full mutations, professional organizations recently have recommended a more proactive approach towards preemptive fragile X testing. The motivations for these recommendations were, however, primarily of genetic nature. Timely maternal testing of women with premutation range CGG repeats should improve detection of women at risk for giving birth to offspring with full FMR1 mutations. Increased fragile X testing was, therefore, not intended to in any way affect, or improve, the diagnosis of female infertility.

We were, however, intrigued by the possible diagnostic value of triple CGG expansion numbers for female infertility. Two recent reports had suggested that even CGG repeat levels below the premutation range may already denote risk for premature ovarian senescence. Premature diminution in ovarian reserve, in turn, will quite obviously result in reduced female fertility. Infertile women should, therefore, demonstrate a shift towards higher triple CGG expansion numbers in comparison to normal controls.

Even though expert panels very recently still questioned the association between triple repeats below premutation range and premature ovarian senescence, a small pilot study convinced us that triple repeat expansion numbers may, indeed, be reflective of ovarian reserve and, therefore, be useful as diagnostic tools in infertility.

Demonstrating that infertile women, in comparison to controls, demonstrate a shift towards higher triple repeat expansion numbers would offer a first level of confirmation. This study, indeed, did that by confirming that infertile women under treatment very clearly demonstrate elevated triple repeat expansion numbers in comparison to the general population (FIG. 3).

Statistical comparisons to historical controls always have to be viewed with caution. The format of statistical analysis utilized in this study should, however, be reassuring: While differences in assay technology, and therefore results, between Fu's and our studies cannot be ruled out, we on purpose analyzed both alleles separately in our study since a combined analysis, as performed by Fu et al can obfuscate findings if results from both alleles balance each other out. It is actually quite remarkable how allele-1 (lower count allele) in our study almost perfectly matches the distribution curve, reported by Fu et al (for both alleles) for a general population. Since allele-1 in our study represented uniformly the lower count allele, one, of course, would have expected this allele count to present to the left of Fu's curve. The fact that this is not the case suggests a right shift for allele-1.

FIG. 3 also demonstrates a very convincing right shift towards higher triple CGG numbers on allele-2. Such a shift can, of course, by definition be expected, considering that allele-2 represents only higher count alleles. The fact that both alleles are, however, to the right side of expectation in comparison to Fu's curve, strongly suggests that infertility patients, indeed, demonstrate a right shift towards higher triple CGG counts from normal average individuals.

We next investigated the statistical correlation between CGG triple repeats and very specific parameters, reflective of ovarian reserve. While FSH levels did not correlate with triple CGG numbers to a statistically significant degree, AMH, in contrast, demonstrated such a statistically significant correlation throughout the entire spectrum of triple repeats (p=0.008). As FIG. 4, however, demonstrates, this correlation was primarily due to findings in younger women, under age 38 years, since older women did not demonstrate any correlation.

This finding, of course, should not surprise and, indeed, be expected. As FIG. 4 demonstrates, in a population with already low AMH levels due to a high prevalence of premature ovarian senescence, advanced female age, of course, further suppresses AMH levels, often reducing them to undetectable levels. The inverse correlation between triple CGG repeats and AMH levels is further demonstrated by the fact that Group 1 women with less than 35 repeats until age 40 demonstrate significantly higher AMH levels than Group 2 patients (p=0.025; FIG. 5). Even in Group 2 patients, women with 35 to 50 CGG repeats, and for the purpose of this study considered outside of the “standard” range, our data suggest a statistical correlation with AMH (though again not with FSH) (FIG. 6).

That triple repeat numbers in this range correlate with AMH, but not FSH, also does not come unexpected. AMH, of course, is representative of small, up to four millimeter, growing follicles (up to antral stage). The number of follicles remaining in ovaries has, in turn, suggested to be the most reliable indicator of “ovarian age”, and whether ovaries age physiologically or prematurely, both reduce available follicles for monthly recruitment. AMH biochemically represents early stage follicles during the recruitment process better than FSH.

Our observations may also help explain how the FMR1 gene may affect the (premature) ovarian aging process: With increasing CGG expansion sizes (up to full mutation) the transcription of the message of the gene product (the so-called fragile X mental retardation protein, FXMRP) seems to increase. Adult onset fragile X-associated tremor/ataxia syndrome (FXTAS) (and related animal models), a neurodegenerative condition primarily seen in male fragile X premutation carriers, suggest that increasing transcription (i.e., mRNA) of FXMRP may correlate to disease risks.

In analogy, Sullivan et al suggested that premature ovarian senescence may represent the adult onset consequence of excessive FXMRP transcription in the female. Such an assumption, of course, presupposes an effect of FXMRP on ovarian function, which so far has not been established. FXMRP has, however, been suggested to affect germ cell proliferation in women and men. An effect on ovarian function would, therefore, not surprise.

Specific triple CGG count ranges (i.e., premutations and full mutations) have previously been statistically correlated with specific ovarian dysfunction diagnoses [i.e., increased FSH, irregular menses, early menopause, and POF]. We in this study, however, for the first time, attempted to statistically correlate specific triple CGG repeat numbers with specific ovarian function/reserve parameters (i.e., FSH, AMH). While low level triple repeats (including expansions in the intermediate zone) do not denote risk for (single generation) expansion to full fragile X mutations, in regards to female infertility, they potentially represent the most interesting and relevant expansion ranges.

At high triple repeat expansion numbers, all forms of premature ovarian senescence, including POA and POF, are widely expected, clinically usually very obvious and, therefore, relatively simple to diagnose. A diagnosis of impaired ovarian function and/or female infertility is, however, much more difficult to make when the diagnosis is less obvious and more unexpected. This is the case at lower expansion ranges, up to approximately 50 CGG repeats, where traditional tests of ovarian reserve are not yet necessarily very obviously abnormal and only age-specific ovarian function testing often allows for a timely diagnosis.

In analogy to FXTAS, correlations between number of triple repeat expansions in this range and ovarian function reserve appear quantitative as well as qualitative. As expansion ranges increase, ovarian senescence not only becomes more likely, but also more severe and, therefore, easier to diagnose, though once triple repeats reach the premutation range (55-200 repeats), the correlation no longer appears to remain linear.

Indeed, a mid-range between approximately 80 to approximately 100 repeats appears clinically most symptomatic. At lower (outside the “standard” range) CGG repeat numbers, premature ovarian senescence will be milder, more difficult to diagnose and, therefore, frequently overlooked. A diagnostic test, defining risk for premature ovarian senescence and, possibly, also improving the diagnosis of infertility, would, therefore, be very welcome.

In summary, this study demonstrates for the first time a direct association between female infertility and elevated triple CGG repeat expansion numbers on the FMR1 gene. Risk for premature ovarian senescence, and infertility, already starts at triple repeat numbers, currently considered entirely normal. Triple repeat numbers can be used to denote future risk for premature ovarian senescence, reach a more timely diagnosis of POA, and avoid the pseudo diagnosis of so-called unexplained infertility, currently still representing approximately 20 to 30 percent of all infertility diagnoses. More importantly, FMR1 testing will allow young women to prospectively view their reproductive future, and to take fertility preserving steps, if evidence for early premature ovarian senescence can be confirmed.

### Example 4

The FMR1 (fragile X) gene is increasingly investigated. In females abnormally high CGG numbers in so-called premutation range (55-200 repeats) has for long been known statistically associated with premature ovarian failure (POF), frequently also called premature menopause. More recently, we and other investigators have demonstrated that an increased risk towards premature ovarian aging (POA), sometimes in milder degrees than outright POF, also exists at lower CGG counts and that CGG counts statistically, indeed, correlate with risk towards POA.

Medical literature suggests that fertility parameters can vary between races/ethnicities. This is best reflected by widely reported lower in vitro fertilization (IVF) pregnancy rates in Chinese than Caucasian women. We also previously demonstrated that Chinese carry many times the risk of Caucasian women in being affected by POA.

It, therefore, is not surprising that a recent Conference on the FMR1 gene, held by the National Institutes of Health in the U.S.A., suggested that a comparative investigation of CGG repeat patterns on the FMR1 gene in various races would be desirable. As such, we performed a study with the objective of determining whether CGG counts differ between different races/ethnicities. During this investigation, we determined that 26-34 CGG repeats of the FMR1 gene reflect normal ovarian reserve.

More specifically, we performed a cross-sectional cohort study that investigated 385 females (344 infertile women and 41 oocyte donors), the numbers of CGG repeats on the FMR1 gene and differences between races/ethnicities. Traditional definitions of neuropsychiatric risks are classified as common, intermediate, premutation and full mutation ranges. Normal CGG count range was here, however, defined by box and whisker plot as 26-34 repeats (median 30). Distribution of abnormal outliers in CGG counts from this normal range was then compared between women of Caucasian, African and Asian descent. African and Asian women demonstrated a higher prevalence of two normal count alleles (65%) than Caucasians (54.3%; P=0.03). Caucasians demonstrated the highest rate of allele abnormalities (43.3%) and were the only race/ethnicity also demonstrating abnormalities in both FMR1 alleles. Asian women demonstrated significantly fewer low outlier counts than Caucasians (P=0.002) and Africans (P=0.03). This study, thus, suggests significant racial/ethnic differences in triple CGG counts on the FMR1 gene between races/ethnicities. Since CGG counts on FMR1 are associated with ovarian reserve, these findings may reflect potential differences between races/ethnicities in ovarian function and female fertility reported in the literature.

### Materials and Methods of Example 4

We performed a cross sectional study of 385 females (770 FMR1 alleles), presenting to our center during 2008. Except for a small minority (n=41) of oocyte donors, all were infertility patients. Respective races/ethnicities were accepted as self-reported.

Because of a very high prevalence of premature ovarian senescence (Barad et al., 2007), our center, based on authoritative recommendations (Wittenberger et al., 2007), since January 2008 routinely performs fragile X testing with special written informed consent.

Such testing follows standard laboratory procedures and has been previously described in detail (Gleicher et al., 2009a) and utilizes at random commercial assays (Genzyme Analytical Services, Westborough, Mass.; Quest Diagnostics, Lydnhurst, N.J.; LabCorp, Burlington, N.C.). Briefly, the number of triple CGG repeats on both alleles of the FMR1 gene is determined by testing DNA by Southern blot and polymerase chain reaction (PCR) to determine size and methylation status of the CGG repeats. Southeren blot analysis was performed with the probe St.B12.3 on ecoRland Eagle digested DNA. PCR products were generated using fluorescent labeled primer and sized by capillary electrophoresis. The allele with lower count is designated as allele-1 and the one with higher count as allele-2.

In the first stage of this study we simply compared triple CGG counts on both alleles between Caucasian, African (including patients of African, Caribbean and U.S. descent) and Asian women. Since patients identified themselves as Hispanics amongst Caucasian women, females of obviously African inheritance and even amongst women with Asian ethnicity, Hispanics were not identified as a separate group. Asian women represented in approximately 95% of cases patients of Chinese descent. The remainder included small numbers of Japanese, That (often also of Chinese ethnicity), Vietnamese, Cambodian, Philippino, Indian and Pakistani women.

As initially reported by Fu et al, a large majority of individuals in the average population presents with 29 to 30 triple repeats (Fu et al., 1991). When the distribution curve for CGG repeats is drawn, this is reflected in a dominant spike at these numbers of repeats, with relatively smaller populations to the right and left of this spike. These population groupings on both sides of the spike of 29-30 CGG repeats have previously been shown at risk towards premature ovarian senescence, with higher counts denoting marginally higher risk than lower counts (Gleicher et al., 2009c).

For purpose of this study we, however, did not want, as in past studies, to rely on arbitrarily chosen “normal” triple CGG counts, based on the population studies by Fu et al (Fu et al., 1991). We, therefore, utilized a new statistical format of data analysis, which is widely used to define cut offs for normal laboratory findings in general populations:

We constructed box and whisker plots of triple CGG repeat counts on both alleles for the whole study population and for individual racial/ethnic groups.

Box and whisker plots are characterized by establishing a “box,” which contains the middle 25^{th}-75^{th}% percentile, representing 50% of all cases. Then, in addition, so-called “outside” values are established, defined as smaller than the lower quartile minus 1.5-times the so-called interquartile range or larger than the upper quartile plus 1.5 times the interquartile range (Tukey J, 1977). This process defined 26 to 34 CGG repeats as the population's presumptively normal CGG count, with a median of 30 repeats.

Remarkably, these data coincide closely with the previously noted distribution peak of 29 to 30 repeats, reported by Fu et al (Fu et al., 1991).

Study subjects were then classified into four groups: Group 1 included women with both alleles in normal range of 26 to 34 CGG repeats; Group 2 included women with allele-1 in abnormally low (≦25 repeats) and allele-2 in normal ranges; Group 3 represented subjects with allele-1 in normal and allele-2 in high abnormal ranges (≧33 repeats); and Group 4 involved women with allele-1 in abnormal low and allele-2 in abnormal high ranges (i.e., both alleles were thus in abnormal range; No cases were in this study encountered with both alleles abnormally low, a potential Group 5 or abnormally high, potentially Group 6, though such combinations should theoretically occur). Membership in these four groups was then compared amongst the various racial/ethnic populations. Because, except for a small number of oocyte donors, most study subjects were infertile, here reported cut off values and findings are not necessarily reflective of normal, random patient populations.

Continuous variables were tested for normality with the Kolmogorov-Smirnov test. Normally distributed variables are shown as means±standard deviation (SD), while Variables, corrected for normality by log conversion, are presented as geometric means and 95% confidence intervals (CI) of the mean. Rates are presented as raw numbers and percentages.

Data analysis was performed using SPSS for windows, version 15.0. Demographic and biochemical data were analyzed by analysis of variance, Chi square or Fisher's exact test to compare frequencies. Differences were considered statistically significant at p<0.05.

Patients at our Center at time of initial consultation sign a universal informed consent, which allows for the use of their medical records for clinical research as long as the patients' anonymity and the confidentiality of the medical record is maintained. Based on this informed consent, expedited review by the Center's IRB Chair suffices for a study like the one here reported. A confirmatory letter from the Chairman of the Center's IRB is available on request. All patients undergoing FMR1 testing in addition sign a test-specific informed consent prior to blood draw.

### Results of Example 4

Table 4 defines the study population by primary infertility diagnoses [as listed on the medical record and mandated by the Centers for Disease Control (CDC) in annual assisted reproduction outcome reports] and race/ethnicity (as self reported).

TABLE 4
Infertility diagnoses per race/ethnicities¹

| | | | |
|---|---|---|---|
| CAUCASIAN | AFRICAN | ASIAN | Total |

INFERTILITY PATIENTS:
n = 344

| | | | | | | | |
|---|---|---|---|---|---|---|---|
| MALE FACTOR | 36 | (15.6) | 7 | (16.3) | 16 | (22.9) | 59 |
| ENDO- | 8 | (3.5) | 3 | (7.0) | 2 | (2.9) | 13 |
| METRIOSIS | | | | | | | |
| POLYCYSTIC | 15 | (6.5) | 2 | (4.7) | 2 | (2.9) | 19 |
| OVARIES | | | | | | | |
| DIM. OV. | 114 | (49.4) | 23 | (53.5) | 29 | (41.4) | 166 |
| RESERVE | | | | | | | |
| TUBAL | 29 | (29.6) | 10 | (23.3) | 14 | (20.0) | 53 |
| INFERTILITY | | | | | | | |
| UTERINE | 5 | (2.2) | 5 | (11.6)³ | 0 | (0.0) | 10 |
| UNEXPLAINED | 3 | (1.3) | 1 | (2.3) | 1 | (1.4) | 5 |
| OTHER² | 106 | (45.9) | 21 | (48.8) | 35 | (50.0) | 162 |
| TOTAL | 316 | (136.8) | 72 | (167.4) | 99 | (141.4) | 487 |

OOCYTE DONORS:
n = 41

| | | | | | | | |
|---|---|---|---|---|---|---|---|
| NORMAL | 23 | (92.0) | 6 | (100.0) | 9 | (90.0) | 38 |
| FINDINGS | | | | | | | |
| DIM. OV. | 2 | (8.0) | 0 | (0.0) | 1 | (10.0) | 3 |
| RESERVE | | | | | | | |
| TOTAL | 25 | (100.0) | 6 | (100.0) | 10 | (100.0) | 42 |
| TOTAL STUDY | 341 | | 78 | | 109 | | 528 |
| GROUP | | | | | | | |
| n = 385 | | | | | | | |

¹Some patients had more than one primary infertility diagnosis;
²Represents a multitude of other diagnoses, including recurrent miscarriages, desire for preimplantation genetic diagnosis, same gender couples and single women.
³The only statistically significant difference in this table was noted in the higher prevalence of uterine disease in African than Caucasian women (p < 0.05).

As shown in Table 4, three-hundred forty four (89.4%), out of 385 study subjects, were infertility patients. The remaining 44 (10.6%) were prescreened oocyte donors, with presumed normal ovarian function, though 3 were later diagnosed with diminished ovarian reserve (Table 4).

Average age for the whole study population was 35.5±6.7 years (egg donors, 24.1±3.7; infertility patients, 36.8±5.3 years; p<0.001).

The most frequent infertility diagnosis, representing 166 out of a total 487 diagnoses (34.1%), was diminished ovarian reserve, followed by “other” (162/487; 33.3%). The most infrequently encountered diagnosis was so-called unexplained infertility in 5/487 (1.0%) of patients. Based on age-specific ovarian function testing (Barad et al., 2007), three oocyte donors (7.3%) were also diagnosed with diminished ovarian reserve.

A high prevalence of diminished ovarian reserve in this study population is also supported by the observation that, amongst women with known anti-Müllerian hormone (AMH) levels, 56% demonstrated levels below 0.8 ng/ml, reflecting diminished ovarian reserve at al ages (Brad et al., 2007). As expected, only two (later disqualified) donors (4.8%) demonstrated such low AMH levels. Race/ethnicity-specific ages and AMH levels are presented in Table 5.

TABLE 5
Patient characteristics

| | | |
|---|---|---|
| CAUCASIAN | AFRICAN | ASIAN |

PATIENTS (n = 344)

| | | | |
|---|---|---|---|
| Age (years) | 37.2 ± 5.0 | 37.3 ± 5.8 | 36.4 ± 5.8 |
| AMH levels (ng/ml) | 1.6 ± 2.9 | 1.7 ± 2.7 | 1.3 ± 1.6 |
| % AMH < 0.8 ng/ml | 0.5 ± 0.5 | 0.5 ± 0.5 | 0.5 ± 0.5 |

OOCYTE DONORS (n = 41)

| | | | |
|---|---|---|---|
| Age (years) | 28.3 ± 3.5 | 23.8 ± 3.6 | 26.4 ± 3.6 |
| AMH levels (ng/ml) | 3.0 ± 2.1 | 3.1 ± 1.9 | 3.4 ± 2.0 |
| % AMH < 0.8 ng/ml | 0.1 ± 0.2 | 0.0 | 0.0 |

TOTAL STUDY GROUP (n = 385)

| | | | |
|---|---|---|---|
| Age (years) | 35.7 ± 6.4 | 35.5 ± 7.2 | 35.0 ± 6.5 |
| AMH levels (ng/ml) | 1.7 ± 2.8 | 1.9 ± 2.7 | 1.6 ± 1.8 |
| % AMH < 0.8 ng/ml | 0.5 ± 0.5 | 0.4 ± 0.5 | 0.4 ± 0.5 |

No significant differences were noted between races/ethnicities in any of the parameters listed in the table. Patients and oocyte donors differed in all races in all three parameters (p < 0.001).

As the table demonstrates, there were no differences between the three racial groups.

Caucasians represented a large majority amongst infertility patients (229/344; 66.6%) and donors (25/41; 61.0%), for a combined 65.2% (254/385), followed by Asians (81/385, 21.0%) and African (50/385, 13.0%) (Table 3).

Table 6 also summarizes the normal and abnormal CGG repeat numbers amongst races/ethnicities in the four study groups.

TABLE 6
Triple CGG count distribution amongst racial/ethnic groups

| | CGG COUNTS | | | | |
|---|---|---|---|---|---|
| | GROUP 1 | GROUP 2 | GROUP 3 | GROUP 4 | |
| Allele-1 | Normal | Abnormal low | Normal | Abnormal low | |
| Allele-2 | Normal | Normal | Abnormal high | Abnormal high | TOTAL |
| CAUCASIAN | 138 (54.3) | 68 (26.8) | 42 (16.5) | 6 (2.4) | 254 (71.0) |
| n (%) | | | | | |
| AFRICAN | 32 (64.0)¹ | 13 (26.0) | 5 (10.0) | 0 | 50 (14.0) |
| n (%) | | | | | |
| ASIAN | 54 (66.7)¹ | 9 (11.1) | 18 (22.2) | 0 | 81 (22.6) |
| n (%) | | | | | |
| TOTAL | 224 (58.2) | 90 (23.4) | 65 (16.9) | 6 (1.6) | 385 (100.0) |
| n (%) | | | | | |

¹African and Asian women demonstrate as significantly higher prevalence of two normal allele counts than Caucasians (p < 0.03)

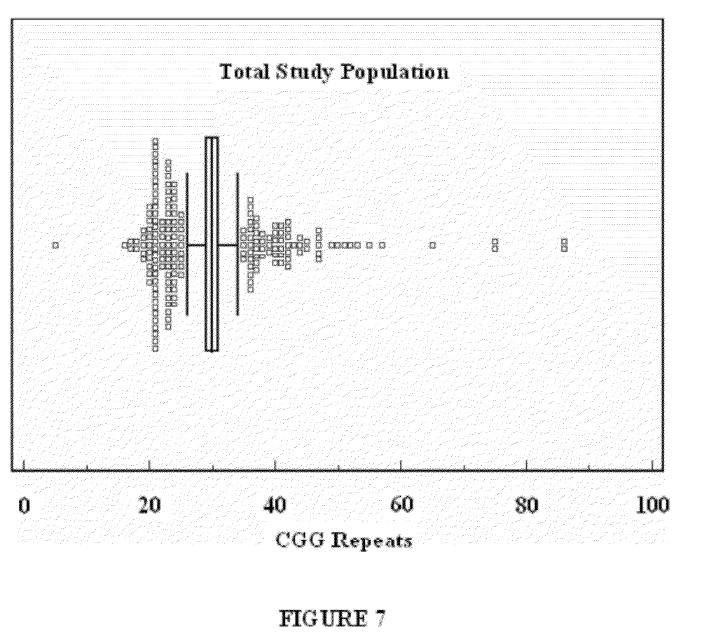

FIG. 7 depicts the box and whisker plot for triple CGG counts on the FMR1 gene for the whole study population. We have found that the normal range of triple CGG nucleotides on the FMR1 gene for ovarian function is 26 to 34 repeats. As the figure demonstrates, the median count, involving 770 alleles, was 30 repeats, corresponding to the distribution spike reported by Fu et al between 29 and 30 repeats (Fu et al., 1991). The 25^{th} to 75^{th} percentile of normal distribution, representing 50% of all cases with an interquartile range of two repeats, is defined by 29 to 31 CGG repeats (see box in FIG. 7). An additional 8% of women fell into the area between the two vertical bars, defined by 26 and 34, CGG repeats, respectively. The normal range of triple repeat counts for the patient population in this study was, therefore, defined as 26 to 34 repeats (see also Materials and Methods) and a total of 224/385 (58.2%) of all patients demonstrated two normal allele counts within that range (Table 6).

Another 155 women (43.3%) demonstrated one allele outside of this normal range and only 6 (1.6%) showed both alleles outside of normal range.

In comparing the prevalence of abnormal triple CGG counts amongst the three racial/ethnic groups, African and Asian women had a higher prevalence of two normal count alleles (65.6%) than Caucasians (54.3%; Chi Square 4.6; df=1; p=0.03).

Caucasians had also the highest rate of one allele abnormalities (43.3%), followed by Africans (37.0%) and Asians (33.3%) and also were the only patients who demonstrated a prevalence (2.4%) of two allele abnormalities (Table 6).

The three racial/ethnic groups also demonstrated significant prevalence differences in outliers. Specifically, Asians were significantly less likely to have abnormally low triple CGG counts (≦25) in comparison to Caucasians (p=0.002) and African women (p=0.03). The data fail to demonstrate statistically significant racial/ethnic differences in the high abnormal range (≧33 repeats). Relatively small patient numbers, however, do not preclude a type II error since trends towards fewer (amongst African) and more (amongst Asian) high count women were apparent (Table 6).

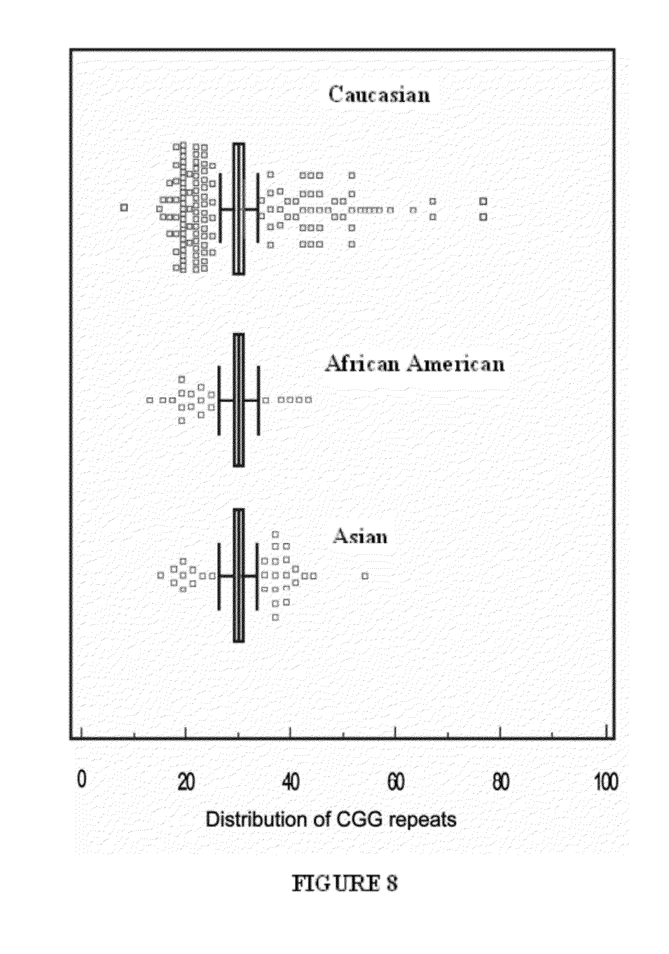

FIG. 8 presents a compendium of individual box and whisker plots for the three racial/ethnic study groups. It is worthwhile to note that all three racial/ethnic groups demonstrated identical median counts and normal count ranges.

### Discussion of Example 4

Fu et al initially reported the distribution pattern for triple CGG repeats in a general population (Fu et al., 1991). We have been intrigued by the fact that, based on these data, a majority of alleles in the general population demonstrate between 26 and 34 triple CGG repeats. This narrow distribution peak for a majority of alleles suggested to us that there may be physiological significance to this range and, by implication, to outliers below and above this range (Gleicher et al., 2009a; Gleicher et al., 2009c).

Investigating, as had recently been suggested by an NIH working group [Fragile X-Primary Ovarian Insufficiency (F-POI) Working Group, 2008] whether the distribution of triple CGG repeats on the FMR1 gene varied between races/ethnicities, we in this study utilized a new statistical approach to assess what represents normal CGG counts (Tukey J., 1977). The resulting definitions of normal, though in contrast to Fu's general population (Fu et al., 1991) obtained in mostly infertile women, was, nevertheless remarkably similar.

The median triple CGG count for the whole study group was 30 and, thus, well within the distribution peak of 29 to 30, reported by Fu and associates. The hypothetically normal range, defined by box and whisker plots, was 26 to 34 repeats, —again similar to Fu's distribution peak and to normal cut off ranges for triple CGG counts, at which we previously reported increasing risk for premature ovarian senescence to begin (Gleicher et al., 2009a; Gleicher et al., 2009c).

The here presented data, therefore, not only strengthen the hypothesis that there is physiological meaning to the observed distribution peak for triple CGG repeats between 26 to 34, but, in addition, suggest that this distribution peak defines life-long normal ovarian function, while deviations from this range define increased risk towards premature ovarian senescence (Gleicher et al., 2009a; Gleicher et al., 2009c).

Such an interpretation is also supported by the fact that the median triple CGG repeat number of 30 is exactly where Chen et al reported the switching point between positive and negative translation effects of the gene product of the FMR1 gene (Chen et al., 2002). At least in animals, this gene product has, indeed, been implicated in germ cell proliferation in females and males (Bächner et al., 1993) and gene expression has been reported in the human fetal ovary (Rife et al., 2004).

The excellent correlation between here reported and previously published data also validates the utilized new statistical methodology to determine what should be considered normal triple CGG repeat numbers in reference to ovarian function. These numbers are further validated since, despite significant racial differences in triple CGG distribution, the range of normal CGG counts is exactly the same for patients of all races/ethnicities (FIG. 8).

This study demonstrates that there are a number of distinct differences in triple CGG count distributions amongst different races/ethnicities: Caucasian women to a statistically significant degree are less likely to demonstrate two alleles with normal CGG counts than either Asians or Africans. Caucasians in our patient population also were the only ones with two alleles with abnormal CGG counts (Table 6). They, thus, appear to demonstrate the most inhomogeneous distribution pattern, while Asians seem most homogenous (FIG. 8). This observation also complements previously reported observations that Caucasians, at a prevalence of approximately 1:246 (Crawford et al., 2001), probably demonstrate more premutation range CGG expansions than other ethnicities/races (Crawford et al., 2002).

Reverting to previously noted statistical associations between numbers of triple CGG repeats on the FMR1 gene and ovarian function (Gleicher et al., 2009a; Gleicher et al., 2009c), these observations also support previous reports, which suggested that ovarian function in Asian women differs from Caucasians. For example, Asian women experience poorer IVF outcomes than Caucasian patients (Purcell et al., 2007). In attempts to explain these findings, we reported that Chinese oocyte donors demonstrated an approximately 30-fold increase in risk towards premature ovarian senescence in comparison to Caucasian egg donors (Gleicher et al 2007). Such an obviously racial/ethnic difference in ovarian function should be of genetic nature.

Since abnormalities in triple CGG counts appear to represent the most frequent genetic cause of premature ovarian senescence (Wittenberger et al., 2007; Gleicher et al., 2009a; Gleicher et al., 2009b; Gleicher et al., 2009c), one would expect Asian patients to be disproportionally more frequently represented amongst either low or high outliers, both of which have been associated with increased risk towards premature ovarian senescence (Gleicher et al., 2009a; Gleicher et al., 2009c). Concomitantly, they should be underrepresented amongst women with normal CGG repeat counts. Amongst women with two normal allele counts (Group 1) Asian women were, however, quite definitely not underrepresented. Indeed, they demonstrated an above average prevalence in this group, though differences did not reach significance. Both, Asian and African women were, however, to a significant degree underrepresented amongst single allele low CGG counts (Group 2), while women with a single abnormally high allele count (Group 3), indeed, appear to demonstrate a compensatory higher prevalence amongst Asians. Probably because of relatively small patient numbers, this difference, however, also failed to reach significance (Table 6).

Absence of demonstrable differences in Asians in either abnormally low or high triple CGG counts suggests, in view of their reported increased risk towards premature ovarian senescence, that their risk may not be of genetic nature or may have an FMR1-independent, genetic association. Premature ovarian senescence is, for example, independently also associated with abnormal autoimmune function (Gleicher et al., 2009b). Asian women, therefore, may carry a higher risk towards such an autoimmune etiology.

Studying a female infertility population does not always allow extrapolation to general populations. This needs, as a matter of principle, to be restated. At the same time, the constancy of median triple repeat and normal range counts in different racial/ethnic groups, and the similarity of results with those reported by Fu et al for a general population (Fu et al., 1991), suggest that box and whisker plots, and determination of outliers by this statistical methodology, allows definition of more universal results, even if patients are not representative of general populations. Further studies of normal populations appear, however, still indicated in confirmation of the here reported findings.

In summary, racial/ethnic groups appear to differ significantly in triple CGG distributions on the FMR1 gene. Considering the evolving understanding about an association between abnormal CGG counts and risk towards premature ovarian senescence, it is tempting to speculate that these differences may, at least in part, be responsible for reported differences in ovarian function and fertility parameters amongst races/ethnicities.

### Example 5

Here, we defined a normal CGG repeat count on the FMR1 (fragile X) gene as between 26-34, and studied whether women with normal, heterozygous and homozygous alleles follow distinct ovarian aging curves.

In more detail, with regard to ovarian reserve, 26-34 triple CGG repeats on the FMR1 gene denote ‘normal’. This study explores whether two-allele analyses reflect risk towards diminished ovarian reserve based on age in consecutive patients (34 oocyte donors and 305 infertility patients), longitudinally and cross-sectionally. Box and whisker plots confirm the normal range of CGG counts. Patients were then defined as normal with both alleles in range, as heterozygous with one allele outside and homozygous with both alleles outside of range. Ovarian reserve was assessed by anti-Mullerian hormone (AMH). Normals at young ages exhibited significantly higher AMH concentrations than either heterozygous or homozygous females (p=0.009). By approximately age 35, heterozygous women have higher AMH concentrations than normal women, while homozygous women exceed normal women shortly before age 50 years. This data supports a control function of the FMR1 gene over ovarian reserve, thus defining life-long ovarian reserve patterns. Heterozygous and homozygous abnormal CGG counts reduce ovarian reserve at younger ages and improve ovarian reserve at older ages. They, thus, at expense of reduced fertility in the young, preserve fertility into older age. This function of potential evolutionary importance may explain the preservation of the FMR1 gene despite its, at times, serve neuropsychiatric risks.

### Materials and Methods of Example 5

Due to a high prevalence of prematurely diminished ovarian reserve, and recommendations for FMR1 testing in such women, as of January 2007, 469 consecutive patients had been tested at our center. Concomitantly, based on advantages over other ovarian reserve tests, such patients since mid-2007 also have been evaluated with anti-Müllerian hormone (AMH). Rohr et al recently confirmed that AMH is a better marker than follicle stimulating hormone (FSH) for early decline of ovarian reserve in women with longer FMR1 repeat alleles. Three-hundred-thirty-nine consecutive women with FMR1 and AMH evaluations, therefore, represent the study population.

A total of 339 consecutive women with FMR1 and AMH evaluations, represent the study population: thirty-four were anonymous egg donors, and 305 infertility patients, who had completed at least one in vitro fertilization (IVF) cycle. Since decline in ovarian reserve accelerates between ages 37 to 38, complicating the separation between premature and physiologic ovarian senescence, the study population was stratified for under, and above, age 38 years, with 153 infertile women under age 38 and 34 young egg donors considered a primary study population, largely unaffected by physiological ovarian aging.

At our center approximately 40 to 50 percent of young infertility patients under age 38 can, however, be expected to suffer from premature ovarian senescence, defined by baseline FSH levels that exceed the 95 percent confidence interval (95% CI) of an age-matched infertile patient population, and by significantly decreased oocytes yields.

Egg donors have to be under age 34 and undergo a multi-stage screening process, as mandated by US Food and Drug Administration guidelines. The donor's ovarian reserve and FMR1 assessments take place in a first round of medical testing. Donors, thus, are distinctively different from infertility patients, with donors on average younger and exhibiting better ovarian reserve parameters (Table 1). They, therefore, can be considered functional controls for the center's infertility patients, most closely resembling a normal starting point for ovarian reserve after menarche. Patient groups are, therefore, independently presented, unless otherwise indicated.

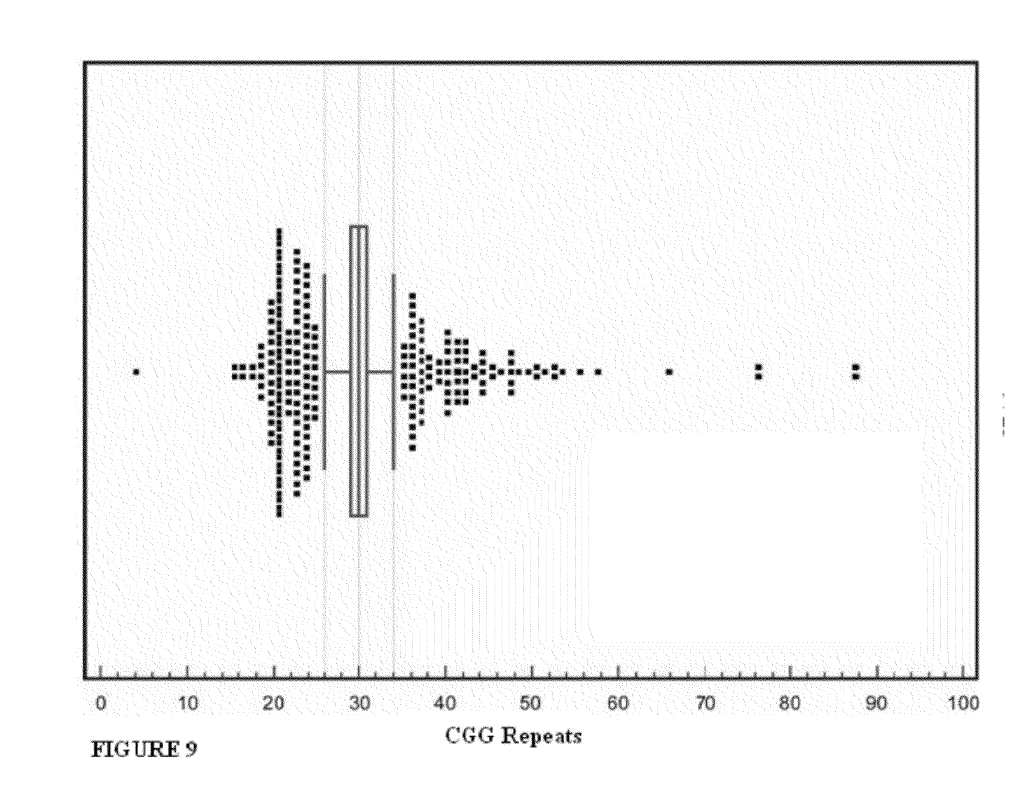

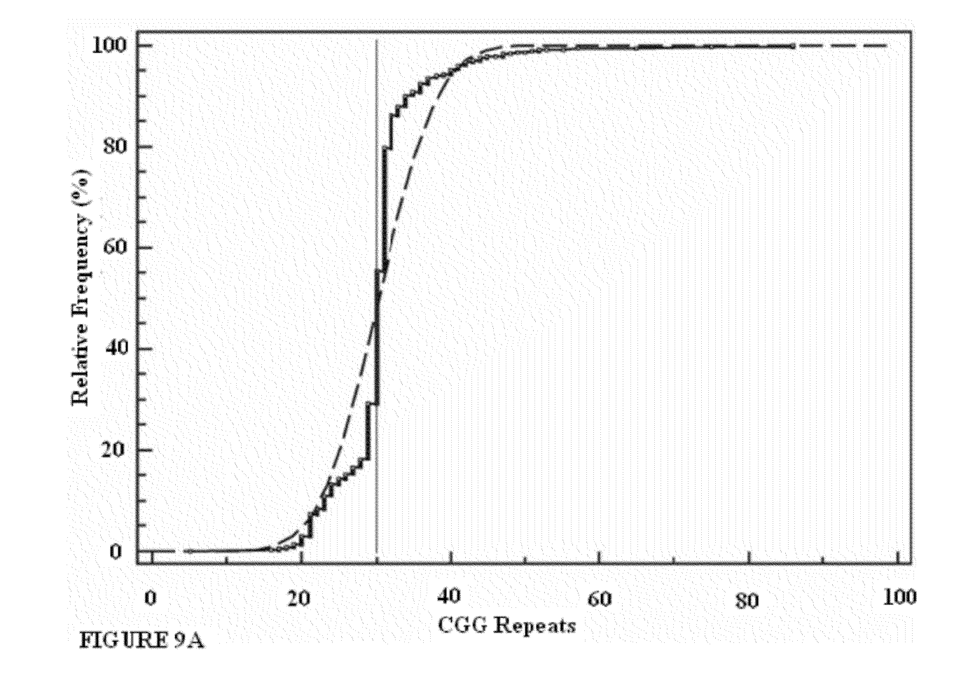

Using CGG repeat numbers of the entire study population (FIG. 9, see also FIG. 9A) a normal CGG triple count, in respect to ovarian function, was, based on box and whisker plots, reconfirmed at 26 to 34 repeats (median 30). Box and whisker plots are configured by establishing a “box,” which contains the middle 25^{th} to 75^{th} percent percentile, representing 50 percent of all cases. Then, in addition, so-called “outside” values are established, defined as smaller than the lower quartile minus 1.5-times the so-called interquartile range, or larger than the upper quartile, plus 1.5-times the interquartile range.

This normal range appears further validated by its congruity with the normal range, previously defined in another study of different races/ethnicities, its correspondence to the large distribution peak of CGG repeats in general populations, reported by Fu et al between 29 and 30 repeats and the fact that the here once again confirmed median range of normal at 30 CGG repeats has been reported by Chen et al to represent the switching point from positive to negative effects of the FMR1 gene product.

Women were, thus, considered normal in CGG counts if both of their FMR1 alleles demonstrated between 26 and 34 alleles. They were considered heterozygous if one allele was in the normal range of 26 to 34 and the other allele was either above 34 (≧35) or below 26 (≦25), both ranges associated with increased risk towards premature ovarian senescence and considered homozygous if both alleles demonstrated either ≧35 and/or ≦25 CGG counts.

Since it has previously demonstrated that AMH, but not follicle stimulating hormone (FSH), statistically correlates with triple CGG counts below the premutation range, and Rohr et al demonstrated the same for the premutation range, AMH was utilized to assess ovarian reserve. AMH is the more accurate reflection of ovarian reserve as long as ovarian function is relatively normal; i.e., under age 38 and before, but not after chemotherapy. FSH levels and oocytes yield were, as additional reflections of ovarian reserve, assessed in parallel where indicated.

FMR1 gene analyses and AMH testing were, as previously reported, performed on blood samples by commercial assays (Genzyme Analytical Services, Westborough, Mass., Quest Diagnostics, Lyndhurst, N.J. and LabCorp, Burlington N.C.). In brief, FMR1 assays were performed by testing DNA by Southern blot and polymerase chain reaction (PCR) to determine the size and methylation status of the CGG repeats. Southern blot analysis was performed with the probe St.B12.3 on EcoR1 and Eagle digested DNA. PCR products were generated using fluorescent labeled primer and sized by capillary electrophoresis. AMH testing was performed using an enzyme-linked immunoabsorbent assay (ELISA) (MIS/AMH ELISA DSL-10-14400). FSH and estradiol assays were, as also previously reported, run in house, utilizing a standard ELISA assay (AIA-600 II, Tohso, Tokyo, Japan). Only results in assay range were considered for statistical evaluation. Where patients had more than one test on their medical record, only the initial test was utilized.

Continuous variables were tested for normality with the Kolmogorov-Smirnov test. Normally distributed variables are shown as means±standard deviation (SD), while variables, corrected for normality by log conversion, are presented as geometric means and 95 percent confidence interval (95% CI) of the mean. Rates are presented as raw numbers and percentages.

All data analyses were performed using SPSS for windows, version 15.0. Demographic and biochemical data were assessed by analysis of variance, Chi square or Fisher's exact test. Differences were considered statistically significant at p<0.05.

Every patient at our center signs at time of initial registration an informed consent, which allows for review of medical records for research purposes, as long as such a review does not impair confidentiality of the medical record and protects anonymity of patients. If these conditions are met, expedited IRB review suffices. This study was IRB approved. A confirmatory letter from the Chairman of the center's IRB is available upon request. Our center also follows suggested guidelines in reference to genetic testing. Patients undergoing FMR1 testing, therefore, signed a test-specific informed consent before blood draw.

### Results of Example 5

Table 7 summarizes patient characteristics for egg donors and infertility patients separately and for the combined younger patient population, made up of egg donors and infertility patients under age 38.

TABLE 7
Patient characteristics*

| | | | | |
|---|---|---|---|---|
| Age | AMH | FSH | Estradiol | Oocytes |
| (years) | (ng/ml) | (mIU/ml) | (pg/ml) | (n) |

EGG DONORS (n = 34)

| | | | | | |
|---|---|---|---|---|---|
| Normal CGG count | 25.7 (23.5-27.9) | 2.9 (2.1-3.7) | 5.0 (−17.3-27.2) | 25.0 (25.0-25.0) | 21.8 (10.2-33.2) |
| Heterozygous | 26.1 (24.5-27-8) | 3.5 (2.2-4.8) | 4.7 (3.9-5.5) | 37.3 (−34.4-109.1) | 13.8 (5.3-22.3) |
| Homozygous | 23.0 (16.7-29.3) | 3.4 (−3.0-9.8) | — | — | 13.5(−43.6-70.7) |
| TOTAL | 25.7 (24.5-27.0) | 2.7 (2.1-3.4) | 7.9 (2.8-22.8) | 27.5 (9.4-80.4) | 16.6 (11.7-21.6) |

INFERTILITY PATIENTS < AGE 38
(n = 153)

| | | | | | |
|---|---|---|---|---|---|
| Normal | 33.4 (32.6-34.2) | 1.3 (1.0-1.8)¹ | 9.5 (8.2-11.2) | 46.7 (41.1-52.4) | 9.4 (7.4-11.8)¹ |
| Heterozygous | 33.9 (33.1-34.6) | 1.1 (0.8-1.4) | 10.6 (8.7-12.8) | 43.2 (37.9-49.1) | 6.7 (4.4-9.0)¹ |
| Homozygous | 34.4 (32.9-35.8) | 0.6 (0.3-1.0)¹ | 13.2 (8.6-20.1) | 50.4 (35.8-65.0) | 6.0 (1.9-10.1) |
| TOTAL | 33.7 (33.2-34.2) | 1.1 (0.9-1.3) | 10.3 (9.2-11.6) | 42.5 (39.0-46.3) | 8.9 (7.3-10.4) |

≧ AGE 38
(n = 152)

| | | | | | |
|---|---|---|---|---|---|
| Normal | 42.6 (42.0-43.1) | 0.4 (0.3-0.5) | 10.8 (9.0-12.9) | 48.2 (42.3-55.0) | 4.1(2.6-5.6) |
| Heterozygous | 42.5 (41.8-43.1) | 0.5 (0.4-0.7) | 10.2 (8.6-12.0) | 50.8 (44.6-57.8) | 7.3 (4.5-10.1) |
| Homozygous | 41.6 (40.5-42.8) | 0.9 (0.0-1.8) | 23.8 (8.9-38.8) | 52.9 (19.1-86-8) | 3.0 (0.5-5.5) |
| TOTAL | 42.4 (42.1-42.8) | 0.5 (0.4-0.6) | 10.8 (9.6-12.2) | 49.0 (44.7-53.7) | 5.6 (4.1-7.1) |

TOTAL YOUNG STUDY POPULATION
(EGG DONORS + INFERTILITY PATIENTS < AGE 38)
(n = 187)

| | | | | | |
|---|---|---|---|---|---|
| Normal | 32.1 (31.1-33.0) | 1.5 (1.1-1.9)¹ | 9.3 (8.0-10.9) | 46.0 (40.5-51.6) | 10.3 (8.2-2.9)¹ |
| Heterozygous | 32.1 (31.1-33.1) | 1.3 (1.0-1.7)¹ | 10.0 (8.3-12.1) | 46.3 (40.1-52.4) | 8.1 (5.7-10.5)¹ |
| Homozygous | 33.3 (31.4-35.3) | 0.7 (0.4-1.2)¹ | 13.2 (8.6-20.1) | 50.4 (35.8-65.0) | 7.3 (2.6-12.0)¹ |
| TOTAL | 32.2 (31.6-32.9) | 1.3 (1.1-1.5) | 10.0 (8.9-11.3) | 46.7 (42.8-50.7) | 10.1 (8.5-11.7) |

*FSH and estradiol levels were not available for all patients. Since not all egg donors have undergone an IVF cycle yet, retrieved oocytes numbers in egg donors also reflect only an incomplete sample size. All values are presented as means with 95% confidence interval in parenthesis.

As the table demonstrates, patients, under and above age 38 years were as expected significantly older than donors (p<0.001). They, however, demonstrated only a trend towards higher AMH levels (p=0.071). The table also demonstrates that amongst egg donors neither AMH nor FSH levels differed significantly between normal, heterozygous or homozygous, abnormal females. Patients under age 38 demonstrates, however, a significant trend towards lower AMH levels from normal, over heterozygous to homozygous (p=0.032) and in oocytes yield (p=0.018), but not in FSH or estradiol levels. AMH levels were significantly different between normal and homozygous women (p=0.009) but only demonstrated a trend between normal and heterozygous females (p=0.063). The significance was carried over when donors and women under age 38 were combined (AMH, p=0.027; oocytes yield, p=0.024) but disappeared for all ovarian function parameters in patients at, and above, age 38 years (all Table 7).

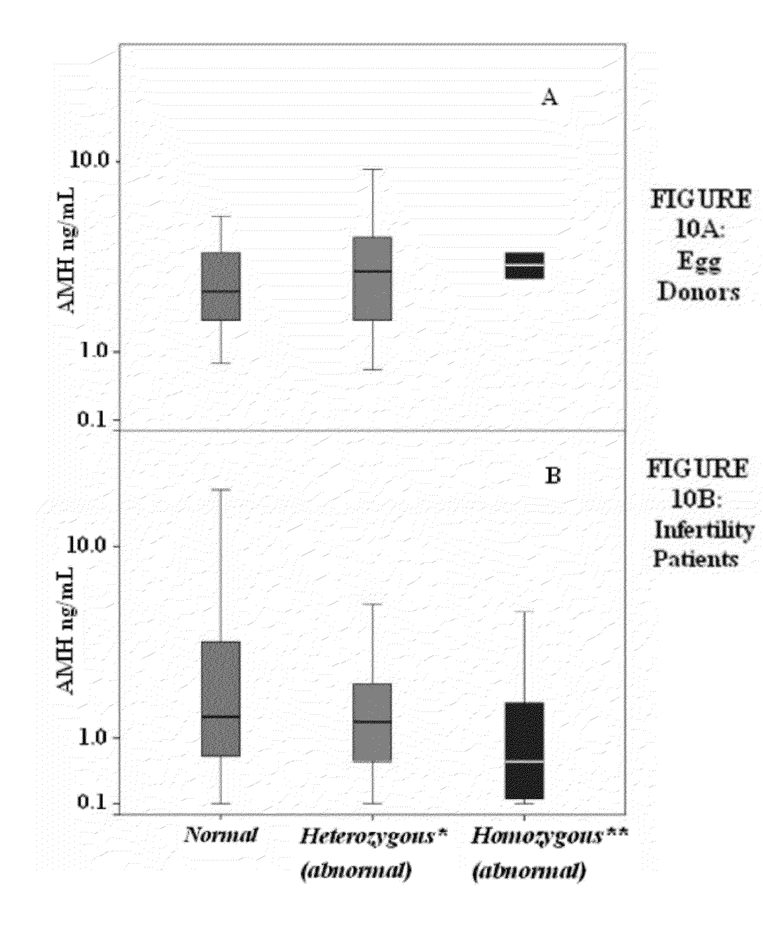

The effects of CGG triple repeat numbers in reference to the ovarian reserve parameter AMH is, however, best viewed graphically: FIGS. 10A and 10B demonstrate in the format of box and whisker plots in an upper panel, FIG. 10A, AMH levels in egg donors and in a lower panel, FIG. 10B, in infertility patients. As can be seen, panel A demonstrates that in egg donors AMH levels are very similar between normal, heterozygous and homozygous women. Indeed, if anything, a mild trend towards higher AMH levels from normal over heterozygous to homozygous, with concomitant narrowing of the range of counts, is apparent. AMH levels in young infertile women decrease to a significant degree from normal to heterozygous and homozygous (p=0.032). The difference between normal and homozygous women is significant (p=0.009), while the difference between normal and heterozygous misses significance but suggests a trend (p=0.063).

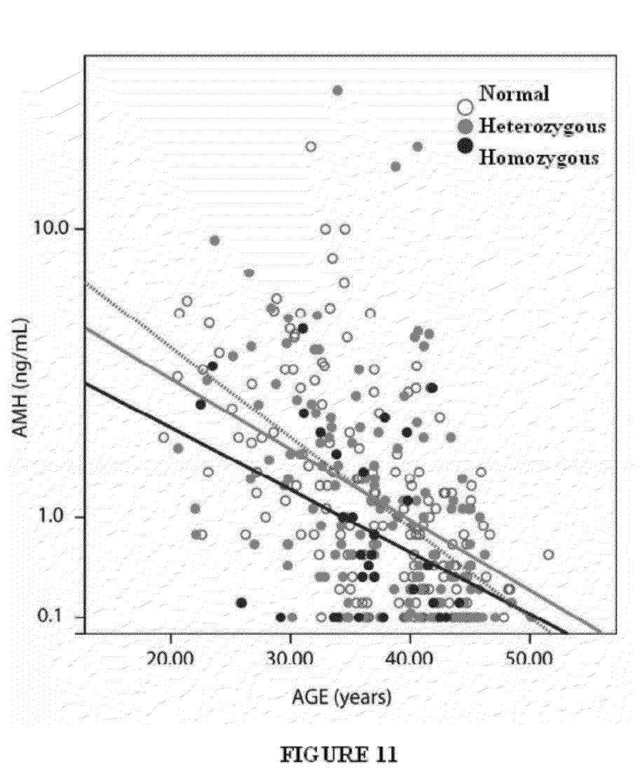

To better understand the effects of age on AMH levels, stratified for zygosity, FIG. 11 demonstrates a linear regression analysis of AMH levels, involving the total study population of all ages. As the figure demonstrates, linear correlations between normal and heterozygous women cross at approximately 35 years of age, with normal women at younger ages, and heterozygous women above approximately age 35, demonstrating higher AMH levels. Homozygous women, in contrast, almost throughout, demonstrate the lowest AMH levels, with the homozygous line crossing the normal patient line only close to age 50, and never crossing the heterozygous line.

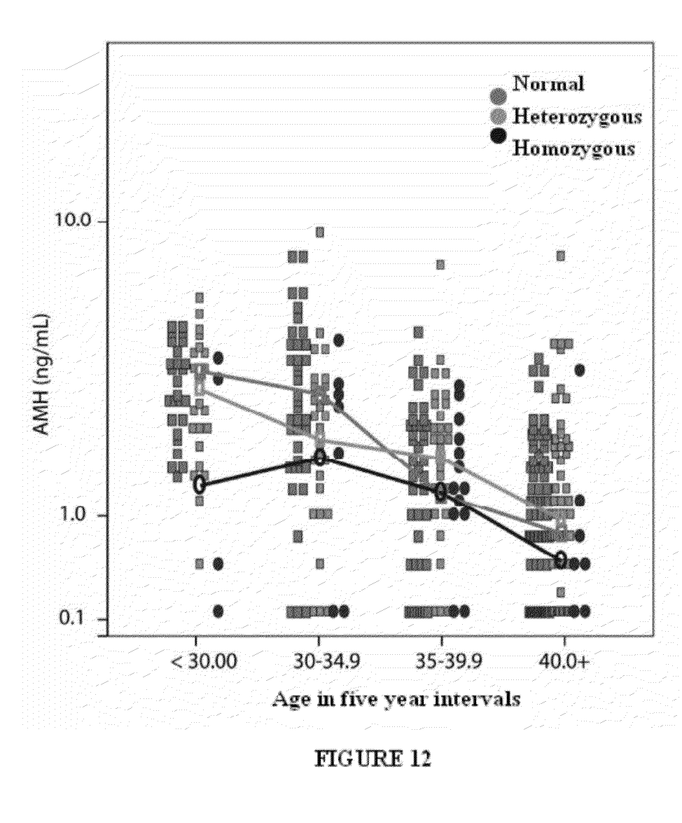

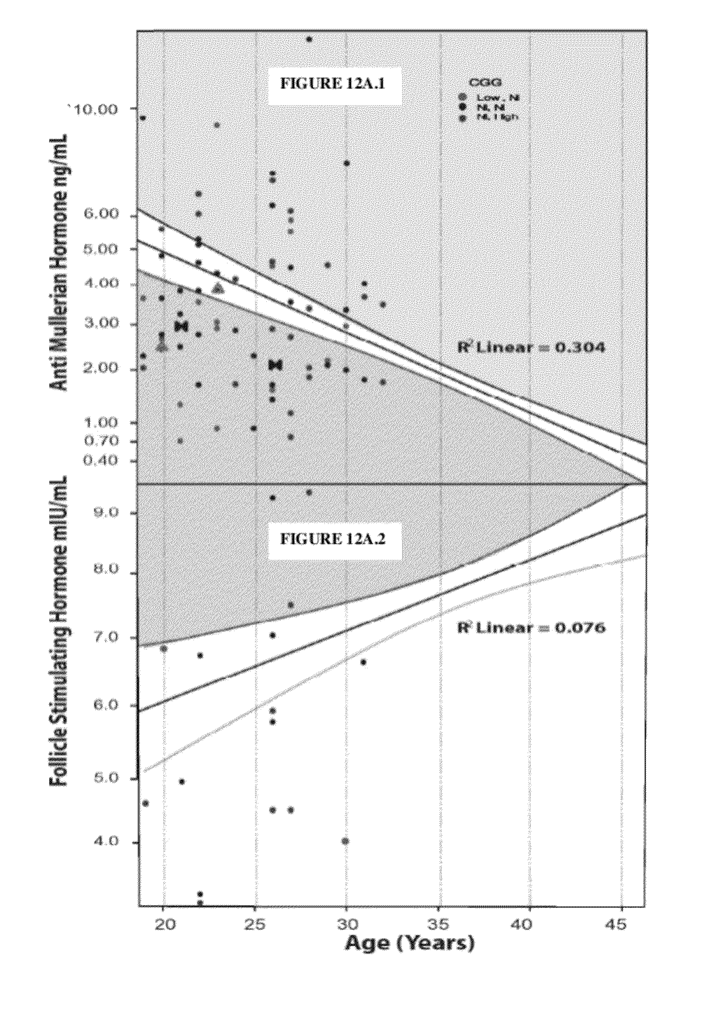

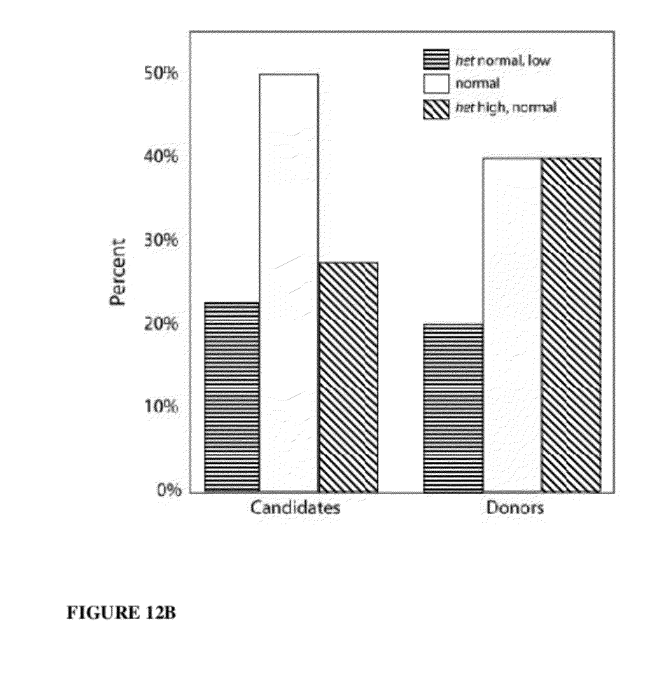

FIG. 12 demonstrates a binned age-stratified analysis of AMH levels in four age intervals, again combining egg donors and infertility patients of all ages. This figure allows for further clarifications: As it demonstrates, AMH levels up to age 30 (<30 years) differ significantly between normal and homozygous women (p=0.032) but fail to do so between normal and heterozygous females (p=0.72). The difference between heterozygous and homozygous women also failed to reach significance, but is suggestive of a trend (p=0.09). Not included in the figure are data demonstrating in this age group significant differences in FSH levels between normal and homozygous women (p<0.001) and between heterozygous and homozygous females (p=0.002) (data not shown).

Overall, up to age 30 years, AMH (p=0.042), FSH (p<0.001) and oocytes yield in IVF (p=0.009) differed to statistically significant degrees between the three groups (data not shown). All statistically significant differences disappeared, however, above age 30 years.

As FIG. 12 demonstrates, normal patients demonstrated only a very mild decline in AMH levels up to age 35. Between age 35 and 40, their AMH levels, however, declined rapidly, only to return to a slower decline by age 40. This pattern contrasts from that of heterozygous and homozygous women, with the former demonstrating a slow, steady decline at all ages and the latter demonstrating the most interesting pattern: Homozygous women demonstrate very low AMH levels at youngest ages (<30 years), then somewhat increase their AMH until age 35 years, at what time they return to a, more or less, parallel pattern with heterozygous women, characterized by a slow, steady decline.

After age 40 years, all three groups follow, more or less, parallel patterns of AMH declines, with homozygous women apparently at all ages demonstrating the lowest AMH levels. The AMH curve of heterozygous females, however, after approximately age 33, crosses the curve of normal women, and maintains after that point higher AMH levels throughout.

### Discussion of Example 5

Since ovarian function is age dependent, this study offers the advantage of a young and presumably healthy control population (egg donors) separately, and in combination, with an infertility population (patients) of all ages. This is important because the here presented egg donors should to a large degree reflect ovarian reserve before it diminishes, either due to physiological aging or prematurely, as a consequence of abnormal CGG triple repeat counts on the FMR1 gene. Since this study involves both populations and since our center's patient population especially at young ages is to a significant degree affected by premature ovarian senescence, it allows, with a statistically robust patient sample of 339 women (678 alleles), for the longitudinal observation of ovarian reserve dynamics, based on zygosity of triple CGG counts on the FMR1 gene.

This study confirms prior data in support of strong statistical associations between numbers of CGG repeats on the FMR1 gene and ovarian reserve, but does so for the first time based on a defined normal range (26 to 34 triple CGG repeats), and depending on whether abnormal counts occur on only one (heterozygous) or two (homozygous) FMR1 alleles.

It had previously been suggested that the in the general population observed distribution peak of 29 to 30 CGG repeats, initially reported by Fu et al, may not be coincidental and may denote a normal range in a statistical and physiologic association between number of triple CGG repeats on the FMR1 gene and ovarian reserve. Abnormalities in CGG counts on both sides of this distribution peak seem to denote statistical risk towards premature ovarian senescence, with lower counts representing mildly more risk than higher counts.

This observation allowed us to take the next step and define formally normal CGG repeat counts via box and whisker plots as between 26 and 34 repeats (median 30). The definition of a normal range, in turn, allowed us to define outliers as abnormal, which for the first time permitted a statistical comparison of allele distribution counts in different races/ethnicities.

All of our prior studies, like Fu's initial work, were, however only based on single FMR1 allele counts. A confirmation of statistical associations between triple CGG counts on the FMR1 gene and ovarian reserve requires, however, also that such an association be dependent on heterozygous or homozygous expression of the genetic abnormality and the here presented study was, therefore, designed to investigate this point.

Assuming genetic control function of the FMR1 gene over ovarian reserve, young egg donors still largely unaffected by any etiology for DOR do not yet demonstrate a difference in ovarian reserve based on CGG counts (zygosity) (FIG. 10A). Only slightly older women (in the infertile population), however, already do. As one would expect for a genetic control function, very significant differences in ovarian reserve, based on CGG counts (zygosity) become apparent, with best ovarian reserve in normal, intermediate reserve in heterozygous and worst in homozygous women (FIG. 10B).

Utilizing AMH levels as the most reliable indicators of ovarian reserve at normal to premutation repeat CGG count ranges, FIG. 10A demonstrates that AMH levels in young egg donors are similar, whether they are normal, heterozygous or homozygous for CGG count abnormalities. Considering that very young women, such as egg donors, even if their ovarian reserve is prematurely impaired, will still have enough follicular activity to produce adequate AMH levels, this should not surprise. Somewhat older, infertile patients, however, already demonstrate significant negative effects of zygosity: While heterozygous infertile women still demonstrate similar AMH levels to normal females, homozygous women exhibit significantly reduced levels (FIG. 10B).

FIG. 11 further clarifies these findings in linear regression analyses. As the figure, combining egg donors and infertile women of all ages, demonstrates, young women exhibit a clear hierarchy of AMH levels, with normal females showing the highest and homozygous women the lowest. This pattern is, however, not maintained as women age: By approximately age 35 years, heterozygous women start exhibiting higher AMH levels than normal females and this difference in favor of heterozygosity increases further with advancing age. Homozygous females demonstrate higher AMH levels than normal women only close to age 50 (i.e., close to menopause), maintain an almost parallel line with heterozygous women at advanced female ages and never catch up in AMH levels.

An age-stratified analysis in five-year blocks (FIG. 12) further confirms this pattern but allows for some additional analysis: Young homozygous women, under age 30 years, clearly demonstrate the lowest AMH levels. Since AMH is reflective of small follicles, this suggests that at these very young ages homozygosity in CGG count abnormalities on the FMR1 gene is associated with either smaller inherent follicle numbers, or poorer recruitment from the dormant follicle pool. That homozygous women appear to slightly increase AMH at young ages (FIG. 12) may suggest that observed findings are not due to an inherently smaller follicle pool but to lower recruitment, which, in turn, can be viewed as an effort to preserve the follicular pool into older age.

After age 35, normal women demonstrate a rather rapid decline in AMH production until age 40, while heterozygous and homozygous females, originally starting from lower AMH levels, decline more modestly, resulting in the already in FIG. 11 noted crossover of the normal and heterozygous regression lines at, or just before, age 35 years. These distinctive patterns suggest differences in follicular recruitment between normal and heterozygous, as well as homozygous, women, with the latter two declining slower because of, possibly, smaller recruitment, resulting in lower AMH levels, though better follicular preservation.

From this point on, heterozygous women produce higher AMH levels than normal women, and even homozygous women closely approach AMH production of normal females. AMH production, thus, as widely reported, correlates with female age, but does so differently, depending on zygosity of triple CGG counts on the FMR1 gene.

These data confirm the expected hierarchical association between normal, heterozygous and homozygous, abnormal triple CGG counts on the FMR1 gene. As especially FIG. 11 well demonstrates, while abnormalities in triple CGG counts at early ages are associated with low AMH levels, they appear associated with improved, and especially prolonged, AMH production at later ages. This would suggest that the gene's function may lie in preserving, in subgroups of women, ovarian function into older age and would explain why, despite the obvious short-term disadvantages to female fertility and severe neuro/psychiatric disorders, especially in male offspring, abnormalities in CGG counts are so highly preserved.

These data also suggest that preservation of ovarian reserve into older age in heterozygous and homozygous patients very likely occurs via diminished recruitment. FIG. 12 demonstrates this best by showing that homozygous patients at very young ages apparently recruit the fewest follicles and, therefore, exhibit the lowest AMH levels. Normal women recruit the most and, therefore, demonstrate the highest AMH levels, with heterozygous women, as one would expect, in the middle range. Rapid recruitment may deplete availability of follicles and, therefore, normal women demonstrate between ages 35 and 40 apparently the fastest decline in ovarian reserve, reflected in the most severe drop in AMH levels amongst all three groups. By approximately age 40, ovarian reserve of all three genetic patterns has become similar, though, even at very advanced female ages, above 40 years, homozygous patients remain, almost until menopause, those with the lowest AMH levels (FIG. 11).

These findings are potentially contradictory to some widely held believes. For example, women with diminished ovarian reserve at young ages are expected to experience early menopause. Here presented data, in contrast, suggest that they may experience late menopause. While this remains to be determined, it is entirely conceivable that age at menopause represents yet another phenotypical differentiatiion between premature ovarian senescence due to FMR1 and autoimmune abnormalities.

These findings also suggest that follicular senescence data, reported by Faddy and co-workers, may require further differentiation: Accelerated decline in ovarian reserve, by Faddy attributed to approximately age 37.5 years, and a remaining pool of approximately 25,000 follicles, seems to occur only in normal women and only at younger ages (FIG. 12). The relative slow decline in ovarian reserve with heterozygous and homozygous CGG counts explains why Faddy et al, presumably in a population containing normal, heterozygous and homozygous patients, on average calculated a slightly later age for accelerated decline in ovarian reserve.

Finally, since abnormalities in CGG counts on the FMR1 gene appear to prolong ovarian function, it is tempting to speculate that affected women may be better candidates for fertility treatments and/or may be better responders to ovarian stimulation at advanced ages. Studies to address these and other questions are currently underway.

### Example 6

### Objective of Example 6

Donor eligibility screening traditionally included the search for diminished ovarian reserve (DOR) by follicle stimulating hormone (FSH) and attempts at eliminating hyperstimulation risks. More recently, other ovarian reserve parameters are increasingly utilized, raising the question whether they potentially improve donor selection.

### Materials and Methods of Example 6

consecutive women (age 19-33; mean 24.2±3.6) presented as potential egg donors. In absence of gross pathology on ultrasound, candidates underwent AMH and genetic testing (including FMR1; normal CGG count range 26-34). Abnormally low AMH (DOR) was determined based on age-specific levels from <0.5 ng/mL (age ≧41) to <2.1 ng/mL (age <30). AMH ≧4.5 ng/mL was considered suspicious of polycystic ovaries (PCO).

### Results of Example 6

AMH levels ranged from 0.7-13.0 ng/mL (mean 3.7±2.3). 14 (18.4%) women showed low AMH (DOR) and 19 (25.0%) women showed AMH suspicious of PCO. Only 32 women had normal CGG counts on both alleles (42.1%); 37 (48.7%) were heterozygous-abnormal (1 abnormal high or low); 4 (5.3%) were homozygous-abnormal (abnormal counts on both alleles). Only 11 (14.5%) demonstrated normal AMH and CGG counts. So far only 12/76 (15.8%) were matched and reached retrieval.

### Conclusions of Example 6

By history and interviews, even carefully preselected oocyte donor candidates still demonstrate significant ovarian reserve abnormalities, either predisposing towards DOR or PCO (hyperstimulation) risks. Utilization of new markers of ovarian reserve, such as AMH and CGG repeats one FMR1, will likely improve donor selection, thus reducing risks towards disappointing oocyte yields and hyperstimulation.

### Example 6A

### Overview of Example 6A

Background Overview

Accurate assessments of ovarian reserve (OR) in egg donor candidates are crucial for maximal donor selection. This study assesses whether recently reported new methods of OR assessment by age-specific (as-), rather than non-as (nas-) hormones, follicle stimulating hormone (FSH) and anti-Müllerian hormone (AMH), and triple nucleotide (CGG) repeats on the FMR1 (fragile X) gene have the potential of improving egg donor selection.

Methods Overview

Seventy-three consecutive egg donor candidates (candidates), amongst those 21 who reached egg retrieval (donors), were prospectively investigated for as-FSH, as-AMH and number of CGG repeats. Abnormal findings were assessed in candidates and donors and oocyte yields in the latter were statistically associated with abnormal FSH and AMH (>/<95% CI of as-levels) and with normal/abnormal numbers of CGG repeats (normal range 26-34).

Results Overview

Amongst candidates mean as-AMH was 3.8+/−2.8 ng/mL (37.0% normal, 3.0+/−0.7 ng/mL; 26.6% low, 1.5+/−0.5 ng/mL; and 37.0% high, 5.8+/−2.2 ng/mL). AMH among donors was 4.2+/−1.7 ng/mL (33.3% normal, 14.3% low, and 52.4% high), yielding 17.8+/−7.2 oocytes, 42.9% in normal range (10-15), 9.5% in low (less than or equal to 9) and 47.6. % in high range (16-32). Candidates in 41.9% and donors in 38.1% demonstrated normal CGG counts; the remaining were mostly heterozygous abnormal.

Discussion Overview

Prospective assessment of even carefully prescreened candidates and donors still demonstrates shortcomings on both ends of the OR spectrum. Utilization of ovarian reserve testing methods, like as-hormones and CGG repeats on the FMR1 gene have potential of improving candidate selections.

Background of Example 6a

Oocyte donor selection in the United States (U.S.) represents a highly complex process, catering to different, guidelines and regulations. A dominant medical need, not affected by federal regulations, is to ascertain normal ovarian function. Prematurely diminished ovarian reserve (PDOR) in young donor candidates will negatively affect oocytes yields, while excessive ovarian response to stimulation can not only result in poor oocytes quality but lead to ovarian hyperstimulation syndrome (OHSS), and endanger the physical wellbeing of potential donors.

As for infertility patients, ovarian reserve (OR) in oocyte donor candidates is traditionally assessed via baseline follicle stimulating hormone (FSH), especially at younger ages reported to be a rather poor tool to detect PDOR. Anti-Müllerian hormone (AMH) appears to better reflect OR, and is, therefore, increasingly utilized to detect diminished ovarian reserve (DOR) and/or hyperstimulation risk in women with polycyctic ovarian syndrome (PCOS). No studies on AMH utilization in oocyte donor selection have, however, so far been performed. This study does this.

Assessments of OR in infertile women as well as oocytes donors, historically, practically exclusively, involve non-age-specific (nas-) levels of FSH and/or AMH. FSH, however, increases, and AMH decreases with advancing female age, which makes such non-discriminatory use of normal cut off values appear illogical. We, therefore, proposed utilization of age-specific (as-) cut off values for FSH and AMH, both found to be superior in determining OR: AMH at all ages and FSH under age 38 years discriminate better between higher and poorer oocytes yields with in vitro fertilization (IVF) than previously utilized nas-values. The utilization of as-AMH can also be helpful in determining ovarian hyperstimulation risk. How as-OR testing impacts oocyte donor selection has, however, so far, not been investigated. This study investigates this issue.

Numbers of CGG triple nucleotide repeats on the FMR1 gene statistically relate to risk towards PDOR, with 26 to 34 repeats representing a normal range of counts in regards to the genes effects on ovarian function (to be differentiated from normal and abnormal ranges of repeats in regards to neuro/psychiatric risks associated with the gene). Deviations from this normal range denote specific ovarian aging patterns, defined by varying rates of decline in OR. In an infertile population, CGG counts also correlate to OR, as reflected by oocytes yields in IVF. A specific heterozygous (normal/low) CGG repeat pattern appears to predispose towards a normal weight polycystic ovary phenotype.

The evaluation of triple CGG counts on the FMR1 gene, thus, also offers potentially important clinical information about OR in young women. What such evaluations could contribute to egg donor screening has, however, not previously been investigated, and represents, therefore, together with as-FSH and as-AMH, the third new tool evaluated in this study for its potential to improve oocytes donor selection.

### Materials and Methods of Example 6a

Patient Population

Since July, 2007, we have evaluated 164 applicants as potential egg donors who, based on a detailed questionnaire, qualified for further investigation. Amongst those, 73 (ages 19 to 33 years, mean 24.2+/−3.6) reached, after two interviews, a first laboratory screening stage, thus becoming candidates for egg donation. At this stage they underwent vaginal ultrasonography, random AMH and genetic testing, including determination of number of triple nucleotide (CGG) repeats on the FMR1 (fragile X) gene, for which patients signed an FMR1-specific informed consent.

Laboratory Assays

Since FSH, in contrast to AMH, requires timed blood draws, it was only evaluated in candidates who became donors by reaching ovarian stimulation.

FSH, estradiol and AMH were evaluated as previously described. In short, FSH and estradiol were obtained on cycle days 2/3 and assessed utilizing a standard enzyme-linked immunoabsorbent assay (ELISA, AIA-60011, Tosho, Tokyo, Japan). Only results in assay range were considered for statistical evaluation. AMH was also evaluated by ELISA.[DSL-10-14400 active Müllerian Inhibiting Substance/Anti-Müllerian Hormone (MIS/AMH) enzyme-linked immunoabsorbent (ELISA), Diagnostic System Laboratories, Inc., Webster, Tex. 77598-4217, USA], an enzymatically amplified two-site immunoassay, which does not cross-react with other members of the TGF-β super family, including TGF-β1, BMP4 and ACT. Theoretical sensitivity, or minimum detection limit, calculated by interpolation of mean plus two standard deviations of eight replicates of the 0 ng/mL MIS/AMH Standard, was 0.0006 ng/mL. Intra-assay coefficient of variation for an overall average AMH concentration was reported as less than or equal to about 10 percent and in our hands less than about 15 percent. Results are presented in ng/mL, with a conversion factor of 7.14 to pmol/L.

Normal as-AMH and FSH levels have previously been established, based on 95% confidence intervals (CI) at all ages. FIG. 12A.1 and FIG. 12A.2 summarize as-FSH and AMH levels. In the figures, as-FSH and as-AMH levels in candidates are shown against normal as-ranges previously established. As shown in the figures, a considerable number of candidates demonstrate values outside of normal as-range for both, AMH and FSH.

Numbers of triple CGG repeats on the FMR1 gene were evaluated, utilizing commercial assays, as also previously described. In short, FMR1 assays were performed testing DNA by Southern blot and polymerase chain reaction (PCR) to determine size and methylation status of CGG repeats. Southern blot analysis was performed using probe St.B12.3 on EcoR1 and Eagle digested DNA. PCR products were generated using fluorescent labeled primer, and were sized by capillary electrophoresis.

We previously defined and reconfirmed a normal range of CGG repeats as 26 to 34, with median at 30 repeats. Women who's both allele counts fell into this range were considered normal; Those who demonstrated one allele in range and one outside, were considered heterozygous-abnormal, with normal/low (norm/low) and normal/high (norm/high) being separately evaluated. Those with both alleles in abnormal range were considered homozygous-abnormal.

Statistics

All data are expressed as mean+/−standard deviation (SD); a p-value <0.05 was considered statistically significant. Differences between normally distributed variables were tested with analysis of variance or covariance. Differences between groups of variables not conforming to normality were tested for with the Mann-Whitney test. All analyses were carried out with SPSS software for Windows version 17.0, 2005 (SPSS Inc. Chicago, Ill.)

### Results and Discussion of Example 6a

Amongst a total of 164 consecutive egg donor applicants, 73 reached initial AMH evaluations and FMR1 gene analyses and, thus, became donor candidates. As described in Table 7A, their mean age was 24.2+/−3.6 years (range 19-34) and mean AMH was 3.7+/−2.8 ng/mL (range 0.6 to 13.0).

TABLE 7A
Characteristics of donor candidates and donors
Characteristics of donor candidates and donors

| | Candidates | Donors |
|---|---|---|
| | (n = 73) | (n = 21) |
| Age (years; mean ± SD) | 24.2 ± 3.6 | 24.1 ± 3.5 |
| Total oocyte yield | n/a | 17.8 ± 7.2 |
| AMH (ng/mL; mean ± SD) | 3.7 ± 2.8 | 4.2 ± 1.7 |
| as-AMH normal (% of total¹) | 3.0 ± 0.7 (37.0) | 3.2 ± 0.6 (33.3) |
| Oocyte yield² | n/a | 20.7 ± 9.1 |
| low | 1.5 ± 0.5 (26.0) | 1.8 ± 0.2 (14.3) |
| Oocyte yield² | n/a | 15.7 ± 3.8 |
| high | 5.8 ± 2.2 (37.0) | 5.4 ± 1.4 (52.4) |
| Oocyte yield² | n/a | 16.5 ± 6.4 |
| FSH (mIU/mL: mean ± SD) | n/a | 5.9 ± 2.0 |
| as-FSH normal (% of total¹) | n/a | 5.0 ± 1.3 (76.5) |
| high | n/a | 8.5 ± 1.3 (23.5) |
| FMR1: number of CGG repeats | | |
| Allele-1 | 29.1 ± 3.3 | 29.0 ± 3.2 |
| Allele-2 | 32.4 ± 2.2 | 32.2 ± 2.0 |
| Normal³ n (%) | 30 (41.1) | 9 (42.9) |
| AMH (ng/mL; mean ± SD) | 3.3 ± 1.6 | 4.0 ± 1.5 |
| Heterozygous 3 n (%) | 39 (53.4) | 12 (57.1) |
| AMH (ng/mL; mean ± SD) | 3.9 ± 2.7 | 4.0 ± 1.6 |
| Het-norm/low n (%) | 15 (20.6) | 4 (19.0) |
| AMH (ng/mL; mean ± SD) | 3.3 ± 2.3 | 3.9 ± 1.2 |
| Het-norm/high n (%) | 24 (32.9%) | 8 (38.1) |
| AMH (ng/mL; mean ± SD) | 4.4 ± 3.0 | 4.0 ± 1.9 |
| Homozygous³ n (%) | 4 (5.5) | 0 |
| Hom-high/high | 2 (2.7) | — |
| Hom-low/high | 2 (2.7) | — |
| Hom-low/low | 0 | 0 |

The Table summarizes patient characteristics in 73 consecutive egg donor candidates, who after an elimination process (for details see Materials and Methods section) reached a first laboratory testing stage and, from amongst them 21 who reached egg retrieval (one donor did not have FMR1 testing) (donors). Candidates and donors did not differ statistically in any parameter.
¹Reflects percentage of patients in total group.
²AMH does not distinguish between donors with normal or abnormal yields (p = 0.83).
³Normal is defined by both alleles demonstrating between 26-34 CGG repeats; in heterozygous women one allele demonstrates a count outside of this normal range, with het-norm/low defining an abnormally low and het-norm/high and abnormally high count.

Amongst those, 27 (37.0%) demonstrated AMH values within a normal as-range (mean, 3.0+/−0.7 ng/mL); 19 (26.0%) had low as-AMH, suspicious of DOR, (mean 1.5+/−0.5 ng/mL); and another 27 (37.0%) demonstrated abnormally elevated as-AMH levels, suspicious of PCOS (mean 5.8+/−2.2 ng/mL) and, therefore, potentially reflected hyperstimulation risk (FIGS. 12A.1 and 12A.2). Only 17 candidates had baseline FSH levels, and those ranged from 3.1 to 9.8 mIU/mL (FIGS. 12A.1 and 12A.2).

FMR1 analyses amongst candidates demonstrated normal distribution of CGG repeats on both alleles in 30 (41.1%), with 39 (53.4%) being heterozygous abnormal, amongst those 15 (20.6%) being het-norm/low and 24 (32.9%) being het-norm/high. Four candidates (5.5%) were homozygous, two (2.7%) each hom-low/high and hom-high/high but none were hom-low/low. (Table 7A). Overall mean distribution of CGG repeats on both alleles did not vary significantly between candidates and donors (Table 7A, FIG. 12B). FIG. 12B shows the distribution of CGG counts on FMR1 gene in candidates. The differences in distribution were not significant.

Analysis of oocytes yield in 21 candidates who by time of study analysis became donors and, therefore, reached oocytes retrievals allowed limited correlations to FSH, AMH and FMR1 status. Mean age was 24.1+/−3.5 years (range 19-34) and they yielded 17.8+/−7.2 oocytes (range 6-32). AMH levels were 4.2+/−1.7 ng/mL (range, 1.6-7.9) and FSH levels 5.9+/−2.0 mIU/mL (range 3.1-9.8) (Table 7A).

A total of 14/21 (66.7%) of donors demonstrated abnormal as-AMH; amongst those 3/21 (14.3%) abnormally low and 11/21 (52.4%) abnormally high values. Values of as-FSH were abnormally elevated in 4/17 donors (23.5%) and normal in 13/17 (76.5%).

CGG counts were normal in 9/21 (42.9%) and heterozygous-abnormal in 11/21 (57.1%) of donors, 4/21 (19.0%) with heterozygous genotype of norm/low and 8/21 (38.1%) with norm/high (Table 7A).

Investigating abnormalities in laboratory findings as predictors of oocytes yields, 2/21 (9.5%) donors produced abnormally low (<9) oocytes numbers (7 and 6, respectively), 10/21 (47.6%) produced abnormally high oocytes yields (range 16-32) and only 9/21 (42.9%) showed egg numbers considered to be in a normal range of 10-15 (Table 7B). In two donors with abnormally low oocytes production AMH was normal and high, respectively; FSH was available in only one and was normal; and both demonstrated heterozygous abnormal CGG counts, one normal/high and the other normal/low.

Subgroups were too small to reach statistically valid conclusions, cross-tabulating oocytes yields and as-AMH levels. Donors with abnormally high oocytes yields demonstrated in 40.0% normal as-AMH, in 50.0% abnormally high and in 10.0% abnormally low values. Donors with normal oocytes numbers demonstrated normal as-AMH in 22.2%, abnormally high levels in 55.6% and abnormally low as-AMH in 22.2%. AMH statistically did not distinguish between normal and abnormally high oocytes yields (p=0.83).

Six out of seven donors (85.7%) with high oocytes yields demonstrated normal as-FSH, while only 4/7 (57.1%) with normal egg numbers showed normal FSH values and 3/7 (42.9%) elevated as-FSH. These differences were, however not statistically different.

Investigating associations between oocytes yields and CGG counts, all nine women with counts on both alleles in normal range (26-34) demonstrated either normal (6/9, 66.6%) or high (3/9, 33.3%) egg numbers. None had abnormally low oocytes yields. Otherwise, small patient numbers did not allow for further statistically robust conclusions.

In the U.S., the selection of oocytes donors has become increasingly complex since the Food and Drug Administration (FDA) has assumed regulatory authority. The FDA's interests primarily, however, extend to infectious transmission risks. Other aspects of candidate selection are left to patients and physicians. Oocytes (and embryo) yields are, amongst those, of great importance. Correct assessments of OR in oocyte donation candidates represent, therefore, a very essential part of the egg donation process.

A number of perceptive papers recently well summarized progress in assessing OR. Most concentrated on the utilization of AMH, with consensus evolving that AMH may, overall, be a better reflection of OR than baseline FSH. This should not surprise: While AMH and FSH correlate, both, in principle, reflect different stages of follicular maturation, AMH small, preantral and FSH more mature, preovulatory follicles. As OR is defined to reflect remaining follicles in ovaries, the larger preantral pool should, indeed, better reflect OR than preovulatory follicles, primarily represented by FSH.

Though predictive values of FSH and AMH change as females age, and even though as-evaluations offer distinct advantages over nas-testing, OR evaluations still, almost uniformly, utilize nas-normal ranges, independent age. The former, however, also can be expected to improve OR assessments in oocyte donation candidates. This study supports this assumption by demonstrating that a surprisingly large number of them fall outside normal as-OR testing parameters and are, therefore, at risk for either too low or to high oocytes yields. As Table 7A demonstrates, only 37.0 percent of candidates and 33.3 percent of donors demonstrated normal as-AMH, only 76.5 percent of donors normal FSH and only 41.1 percent of candidates and 42.9 percent of donors normal CGG counts.

as-FSH, as-AMH and CGG counts are new tools in defining OR. They never before were applied to selection of oocytes donation candidates. Since oocytes donation is a voluntary and costly process, risks to donors and costs to recipients have to be minimized. Improvements in OR assessments facilitate both: better risk prediction of excessive oocytes yields should eliminate most OHSS risks. Carlesen and associates reported that abnormally high as-AMH denote such risk. At the other extreme, avoidance of disappointing oocytes yields, reduces unnecessary costs.

The frequency of as-AMH abnormalities was surprising. So far, only few studies reported cut-off values suggestive of DOR (and/or poor oocytes/embryo quality) and of OHSS risk. This study utilized as-AMH levels, based on 95% CIs of patients of all ages at our cente. Women with AMH below the lower cut off were considered at risk for DOR; those above the 95% CI for their age were considered at OHSS risk. These levels have previously been demonstrated to discriminate abnormally low and abnormally high oocytes yields.

Remarkably, only approximately one third of donation candidates (37.0%) demonstrated normal as-AMH; approximately a quarter (26.0%) exhibited abnormally low AMH and were, therefore, at risk for low oocytes production; and another remarkable 37.0 percent produced abnormally high levels, placing them at risk for OHSS.

Relatively small donor numbers prevent this pilot study from outright statistical conclusions on specific benefits from here utilized, new OR assessment techniques in oocytes donation cycles. Statistical trends, however, uniformly, and without exception, support a positive effect on donor selection: As Table 7A demonstrates, AMH levels increased from candidates to donors from 3.7+/−2.8 to 4.2+/−1.7 ng/mL. Moreover, AMH levels increased in candidates with normal as-AMH (3.0+/−0.7 to 3.2+/−0.6 ng/mL) and low as-AMH (1.5+/−0.5 to 1.8+/−0.2 ng/mL) but decreased in candidates with high as-AMH who became donors (5.8+/−2.2 to 5.4+/−1.4 ng.mL).

These opposing trends, are, of course, exactly what one would expect as a consequence of better donor selection, with OR improving (improving AMH levels) from candidate status to donor status in women with lower OR and declining (declining AMH) from candidate to donor status in those with excessively high OR.

Though, especially amongst donors, subgroups became too small to reach statistically robust conclusions, correlations with oocytes yields, nevertheless, were remarkable (Table 7B): 9.5 percent demonstrated low oocytes yields (<9), approximately half, high oocytes numbers (16-32) and approximately 40% normal oocytes yield (10-15). Oocytes yields with normal (20.7+/−9.1 oocytes) and high as-AMH (16.5+/−6.4 oocytes) were higher than with low as-AMH (15.7+/−3.8 oocytes), though differences failed to reach significance. The data, however, correlate well with earlier reports that as-AMH discriminates between lower and higher oocytes yields. Table 7B shows the percentage distribution of oocyte yields in donors.

TABLE 7B
Percentage Distribution of Oocyte Yields in Donors
Percentage distribution of oocyte yields in donors

| Oocyte yield | Normal | Low | High |
|---|---|---|---|
| (n oocytes) | 10-15 | (≧9) | (16-32) |
| n donors (%) | 9 (42.9) | 2 (9.5) | 10 (47.6) |
| as-AMH normal (%) | 28.6 | 14.3 | 57.1 |
| low (%) | 66.7 | 0 | 33.3 |
| high (%) | 45.5 | 9.1 | 45.5 |
| as-FSH normal (%)¹ | 57.1 | 100.0 | 85.7 |
| high (%) | 42.9 | 0 | 14.3 |
| FMRI numbers of CGG repeats | | | |
| Normal (%) | 66.6 | 0 | 33.3 |
| Heterozygous² | — | — | — |
| Homozygous² | — | — | — |

¹FSH values did not statistically discriminate between normal and abnormal oocytes yields.
²Numbers too small for statistical evaluation;

The study, therefore, suggests that as-AMH may, indeed, improve egg donor selection. The large number of candidates, by here utilized criteria defined at risk for OHSS, would, however, suggest that as-95% CIs may represent too low a cut off. Nelson et al defined OHSS risk even lower at nas-AMH above 15 μmol/L (2.1 ng/ml). A better definition of OHSS risk, therefore, awaits additional studies, preferably utilizing as-AMH values.

Candidates also demonstrated a significant prevalence of abnormal CGG counts on their FMR1 genes. This should, however, not surprise since Fu et al reported in normal populations a large distribution peak between 29 and 30 repeats but significant additional distribution at lower and higher numbers. We found this peak intriguing and hypothesized that it may represent a normal distribution range in regards to potential OR-related function of the gene.

In this study a little less than half of all candidates (41.1%) and donors (42.9%) showed normal distribution, 53.4 percent of candidates and 57.1 percent of donors demonstrated heterozygous abnormalities. Only candidates (5.5%), but no donors, demonstrated homozygous abnormalities. Donors reaching retrieval, thus, demonstrated a very similar distribution pattern to candidates (Table 7A).

Like general populations, egg donors can, therefore be expected to demonstrate significant CGG count abnormalities. Since such abnormalities may denote different time patterns in decline of OR, such information may be helpful in egg donor selection, especially if donor candidates are in their later 20ies, when FMR1 CGG count patterns can already significantly affect OR.

Here reported oocytes yield data have to be viewed with caution since, like in any retroactive analysis, they already reflect integration of knowledge from earlier screening stages. Some here reported donors, therefore, may have yielded fewer oocytes had they not been recognized as at risk towards DOR, and received appropriately modified stimulation. Integration of earlier OR screening data may, therefore, have possibly blunted otherwise more prominent differences. Similarly, IVF programs integrate clinical suspicion for PCOS into cycle management, whether based on the sonographic appearance of ovaries or high AMH levels.

### Conclusions of Example 6a

In summary, here presented data suggest that as-FSH, as-AMH and FMR1 testing may be helpful in further improving egg donor selection. To what degree, remains to be determined. Such a conclusion is also supported by general advantages of as-testing over nas-OR testing and very predictive patterns of ovarian aging associated with CGG repeats on the FMR1 gene.

### Example 7

### Objective of Example 7

Normal and heterozygous (het) or homozygous (hom) abnormal counts on the FMR1 gene reflect distinct ovarian aging curves. Het abnormal can, however, be normal/low or normal/high, which may affect ovarian reserve (OR) differently.

### Materials and Methods of Example 7

Triple CGG counts on FMR1 gene were investigated in 339 consecutive women. A normal count was 26-34 and FMR1 was considered normal (norm) with both alleles in normal range, het if one allele was abnormal, and hom if both alleles were abnormal. Het may be low/normal (het-1) or high/normal (het-2). Low/normal occurs with one abnormal allele having less than 26 CGG repeats and normal with one normal allele having between 26-34 CGG repeats. High/normal occurs with one abnormal allele having more than 34 CGG repeats and normal with one normal allele having between 26-34 CGG repeats. Whether OR, reflected by anti-Müllerian hormone (AMH) and oocytes yield (phenotype), differs depending on FMR1 genotype, was investigated in 4 groups (norm, het-1, het-2, hom) for groups as a whole, and stratified for age <35 and ≧35 years. Mean ranks between groups were compared with Kruskal-Wallis or Mann Whitney tests.

### Results of Example 7

Among participants in all 4 groups was no difference in AMH (p=0.24) and oocytes (p=0.054). Age-stratified, women <35 years demonstrated, however, significant difference between norm and het-2 and hom and het-1 AMH (Z=−2.22, p=0.027) and oocytes (Z=−3.194, p=0001), not noted above age 35. Het-1 genotype at young age expresses the highest AMH, decreasing rapidly in the early 30s. Het-2, in contrast, presents already at young age with 2^{nd} lowest AMH after hom.

### Conclusions of Example 7

Refinement in CGG count analysis of het abnormal women demonstrates distinct differences in ovarian aging patterns between het-1 and het-2 genotypes. Based on initially very high AMH in het-1 and observed habitués, the het-1 genotype appears to represent (normal weight) women with non-typical PCOS, who at young age unusually rapidly loose OR.

### Example 8

### Objective of Example 8

26-34 triple CGG repeats on the FMR1 gene represent normal, while <26 and >34 repeats increase risk for early decline in ovarian reserve. Effects should, however, vary depending on whether both alleles are in normal (norm) range or either 1 allele [heterozygous (het)] is abnormal or 2 alleles [homozygous (hom)] are abnormal.

### Materials and Methods of Example 8

women (34 egg donors and 305 infertile patients) were evaluated and age-stratified to under (n=153) and above (n=152) age 38 years. Box and whisker plot confirmed 26-34 CGG counts as normal (median 30). A woman was either norm (both alleles in range), het or hom (1 or 2 alleles outside range). Ovarian reserve was defined by anti-Müllerian hormone (AMH), follicle stimulating hormone (FSH) and oocytes yield.

### Results of Example 8

Both patient groups were significantly older than donors (p<0.001), but donors only trended towards higher AMH levels (p=0.071). They did not differ in AMH based on CGG counts. Patients under 38 decreased AMH from norm to het and hom (p=0.032) (norm/hom, p=0.009; normll het, p=0.063), showed lower oocytes yields (p=0.018), though no FSH changes. Donors and patients under 38 years combined (AMH, p=0.027; oocytes yield p=0.024), but differences were lost above 38. At young ages linear regression showed higher AMH in norm, crossing het at approximately age 35, after which, het have higher AMH. Norm and hom regressions cross in patients in their late forties. Patterns were also confirmed in binned age analysis.

### Conclusions of Example 8

Ovarian aging differs based on norm, het or hom CGG counts, confirming an association between CGG repeats and ovarian reserve, and suggesting an FMR1 effect on follicular recruitment in favor of follicular preservation and fertility extensions into older age. Such functions potentially contribute to species maintenance, which may explain why the FMR1 gene is highly preserved despite obvious, and at often severe medical consequences primarily in males.

As stated herein, and supported at least by the examples herein, fertility profiling, based on triple repeat numbers and autoimmune abnormalities will benefit women with already established infertility. Further, this fertility profiling will provide females with the ability to predict premature decreases in ovarian function (and therefore female infertility) and facilitate early diagnosis of subfertility, which in turn, would allow women to adjust reproductive planning and/or to take fertility preserving steps, like oocyte, ovarian, or embryo cryopreservation. A fertility profile, consistent of autoimmune testing and assessment of triple CGG repeats on the FMR1 gene, may thus become a universal screening test for the fertility potential of young women.

### Example 9

### Objective of Example 9

The FMR1 gene partially appears to control ovarian reserve, with a specific ovarian sub-genotype statistically associated with a polycystic ovary (PCO)-like phenotype. Some forms of PCO have been associated with autoimmunity. We, therefore, investigated in multiple regression analyses associations of ovary-specific FMR1 genotypes with autoimmunity and pregnancy chances (with in vitro fertilization, IVF) in 339 consecutive infertile women (455 IVF cycles), 75 with PCO-like phenotype, adjusted for age, race/ethnicity, medication dosage and number of oocytes retrieved. Patients included 183 (54.0%) with normal (norm) and 156 (46%) with heterozygous (het) FMR1 genotypes; 133 (39.2%) demonstrated laboratory evidence of autoimmunity: 51.1% of het-norm/low, 38.3% of norm and 24.2% het-norm/high genotype and sub-genotypes demonstrated autoimmunity (p=0.003). Prevalence of autoimmunity increased further in PCO-like phenotype patients with het-norm/low genotype (83.3%), remained unchanged with norm (34.0%) and decreased in het-norm/high women (10.0%; P<0.0001). Pregnancy rates were significantly higher with norm (38.6%) than het-norm/low (22.2%, p=0.001). FMR1 sub-genotype het-norm/low is strongly associated with autoimmunity and decreased pregnancy chances in IVF, reaffirming the importance of the distal long arm of the X chromosome (FMR1 maps at Xq27.3) for autoimmunity, ovarian function and, likely, pregnancy chance with IVF.

### Materials and Methods of Example 9

This study involved the review of medical records. Data utilized was extracted from medical charts and the Center's centralized, confidential electronic research data bank. Patients, at time of initial consultation, sign an informed consent, which permits such utilization of medical records for research purposes.

Patients/Cycles

We investigated 339 consecutive female infertility patients who presented to our center for initial diagnostic evaluation, which routinely includes limited genetic and immunologic testing. Amongst those, 75 women qualified as PCO-like phenotypes, based on large numbers of oocytes retrieved (greater than or equal to about 12) in first IVF cycles, and/or high anti-Müllerian hormone (AMH, greater than about 4.0 ng/mL) at initial evaluations. These values were chosen in consideration of the high prevalence of diminished ovarian reserve in our patient population (see also ovarian reserve tests in Table 8).

TABLE 8
Patient Characteristics*

| | Ovarian Phenotype | | | | |
|---|---|---|---|---|---|
| | Normal | | PCO-like | | P-Value |
| Number | 264 | | 75 | | |
| Age (years) | 38.8 ± 4.8 | | 34.8 ± 4.3 | | <0.0001 |
| FSH | 10.3 | (9.72-11.03) | 7.35 | (6.67-8.08) | <0.0001 |
| (mIU/Ml, 95% CI) | | | | | |
| AMH | 0.55 | (0.48-0.62) | 2.21 | (1.76-2.77) | <0.0001 |
| (ng/mL, 95% CI) | | | | | |
| BMI | 22.7 ± 13.5 | | 23.3 ± 13.2 | | 0.74 |
| | | | | | (N.S.) |
| Oocytes | 4.8 ± 3.3 | | 17.5 ± 6.7 | | <0.0001 |
| retrieved (n) | | | | | |
| Embryos | 2.4 ± 1.1 | | 2.4 ± 0.8 | | 0.88 |
| transferred (n) | | | | | (N.S.) |
| Ethnicity/Race | | | | | |
| (n/%) | | | | | |
| Caucasian | 157 | (59.5) | 43 | (57.3) | |
| African | 33 | (12.5) | 12 | (16.0) | |
| Asian | 38 | (14.4) | 10 | (13.3) | |
| Middle Eastern | 11 | (4.2) | 2 | (2.7) | |
| Ashkenazi Jewish | 15 | (5.7) | 6 | (8.0) | |
| Other | 10 | (3.8) | 2 | (2.7) | |
| Autoimmunity | | | | | |
| (n/%) | | | | | |
| No | 162 | (61.4) | 43 | (57.3) | N.S. |
| Yes | 102 | (38.6) | 32 | (46.7) | |

*Ovarian reserve tests in this table reflect first patient evaluations. IVF outcome data reflect only first IVF cycles. Data are shown with confidence intervals when log-transformed for analysis and with standard deviations where not. Positive autoimmunity denotes sum of all positive patients, demonstrating at least one abnormality in the immune panel tested (for further details, see Methods section).

The 339 patients underwent 455 IVF cycles, 116 thus representing repeat attempts. As shown in Table 8A, however, demonstrates, adding a covariate for number of IVF cycles experienced by each patient in our patient population does not affect the findings of an analysis based on per patient outcomes.

TABLE 8A
Pregnancy analysis of only 339 1^{st} IVF cycles*

| | Norm | Het-norm/low | Het-norm/high |
|---|---|---|---|
| Pregnant [n (%)] | 54 (29.5) | 14 (14.9) | 15 (24.2) |
| Not pregnant | 129 (70.5) | 80 (85.1) | 47 (75.8) |

*Chi-Square 7.17; df = 7.17; P = 0.028

Genetic testing includes an assessment of triple CGG nucleotide repeats on the FMR1 gene with regard to ovarian function, 26 to 34 repeats represent a normal range (median 30), and counts below and above denote risk towards POA/OPOI. Based on this normal range, normal (norm), heterozygous (het) and homozygous (hom) FMR1 genotypes can be described, which are reflective of distinct ovarian aging patterns. They are defined by both alleles in normal range (norm), either one outside of range (het) or both alleles outside of range (hom). A het patient, in turn, can be either het-norm/low (<26 repeats) or het-norm/high (>34 repeats), while hom patients may be either high/high, high/low or low/low.

The het-norm/low FMR1 genotype has in a longitudinal and cross-sectional study been associated with very high ovarian reserve (based on AMH) at young ages, which, however, in the early 30s, quickly depletes, resulting in rapid AMH declines at relatively young ages and relatively diminished age-dependent ovarian reserve thereafter. Women with this genotype after ages 32-33 years, therefore, lose their PCO-like phenotype and, without FMR1 evaluations, based on AMH levels alone, would be perceived as either normal or suffering from prematurely diminished ovarian reserve.

Immunological testing involves total immunoglobulin levels (IgG, IgM, IgA), antinuclear, anti-phospholipid and anti-thyroid antibody panels, as well as anti-ovarian and anti-adrenal antibodies. We previously demonstrated that this panel of immunological tests is sufficient in identifying even subclinical levels of autoimmunity that predisposes towards POA/OPOI. In utilizing this screening method, a patient was defined as autoimmune-negative with absence of any abnormality and was considered autoimmune-positive with presence of even one, fully recognizing that such a definition increases sensitivity at expense of specificity and, therefore, weakens the discovery of potentially existing associations with autoimmunity. This definition of autoimmunity, therefore, consciously biases results against positive association and, thus, strengthens any detected associations.

IVF cycles were conducted in routine fashion. In principle, only two ovarian stimulation protocols were utilized: women with normal ovarian reserve were down-regulated with a gonadotropin releasing hormone agonist and stimulated with maximally 300 IU of gonadotropins daily. Patients with diminished ovarian reserve received a micro-dose agonist protocol with about 450 to about 600 IU of gonadotropins daily. Hormone assays for follicle stimulating hormone (FSH), estradiol and AMH were tested at our center.

Statistical Analysis

Patient characteristics and IVF cycle outcomes were evaluated in association with FMR1 genotypes and autoimmune status, as defined above. PCO-like phenotypes were distributed in 62 percent as norm, 25 percent as het-norm/low and 13 percent as het-norm/high and represented 22 percent of all patients. Women with normal ovarian phenotype were in same order distributed at 52, 28 and 20 percent, respectively and represented 78 percent of all patients.

This distribution corresponds to an effect size (w) of 0.091 and, equivalently, to a contingency coefficient (C) as well as a Cramer's phi coefficient (phi) of 0.091. With a sample size of 339, the study had a power of 30.3% to yield statistically significant results. Assuming continuation of this study in observed proportions, a study with identical effect size (w=0.091) would require a sample size of 1,164 patients to achieve an 80 percent power to detect a significant alpha (0.05, two-tailed).

Univariate comparison between women with PCO-like phenotype and control was performed using Chi Square and analysis of variance as appropriate. Variables in which the distribution of data did not conform to normality were first log transformed for analysis and then converted back to standard units for presentation. Where cycles served as study units, odds ratios were compared. Continuous variables are presented as either mean±standard deviation (SD) or mean and 95 percent confidence interval (95% CI), as appropriate.

We constructed a general linear model with logit link function, testing the association of the PCO-like phenotype with FMR1, and autoimmunity and also for pregnancy. General linear models were run on SAS version 9.2 (GENMOD module).

### Results of Example 9

Table 8 (above) summarizes patient characteristics for all 339 women, amongst those 264 (77.9%) representing a normal, non-PCO-like ovarian phenotype and 75 (22.1%) the defined PCO-like phenotype. Women with PCO were younger (34.8±4.3 vs. 38.8±4.8 years, P<0.0001), had lower FSH (8.0±4.0 vs. 11.9±6.8 mIU/mL, P<0.0001) and higher AMH levels (3.2±2.6 vs. 0.8±0.7 ng/mL P<0.0001) and produced larger oocyte yields (17.5±5.6 vs. 4.8±3.3, P<0.0001). Otherwise, the two groups did not differ significantly, including in ethnic/racial distribution and BMI, confirming the non-obese nature of the here investigated PCO-like phenotype.

FMR1 Genotypes

Table 9 summarizes FMR1 genotype distribution patterns in women with apparently normal and PCO-like ovaries. Amongst 264 women with normal ovaries 137 (51.9%) demonstrated a norm FMR1 genotype; the remaining 127 (48.1%) were het (there were no hom patients in the study population), 52 (19.7%) were het-norm/high and 75 (28.4%) het-norm/low. This distribution did not significantly differ from the FMR1 genotype distribution amongst the 75 women with PCO-like phenotype, where norm were 46 (61.3%), het-norm/high 10 (13.3%) and het-norm/low 19 (25.3%). The age-dependency of the definition of the PCO-like phenotype mandates caution in interpreting these results.

TABLE 9
FMR1 genotype distribution*

| Phenotype | Norm | Het-Norm/High | Het-Norm/Low |
|---|---|---|---|
| PCO-like | 46 (61.3) | 10 (13.3) | 19 (25.3) |
| Normal | 137 (51.9) | 52 (19.7) | 75 (28.4) |
| Total | 183 (54.0) | 62 (18.3) | 94 (27.7) |

*Chi-Square 2.46, df = 2.0, P = 0.29.

Autoimmunity

Distribution of autoimmunity also did not differ between both patient groups (Table 8): In women with normal ovaries 162/264 (61.4%) showed no evidence of autoimmunity and 102 (38.6%) did, while in the PCO-like cohort 43 (57.3%) did not and 32 (46.7%) did.

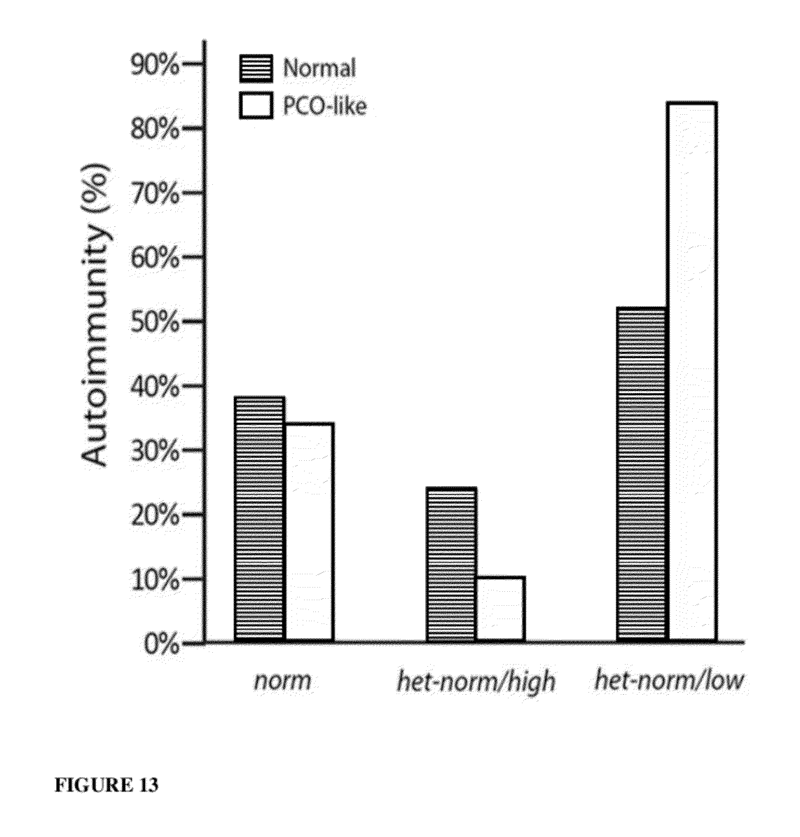

If autoimmunity was, however, assessed in reference to FMR1 genotype (FIG. 13—Prevalence of Autoimmunity in Reference to FMR1 Genotype), the het-norm/low genotype was most frequently associated with autoimmunity (51.1%), followed by norm patients (38.3%) and het-norm/high women (24.2%), a statistically significant difference in distribution (p=0.003). These differences in distribution further strengthened in women with PCO-like phenotype: het-norm/low women demonstrated autoimmunity in 83.3 percent of cases, while het-norm/high genotypes in only 10.0 percent, almost categorically differentiating between these two het genotypes, while norm women held a middle ground with 34.0 percent prevalence (P<0.0001).

The prevalence of autoimmunity was in both patient groups the highest with het-norm/low FMR1 genotype and the lowest with het-norm/high genotype. This pattern, however, intensified in women with PCO-like phenotype. Gray bars represent women with normal ovarian reserve; white bars represent the PCO-like phenotype.

IVF Pregnancy Rates

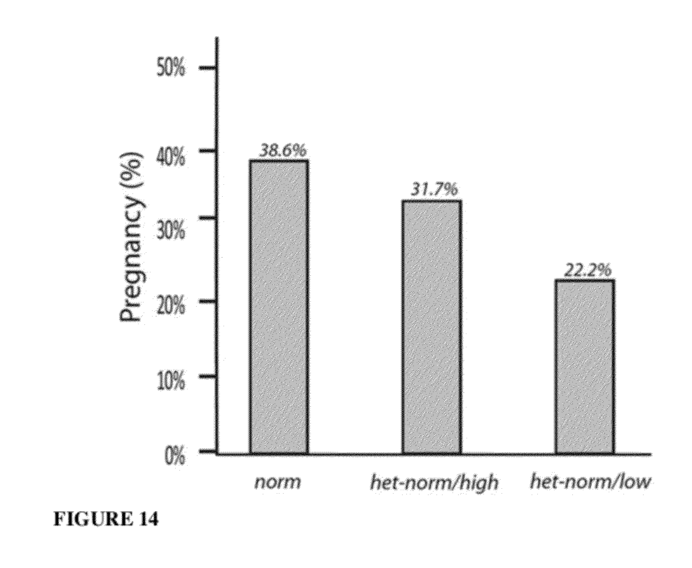

Pregnancy outcomes in 455 consecutive IVF cycles are summarized in FIG. 14 (Pregnancy Rates in IVF Based on FMR1 Genotype). As the figure demonstrates, norm women experienced the highest pregnancy rates (38.6%), a rate significantly higher than in het-norm/low patients [22.2%; OR 0.84 (95% CI 0.74 to 0.96; Wald 6.9; df=1; P=0.009)]. Women with het-norm/high genotype had intermediate pregnancy rates at 31.7 percent. Adjustment for age maintained the disadvantage in pregnancy rate for het-norm/low women (OR 0.43; 0.22 to 0.86, p=0.017).

Pregnancy rates were the highest with norm FMR1 genotype and the lowest with het-norm/low genotype.

### Discussion of Example 9

This study demonstrates that in consecutive patients presenting for infertility treatments the prevalence of autoimmunity varies significantly with FMR1 genotype, with het-norm/low presenting with most and het-norm/high with least autoimmunity. This distribution is further strengthened in infertile women with the lean PCO-like phenotype, as the het-norm/low FMR1 genotype is associated with relatively rapidly depleting ovarian reserve. Such patients almost guarantee positive autoimmune laboratory findings (83.3% prevalence, FIG. 13), while a het-norm/high genotype is practically protective against autoimmunity (10.0% prevalence). Women with norm FMR1 genotype in both patient populations take up a middle ground with 38.3% and 34.0% prevalence, respectively.

The close association between the het-norm/low genotype and autoimmunity is further supported by the fact that the PCO-like phenotype was significantly younger (P <0.0001, Table 8), while autoimmunity actually increases in prevalence with advancing female age. PCO-like phenotypes, thus, demonstrated significantly more autoimmunity, despite significantly younger ages.

The statistical clarity of reported results here is, however, especially remarkable, considering that patient selection criteria in this study strongly biased against discovery of such statistical associations. As already previously noted, the definition of positive autoimmunity consciously was based on improving sensitivity at the expense of specificity. Women defined as autoimmune, therefore, likely included a few without real polyclonal autoimmune activation.

Even more significantly, however, the time line for premature declines in ovarian reserve in women with the het-norm/low FMR1 genotype. Even though the here investigated group of patients with PCO-like phenotype were significantly younger (P<0.0001, 8), it appears likely that older women with apparently normal ovarian phenotype must include at least some who at younger ages actually did demonstrate a PCO-like phenotypes.

Patients with PCO-like phenotype, in this study defined by about 12 or more oocytes retrieved and/or an AMH above about 4.0 ng/mL, represented about 75 (22.1%) of all patients investigated. The definition of the PCO-like phenotype in this study was purely clinical, meant to identify a patient population with disproportionally high ovarian reserve, as documented by high oocyte yields and AMH values. Considering that the here investigated patient population included a disproportionate number of women with significantly diminished ovarian reserve (confirmed by elevated FSH, low AMH and oocyte yields in women with normal ovarian phenotype, Table 8), here chosen cut offs, defining a PCO-like phenotype for study purposes, appear appropriate. Twelve or more oocytes in such patients are above expected averages, as even women under about age 35 years at our center produce only an average of 8.2+5.8 oocytes. Similarly, an AMH above about 4.0 ng/mL exceeds the 95% confidence interval (CI) of AMH levels at our center in women as young as about age 26 years.

Here reported autoimmune laboratory findings in slightly above one third of women correspond well to prevalence numbers for infertility populations. This validates the selected study population and also reaffirms the immune profile used to define presence of subclinical levels of autoimmunity. Though not reaching significance, prevalence of autoimmunity further increased (38.6% to 46.7%) from normal ovarian to PCO-like phenotypes. As further discussed herein, there is a strong association between het-norm/low FMR1 genotype and autoimmunity.

To define autoimmunity at subclinical levels is difficult and is the reason why clinical diagnoses of autoimmune conditions in prodromal stages may be difficult. Autoimmunity is, however, typically associated with a polyclonal activation of the immune system, which can be detected by broadly based laboratory evaluations. While such screens are not specific enough for diagnoses of autoimmune diseases, they appear sensitive enough in defining evidence of autoimmune activity.

This study reaffirms a surprisingly close association between autoimmunity and the het-norm/low FMR1 genotype. We previously associated the het-norm/low genotype in a pilot study with a PCO-like phenotype, with rapidly depleting ovarian reserve. Women with this genotype present at young ages with a PCO phenotype. Because of rapid follicle depletion (i.e., rapidly diminishing ovarian reserve), they then at older ages demonstrate normal to abnormally low AMH levels, reflecting relative or outright diminished ovarian reserve.

Since the FMR1 gene appears closely involved with regulation of follicle recruitment and, therefore, ovarian reserve, the association between het-norm/low FMR1 genotype and PCO-like phenotype makes sense. The extremely close association between het-norm/low genotype and autoimmunity was completely unanticipated. This association appears, indeed, so close that in a PCO-like population a het-norm/low genotype virtually predicts autoimmunity, while women with the het-norm/high genotype appear protected from autoimmunity.

These associations also translate into clinical significance for infertile women, since the FMR1 genotype appears predictive of pregnancy chances with IVF. Women experience best pregnancy chances with norm, intermediate chances with het-norm/high and lowest rates with het-norm/low genotypes (FIG. 2).

Whether a PCO-like phenotype, alone, affects pregnancy chances in IVF has been discussed. Lower and similar pregnancy rates have been suggested in PCOS in comparison to other infertile patients undergoing IVF. Multiple underlying etiologies for PCOS, different ovarian stimulation protocols and variability in genetic definitions of study populations easily explain such discrepancies.

The same kind of discussion surrounds the association of autoimmunity and pregnancy success in IVF. Many authorities have categorically denied an association, while others have pointed at considerable evidence. Here presented data suggest a possible explanation for these contradictory opinions since, like PCOS, most studies on autoimmunity have been performed in genetically and etiologically heterogenic patient populations. At least in a genetically homogenous population of women with the het-norm/low FMR1 genotype, autoimmunity, indeed, appears negatively associated with pregnancy chances in IVF.

Autoimmunity is abnormally high in practically all X-linked disorders. If defective, a MHC-paralogue on the long arm of the X chromosome renders individuals immunologically less efficient. With the FMR1 gene mapping to Xq27.3, it appears to occupy the cross roads between ovarian function (ovarian recruitment and ovarian reserve) and autoimmunity.

Both autoimmunity and abnormalities in ovarian reserve are closely associated with X chromosome defects: A good example is Turner syndrome, in which Xq21 terminal deletions are common, often large and characterized by primary as well as secondary amenorrhea. Xq21 or further distal deletions usually present with secondary amenorrhea, a classical clinical presentation of premutation range FMR1 (fragile X) carriers who present with POF/POI. In contrast Turner syndrome with normal fertility generally involves more proximal Xq deletions.

POF/POI, indeed, demonstrates a 4 MB locus exactly at Xq27-q28, with the FMR1 gene mapping to Xq27.3. Small deletions in Xq27-q28 have variable phenotypes, some with early menopause but are usually able to reproduce until experiencing full POF/POI. Similar correlations with ovarian function are also observed in balanced translocations, where only Xq23-q27 deletions are associated with POF/POI.

Turner syndrome is not only characterized by above noted abnormalities in ovarian function but, like most X-linked disorders, also by excessive autoimmunity. Both autoantibodies and autoimmune diseases are significantly increased. The close association between X-linked disorders and autoimmunity led to the suggestion that the latter may be the consequence of genes and/or mutations on the long arm of the X chromosome, potentially explaining the increased prevalence of autoimmunity in women in comparison to males.

A PCO-like phenotype with strongly associated autoimmunity and specific FMR1 genotype (het-norm/low), and the protective effect of another FMR1 genotype (het-norm/high), support the notion that the FMR1 gene plays a role in regulating ovarian reserve. Now it appears that the same gene may also be involved in determining risk/protection of/from autoimmunity.

These observations also raise the intriguing possibility that a PCO-like phenotype may be associated with an autoimmune etiology. This has previously been suggested but, in contrast to cases of POF/POI, PCO and/or PCOS have never been established as autoimmune in nature. Like POF/POI in its various forms, PCOS is multifactorial in etiology, with PCO being a unifying phenotypical presentation of an, otherwise, still very controversial syndrome.

Serological evidence for elevated autoimmunity in women with PCOS exists and investigations of non-organ specific (antihistone and anti-dsDNA) antibodies were completed. Also, investigations of non-organ specific (antihistone and anti-dsDNA) were completed here. Statistical associations between levels of anti-thyroid peroxidase antibodies and treatment response in women with PCOS suggest that organ specific thyroid antibodies may also be associated with PCOS. Thyroid autoimmunity, of course, is well known to be closely associated with ovarian autoimmunity.

The here presented data support the hypothesis that, like other endocrine organs, ovaries may be subject to suppressive and stimulatory autoimmune influences, possibly mediated by autoantibodies. “Functional” autoantibodies may represent a universal paradigm of autoimmunity.

In regards to ovarian function such a concept would suggest inhibitory autoantibodies as cause of selected cases of POA/OCPOI and POF/POI and stimulatory autoantibodies as promoters of follicular activity, resulting in at least one PCO-like phenotype. The here reported strong positive and negative associations with both het-FMR1 genotypes further suggest a possible role of the FMR1 gene in regulation of these “functional” autoantibodies and, therefore, possibly, in contributing to the well established higher prevalence of autoimmunity in females in comparison to males.

### Example 10

### Objective of Example 10

FMR1 as well as BRCA genes may affect ovarian reserve. Based on a normal range of 26-34 (median 30) CGG repeats, different ovarian FMR1 genotypes and sub-genotypes have been described and have been associated with varying ovarian aging patterns. While the normal range of CGG repeats is constant, prevalence of genotypes and sub-genotypes varies between races. They also affect pregnancy chances in association with in vitro fertilization (IVF), and define risk towards autoimmunity in infertile women.

Ovarian effects of BRCA genes are less well defined: BRCA1 but not BRCA2 has been associated with occult primary ovarian insufficiency. This commonality between BRCA and FMR1 led us to investigate associations between FMR1 and BRCA1/2.

Method and Materials of Example 10

Study Groups

The study involved two distinct patient populations: (i) 99 female BRCA1 or BRCA2 mutation positive breast cancer patients. Their BRCA1/2 testing was performed at the Medical University Vienna, Vienna, Austria, while their FMR1 assays were performed at the Medical University Graz, Graz, Austria. (ii) 410 female infertility patients, whose anonymized clinical information, including FMR1 testing results, were stored in the electronic research data base of the Center for Human Reproduction in New York, U.S.A.

Coordination of research efforts between Austrian and U.S. centers was involved, such that the BRCA1/2 and FMR1 data of the Austrian breast cancer patients could be statistically analyzed, anonymized and in bulk.

When an initial statistical analysis determined the presented data here, questions arose about compatibility of Austrian and U.S. FMR1 results. The Austrian laboratory was, therefore, requested to provide random anonymized results of a patient population reflecting the whole CGG spectrum. FMR1 genotypes and sub-genotypes of 105 such patients did not differ significantly in either median or distribution between 25^{th} centile and 75^{th} centile from infertile U.S. controls.

Laboratory Analyses

As noted, BRCA1/2 and FMR1 analyses of the Austrian study group were performed in Vienna (BRCA1/2) and Graz (FMR1), respectively. Until and inclusive of 2008, BRCA1/2 analyses were performed by denaturation high performance liquid chromatography, as previously reported by the laboratory. After 2008 DNA sequencing, with use of chain-terminating inhibitors was utilized.

FMR1 analyses in Austria were performed as previously in principle reported from the New York laboratory. In brief, DNA-concentration and purity were determined by measurements with a NanoDrop ND-1000 Spectrophotometer. A Fragile X PCR Screen Kit (Abbot Molecular, Vienna, Austria) was utilized to detect expanded CGGₙ in the 5′ untranslated region of FMR1. PCR was set up in total volume of 20μ (13 μl High GC PCR Buffer; 0.8 μl FMR1 Primers-2; 1.2 μl TR PCR Enzyme Mix; 2 μl DNase/RNase free water; and 3 μl genomic DNA at concentration of 10 mg/μl) and performed with a PTC200 Thermocycler (Biorad, Vienna, Austria; Cycling conditions: 98.5° C. for 30 seconds; 53° C. for 30 seconds and 75° C. for 1 minute with 50 cycles). PCR products were stored at −20°.

Capillary electrophoresis was performed by mixing 2 μl of PCR product with 20 μl Hi-Di™ formamide (Applied Biosystems, Foster City, Calif., USA) and 2 μl of ROX™ 1000 Size Standard (supplied with above noted Fragile X Kit), and incubating at 95° for 2 minutes to denature the DNA, followed by immediate cooling to 25°. Samples were then loaded into an ABI 3100 Sequencer (Applied Biosystems), using a 36 cm array and POPE polymer. Injection and run conditions were set according to protocol, provided with above noted Fragile X Kit. GeneMapper software v3.5 (Applied Biosystems) and electropherograms were used to determine CGGₙ.

New York FMR1 analyses were performed by commercial assays, as previously reported. In brief, DNA analysis was performed by PR amplification, followed by agrose gel, as well as capillary electrophoresis and Southern blot analysis, using the probe StB12.3 and restriction enzymes EcoR1 and Eag1.

Austrian FMR1 data were reported for both alleles as CGGₙ, and in New York converted to the format of ovarian genotypes and sub-genotypes. In brief, it is based on a normal range of CGGₙ₌₂₆₋₃₄, with median of 30 repeats. Women, therefore, can have the following genotypes: normal (norm) if both alleles are in normal range; heterozygous (het), if one allele is in and one outside of normal range; and homozygous (hom) if both alleles are outside normal range. Het and hom genotypes can then be further subdivided, depending whether abnormal alleles are above (high) or below (low) normal range into het-norm/high and het-norm/low and hom-high/high, hom-high/low and hom-low/low sub-genotypes. Because of the small number of hom patients, they are not sub-divided into sub-genotypes in this study.

Statistical Analysis

Proportions of FMR1 genotypes and sub-genotypes were compared between the two study groups using cross-tabulations and calculations of Chi-square and Cramer's V statistics. When comparing CGGₙ as a continuous function between the groups, nonparametric testing was used because CGGₙ in populations tends to be positively skewed. Mann-Whitney U tests were conducted to evaluate differences between the two groups on median change in CGGₙ of both alleles.

All statistical calculations were performed utilizing SPSS, version 18 (Chicago, Ill.).

Results and Discussion of Example 10

The study involved two distinct patient populations: (i) 99 Austrian, BRCA1/2—positive breast cancer patients; (ii) 410 U.S. based infertility patients, who underwent FMR1 testing and served as comparison population [a similar infertility population previously was shown to demonstrate similar CGG count distributions to a general population].

Austrian and U.S. Institutional Review Boards (IRBs) approved investigation of respective patient populations.

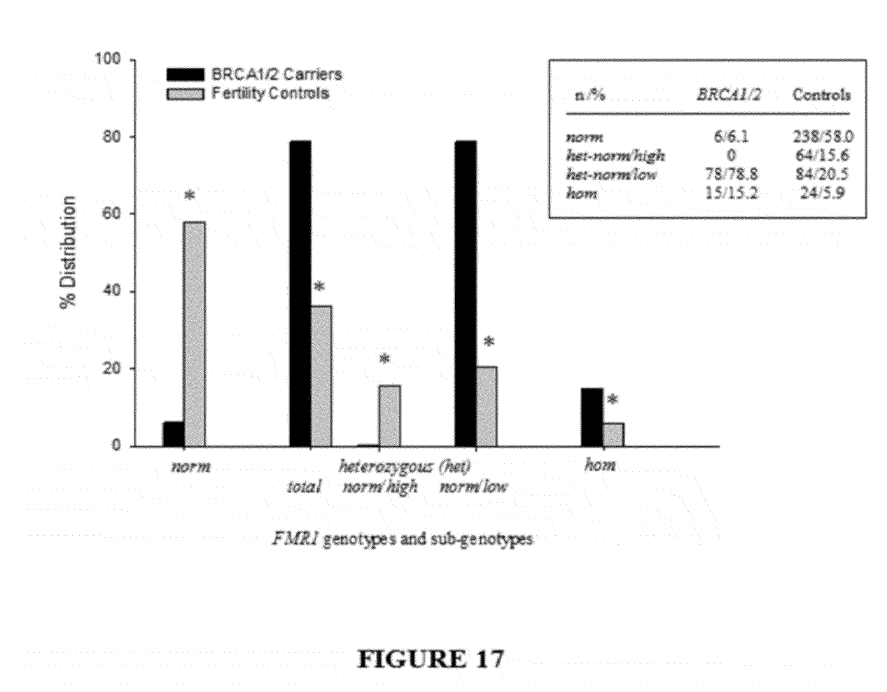

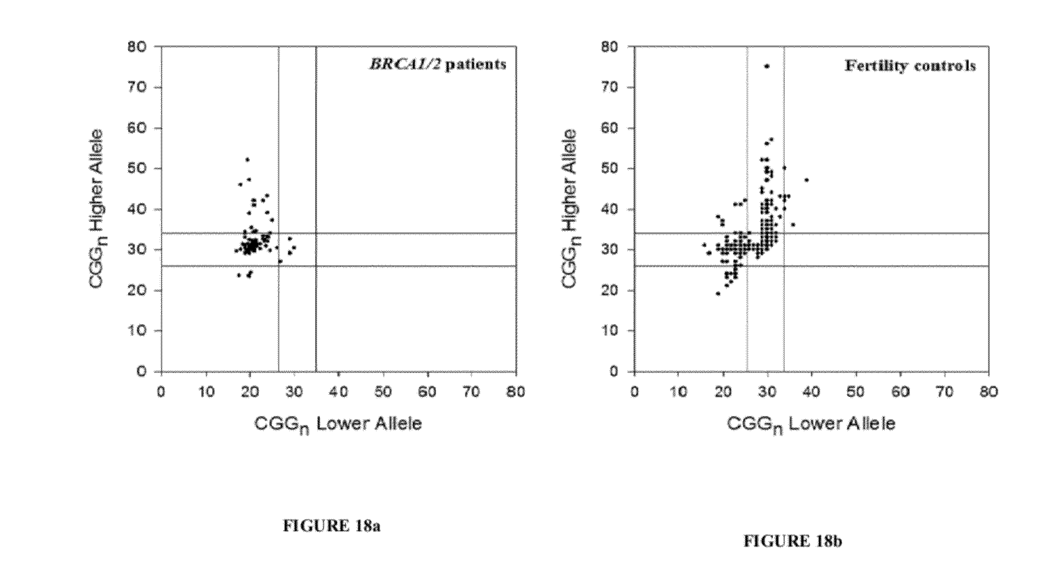

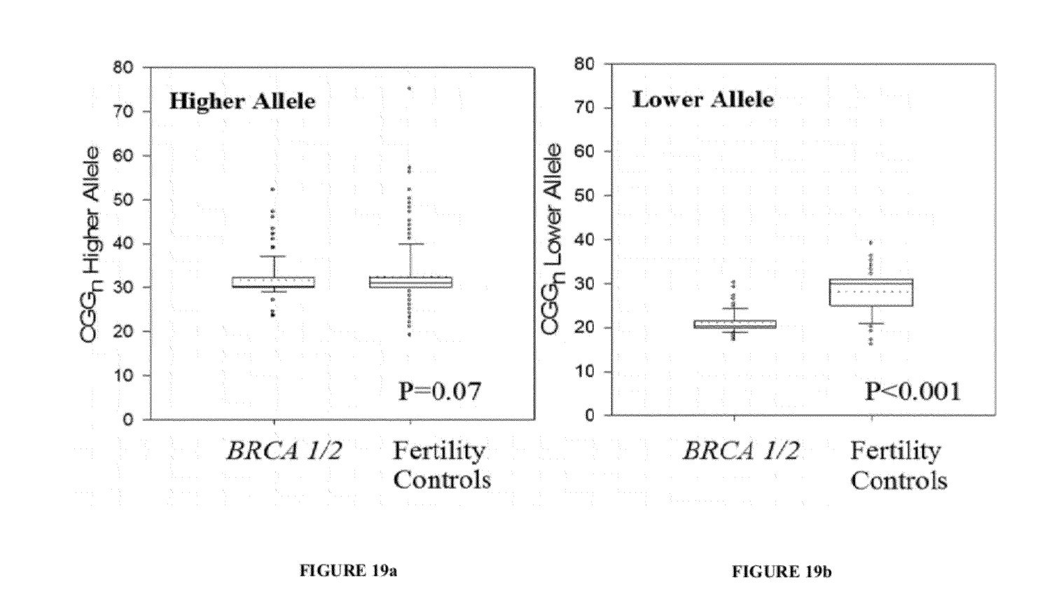

FIG. 17 shows the distribution of FMR1 genotypes and sub-genotypes in women with BRCA1/2 mutations (black bars) and U.S. (gray) comparison group are shown in FIG. 17; the “*” within each category denotes significance at P<0.05. Noteworthy are the excess of het-norm/low and complete absence of het-norm/high in FMR1 sub-genotypes in BRCA1/2 mutation carriers, and the very low prevalence of women with norm FMR1 genotype. A numerical presentation of these data is presented in FIGS. 18 and 19. In these figures, genotypes “norm” stands for normal, “het” is for heterozygous and “hom” is for homozygous, and further, in description of sub-genotypes, high stands for CGG n>34, and low for CGG n<26.

Infertile control patients demonstrated similar FMR1 genotypes and sub-genotype distribution as reported before in such populations (FIG. 17), with normal (norm), heterozygous (het) and homozygous (hom) genotypes of 58.0, 36.1 and 5.9 percent, respectively. The expected distribution was also observed with sub-genotypes, with het-norm/high at 15.6 percent and het-norm/low at 20.5 percent. This distribution of CGGₙ also follows that in general populations.

In contrast, as FIG. 17 also demonstrates, BRCA1/2-positive women presented with substantially different FMR1 genotype and sub-genotype distributions: A large majority of BRCA1/2 women were found to exhibit the het-norm/low FMR1 sub-genotype (74.0% BRCA1 and 83.7% BRCA2, respectively). Combined, 78.8 percent of women who were BRCA1 or BRCA2 positive exhibited the het-norm/low FMR1 sub-genotype. Moreover, in stark contrast to controls, no BRCA1/2 carriers, at all, demonstrated the het-norm/high FMR1 sub-genotype.

Similarly, BRCA1/2-positive patients demonstrated almost no norm genotypes, otherwise the most frequently represented single genotype in the comparison population (FIG. 17): Only 10.0 and 2.0 percent of BRCA1 and BRCA2 patients, respectively (combined 6.1%), demonstrated a norm FMR1 genotype.

The hom genotype was mildly overrepresented in BRCA1 and BRCA2 carriers (16.0% and 14.3%, respectively; combined, 15.2%). Numbers were too small for meaningful assessments of sub-genotypes.

In comparing distribution of FMR1 genotypes and sub-genotypes between BRCA1/2 patients and U.S. controls (with hom patients collapsed into one group), group membership was significantly related [x² (6, N=614)=158.71; P<0.0001).

As shown by cross tabulations, there were significant relationships between group membership and FMR1 genotypes/sub-genotypes. Non-parametric testing (Mann-Whitney U test)) confirmed statistically significant differences in median change for CGGₙ on the low count allele of the FMR1 gene between both patient groups, with follow up tests (Dunn's Method) indicating significant differences between groups (See FIGS. 18a, 18b, 19a and 19b showing the distributions of individual CGGₙ in all three patient populations).

FIGS. 18a and 18b show the distribution on both FMR1 alleles, of CGGₙ in BRCA1/2 mutation carriers as well as U.S. controls in the form of scattergrams. Horizontal and vertical parallel lines in scattergrams define the norm distribution area (CGGₙ₌₂₆₋₃₄), with areas below and above representing low and high, sub-genotypes, respectively; FIGS. 19a and 19b represent higher and lower count allele, respectively, for individual patients. Only the lower count allele varied significantly between the two groups (Mann-Whitney U test, P<0.001). FIGS. 18b and 18b scattergrams, as well as FIGS. 19a and 19b, demonstrate graphically the significant shift towards low FMR1 sub-genotypes, especially on the lower count allele of BRCA1/2 mutation carriers. In FIGS. 19a and 19b, the dashes ( - - - ) represent the mean, and the solid (_) represents the median.

For the lower CGGₙ allele, in most cases representative of a low FMR1 genotype/sub-genotype, values amongst the two groups were significantly different [Mann-Whiney U=(Mean Rank 83.37_{low}, 296.44_{high}; Z=−13.10; P=0.001)].

Similarly, the higher count CGGₙ allele, mostly representing high FMR1 genotypes/sub-genotypes, varied amongst the two groups (Mann-Whitney U=Mean Rank 231.18_{low}, 260.75_{high}; Z=−0.069; P=0.07) but failed to reach statistical significance (see FIGS. 19a and 19b).

The most likely explanation for complete absence of the het-norm/high ovarian FMR1 sub-genotype, minimal presence of norm genotypes, and excessive presence of het-norm/low sub-genotypes in BRCA1/2-positive women is that BRCA1/2 mutations are embryo-lethal gene mutations for humans. A low (CGG_{n<26}) sub-genotype allele, however, appears to block embryo lethality of BRCA1/2. Such low sub-genotypes can be present in het-norm/low, hom-low/low and hom/high/low FMR1 sub-genotypes, combined, representing approximately 25 percent of all women (see FIG. 17).

Het sub-genotypes have been reasonably well defined in their respective ovarian functions. They, somewhat surprisingly, in women with infertility were also associated with risk towards autoimmunity. Hom sub-genotypes, because of low prevalence, are, however, not yet defined. Because of small numbers, they, in this study, also had to be collapsed into a single group. Hom patients may, therefore, contain functionally opposing, and, therefore balancing, sub-genotypes but final determinations await further studies.

Like the FMR1 gene in its genotypes and sub-genotypes, BRCA1/2 may be associated with premature diminished ovarian reserve. The gene, however, has never before been suspected of being embryo-lethal in humans.

Some prior evidence for embryo-lethality of BRCA1/2 exists, however: When in the mid-1990 first BRCA1/2 mouse models were developed, heterozygous mice did not express expected phenotypes. Therefore, homozygous models were developed, which proved embryo-lethal. Different mouse mutant for BRCA1/2, however, display great variability in phenotypes and in rescue of embryonic lethality on a p53-null background.

BRCA1/2 genetically interacts with the p53 pathway, which is, at least partially, believed to explain the so-called “BRCA paradox,” with BRCA-deficient tumor cells rapidly proliferating, while BRCA-deficient embryos suffer from a proliferation defects. BRCA1 mutant embryos were partially rescued in a p53- or p21-deficient background, while BRCA2 knockout mice were less successfully rescued by absence of p53, suggesting that, in addition to p53, other factors contribute to the abnormal proliferation in BRCA1/2 deficient mouse embryos. In animal experiments, p53-nullizygousity, indeed, can rescue some BRCA1 mouse mutants but may only delay lethality.

Currently available data suggest that in humans the normal range for CGGₙ on the FMR1 gene is 26 to 34 repeats. The norm genotype of the FMR1 gene, with both alleles in normal range (CGGₙ₌₂₆₋₃₄), likely, represented the original (ur-) FMR1 gene. Mutations may have then generated the het and hom genotypes, depending on whether only one or both alleles mutated outside normal range.

Under such a concept, mutations of CGG_{n≧}34 generated high sub-genotypes through the gene's well-recognized ability to expand (12). The gene's so far largely uninvestigated ability to contract to CGG_{n<}26, resulted in low sub-genotypes, and this study demonstrates that it is responsible for the rescue of female embryos with BRCA1/2 mutations from lethality.

The here reported ability of the het-norm/low sub-genotype of FMR1 to help avoid BRCA1/2-associated embryo lethality to such a significant degree, adds to the rapidly increasing biological importance of the FMR1 gene. Until recently, FMR1 was, primarily, only known for neuro-psychiatric problems in association with traditional premutation and full mutation genotypes. The gene's only known not neuro-psychiatric disease association was, at premutation range (CGGₙ₌₅₅₋₂₀₀), with premature ovarian failure (POF), now often given the acronym primary ovarian insufficiency (POI). It was this association that led us to search for a potentially associated ovarian function effect of the FMR1 gene.

Aside from effects on ovarian reserve and, consequently, ovarian aging patterns, this led us to discover the association of the het-norm/low sub-genotype with an, at young age, rapidly follicle depleting polycystic ovary-like phenotype, and with substantially decreased pregnancy chances with infertility treatments.

Somewhat more surprising, we, however, also noted a close association of het-norm low with risk towards autoimmunity, while het-norm/high demonstrated strongly protective effects. This observation for the first time suggested opposing clinical effects of the two het sub-genotypes of the FMR1 gene, and did so outside of historically known neuro-psychiatric effects and newly discovered ovarian/fertility effects of the gene. Opposing clinical effects of het-norm/low and het-norm/high genotypes should, however, not surprise since CGGₙ₌₃₀, the median of the gene's normal range, has been described as switching point between positive and negative message.

Especially since autoimmunity demonstrates a disease preponderance in women over men, these findings significantly enhance the physiologic and clinical importance of the FMR1 gene. A potential autoimmune function of the FMR1 gene, however, dwarfs in comparison to what here reported data suggest: They imply that CGG_{n<}26, represented by het-norm/low, and possibly low hom sub-genotypes, biologically and clinically may be of far greater important than expanded CGG_{n>34} ranges, represented by het-norm/high and, possibly, hom-high genotypes. The latter, of course, include traditional premutation and full mutation genotypes of FMR1, the latter associated with the fragile X syndrome. Suffice it to say; low (CGG_{n<26}), in contrast to high CGGₙ counts (CGG_{n>36}), have so far, largely escaped attention, even though CGGₙ₌₃₀ has been described as switching point for the FMR1 gene's message.

The het-norm/low sub-genotype of FMR1, thus, successfully combats embryo lethality of BRCA1/2 mutations; the biological/clinical cost for survival, however, appears steep because it increases autoimmunity and BRCA1/2 mediated cancer risks.

FMR1, considering it maps to the 5′ untranslated exon 1 on the X chromosome at Xq27.3, a region now widely considered associated with autoimmune risks, appears to represent crossroads between autoimmunity and reproduction. It now appears that FMR1 is located at triple crossroads of autoimmunity, cancer and reproduction.

Lifetime risk for breast cancer in the U.S., according to most recent National Cancer Institute data, is 1 per 8.2 women for a 12.2 percent risk per woman. In presence of BRCA1/2 mutations, the risk increases approximately five-fold to ca. 60 percent. BRCA1/2 mutations, thus, account for 5-10 percent of all breast cancers.

Lifetime ovarian cancer risk in the U.S. is ca. 1.4 percent but this risk, in presence of BRCA1/2 mutations, is increased 10.7 to 28.6-fold to a 15 to 40 percent range. Overall, BRCA1/2, thus, accounts for 10 to 15 percent of all ovarian cancer risk.

While breast and ovarian cancers are the most frequent BRCA1/2 associated malignancies, other cancers also demonstrate increased prevalence. In association with BRCA1, those include, for example, malignancies of the uterine cervix and corpus, pancreas and colon; in association with BRCA2, they include, for example, cancers of pancreas, stomach, gallbladder, bile ducts and malignant melanoma.

Genetic cancer screening for BRCA1/2 mutations is now generally restricted to high risk families for breast and ovarian cancer. Avoidance of BRCA1/2 embryo lethality only in presence of the FMR1 sub-genotype het-norm/low, however, suggests that het-norm/low women, due to their substantial BRCA1/2 carrier status, should be at significantly increased risk for all BRCA1/2 associated cancers. In contrast, the reported association between BRCA1 and prematurely diminished ovarian reserve likely reflects the high het-norm/low prevalence in such patients.

As here again demonstrated, in excess of 20 percent of women in a general population can be expected to exhibit the het-norm/low sub-genotype, and ca. 25 percent, combined, a low (CGG_{n<26}) het or hom allele. These women, therefore, have to be considered at increased risk for BRCA 1/2-associated cancers, autoimmunity and infertility.

The estimated population frequency for BRCA1/2 mutations (0.024 to 0.04%) in recessive and polygenic models, respectively, causes 5 to 10 percent of all breast cancer risk and 10 to 15 percent of all ovarian cancer risk. Extrapolating, the het-norm/low FMR1 sub-genotype, representing approximately 78.8 percent of BRCA1/2 patients, spread over only ca. a quarter of all women, would reflect 3.95 to 7.9 percent of all breast and 7.9 to 11.9 percent of ovarian cancer risk, concentrated in only approximately a quarter of the female population. Therefore, approximately 75 percent of the female population could, thus, be assured of very low BRCA1/2 related cancer risks, and ca. 15 to 16 percent of women with het-norm/high sub-genotype of practically no risk at all. The impact from these findings on current breast cancer screening guidelines may, therefore, be significant.

Distributions of FMR1 genotypes and sub-genotypes as well as prevalence of BRCA1/2 mutations, of course, vary in different races/ethnicities. Interestingly, so do female cancers, autoimmunity and female infertility prevalence. These observations may be associated.

For women, the FMR1 gene, therefore, emerges as a potentially very important screening tool for risk towards a multitude of serious potential medical problems, including infertility, autoimmunity and cancer risks.

Based on the observation that BRCA in normal cells induces growth arrest, while promoting tumor formation in BRCA mutation carriers, some have pointed at the likely presence of secondary suppressor mutations, which may overcome BRCA-associated arrests during BRCA-associated tumorigenesis. With the FMR1 gene at crossroads of reproductive, immunologic and cancer-associated effects, it is tempting to hypothesize about just such, each other opposing, functions for the two het sub-genotypes of FMR1, het-norm/high and het-norm/low.

The here investigated patient populations are European and American, and, therefore, may reflect different genetic diversities. Furthermore, their retroactive evaluations may have resulted in selection biases. Assay performance in different laboratories may have resulted in divergent results between study groups. Similarities in FMR1 genotype and sub-genotype distribution between Austrian and U.S. control groups, however, practically rule out significant statistical impact from laboratory variability.

Statistical clarity of the here reported results, therefore, strongly supports reported assertions, which appear statistically robust. Combined, the results suggest major new biological and clinical importance for FMR1 and BRCA1/2 mutations.

### Example 11

### Objective of Example 11

Follicle maturation requires that the ovary converts from androgen dominance to an estrogen-dependent microenvironment. For decades androgens have, therefore, been considered detrimental to follicle maturation. More recently developed evidence, however, suggests that androgens may be essential to normal folliculogenesis and female fertility. At least in mice, these effects appear androgen receptor (AR)-mediated and granulosa cell-specific. Moreover, androgen activity appears to act synergistically with follicle stimulating hormone (FSH), though, possibly, antagonistic to anti-Müllerian hormone (AMH).

ur center introduced a number of years ago androgen, for example dehydroepiandrosterone (DHEA), supplementation into fertility treatment of women with diminished ovarian reserve (DOR). DHEA is effective in improving pregnancy chances and reducing miscarriage risks. The latter effect is at least in part the consequence of reduced embryo aneuploidy.

DHEA exerts fertility promoting effects. It is a mild androgen that gradually declines with advancing female age. Within the ovary, it serves as a precursor for the production of androstendione, testosterone and, consequently, estrogen. As an essential substrate for steroidogenesis, one can assume that DHEA deficiency, by itself, may be cause for impaired sustained follicular growth and development. DHEA supplementation, however, improves ovarian function, as assessed by AMH, in only selected women with DOR.

Why DHEA treatment is only effective in some women is unknown, and raises potentially important questions of whether differences in androgen metabolism may influence DHEA supplementation effects, and whether predictive phenotypical parameters can be developed in women with DOR for improvements of functional ovarian reserve (FOR) after DHEA supplementation.

Recent investigations suggest that how functional ovarian reserve of a woman declines with advancing age is, at least partially, dependent on ovarian genotypes and sub-genotypes of the FMR1 gene. Therefore, the potential androgen effects on follicle maturation may vary between different FMR1 genotypes and sub-genotypes. This study investigated in women, suffering from DOR, supplemented with DHEA, effects on androgen metabolism, stratified for FMR1 genotypes and sub-genotypes.

Methods and Materials of Example 11

This study investigated 91 consecutive infertility patients, undergoing first IVF cycles between February 2010 and July 2011, coded in our center's anonymized research data base as suffering from occult primary ovarian insufficiency (OPOI), at our center also given the acronym premature ovarian aging (POA).

A diagnosis of POA was established if women presented with abnormally elevated age-specific baseline follicle stimulating hormone (FSH) and/or abnormally low age-specific anti-Müllerian hormone (AMH) levels. Normal age-specific hormone levels were defined at all ages, as reported in detail.

Women diagnosed with POA at our center routinely receive supplementation with 25 mg of micronized DHEA, TID, for at least six weeks prior to initiation of IVF. DHEA levels, DHEA sulfate (DHEA-S), total testosterone (T) and free testosterone (free T) concentrations were measured longitudinally monthly on cycle days 2/3 from initiation of supplementation until IVF cycle start.

Again, in concordance with the center's routine protocol for POA patients, ovarian stimulation for IVF involved a microdose agonist protocol (leuprolide acetate, Lupron®, Abbott Laboratories, North Chicago, Ill.) and a daily gonadotropin dosage of 600 IU, with 3:1 FSH over human menopausal gonadotropin (hMG) preponderance (gonadotropin manufacturers varied, depending on patient preference). Oocyte retrieval was performed in women who presented with at least one follicle of at least 18 mm diameter.

All oocytes that were retrieved, independent of maturity and perceived quality, were considered in determining oocyte yields in representation of functional ovarian reserve. Clinical pregnancy was considered established after positive serum pregnancy test and observation of a gestational sac with fetal heart on vaginal ultrasound.

To assess effects of androgen metabolism, a series of logistic regression analyses was performed to determine whether total androgen concentrations, changes of androgen levels during DHEA treatment and androgen interactions affected pregnancy rates. Associations between (i) baseline androgen levels (i.e., androgen concentrations at DHEA start), (ii) intermediate androgen levels (i.e., androgen levels at monthly intervals during diagnostic work ups), and (iii) androgens at IVF cycle in association with pregnancy chance were analyzed. Where repetitive androgen analyses existed in the research database during lengthy work up periods, we utilized for pre-treatment assessments the first available blood draws that did not exceed two weeks after start of DHEA supplementation (median duration on DHEA 0.1 weeks); for the intermediate assessment we used blood draws that did not exceed an average of seven weeks from start of analysis (median 7.3 weeks), while the pre-IVF cycle draw was always the last blood draw prior to cycle start, with median DHEA supplementation of 13.4 weeks. In women, who had started DHEA supplementation prior to the study period, only androgen levels during work up periods and cycle starts were utilized for analysis.

Regression models were adjusted for potential confounding factors. Those included: female age, body mass index (BMI), FMR1 genotypes and sub-genotypes, duration of DHEA supplementation, total gonadotropin dosage used, number of oocytes retrieved and embryos transferred. Only factors that were significantly associated with pregnancy potential in nonparametric tests [Mann Whitney-U tests and chi square tests (χ²)] were, consequently, included in multivariate regression models (See Table 10 below).

TABLE 10
Patient characteristics and androgen profiles of 91 women with diminished
ovarian reserve, who underwent supplementation with DHEA and consecutive
in vitro fertilization.

| | | Clinical | |
|---|---|---|---|
| | Total | Pregnancy | Not Pregnant |
| | (n = 91) | (n = 23) | (n = 68) |
| Age (years) | 39.8 ± 4.4 | 38.3 ± 4.0 | 40.3 ± 4.5* |
| BMI (kg/m²) | 25.2 ± 5.5 | 24.4 ± 4.6 | 25.6 ± 5.8 |
| Race | % | % | % |
| African American n = 12 | 13 | 25 | 75 |
| Asian n = 10 | 11 | 40 | 60 |
| Caucasian n = 69 | 76 | 23 | 77 |
| Diagnosis | % | | |
| Male n = 21 | 23 | | |
| Endo n = 3 | 3 | | |
| PCO n = 7 | 8 | | |
| Diminished Ovarian Reserve | 50 | | |
| n = 45 | | | |
| Tubal n = 14 | 15 | | |
| Uterine n = 9 | 10 | | |
| FMR1 Genotype | | | |
| Normal n = 47 | 52 | 23 | 77 |
| Heterozygous/Homozygous | 48 | 28 | 72 |
| n = 43 | | | |
| FSH (mIU/mL) | 10.3 ± 4.5 | 10.2 ± 4.9 | 10.3 ± 4.4 |
| Baseline AMH (ng/mL) | 1.4 ± 1.9 | 1.5 ± 1.1 | 1.4 ± 2.1 |
| Estradiol (pg/mL) | 58.3 ± 32.3 | 55.6 ± 24.8 | 59.2 ± 34.6 |
| Gonadotropin dosage (IU) | 5617 ± 2236 | 5662.5 ± 1896.7 | 5603.4 ± 2341.5 |
| Oocytes at Retrieval | 7.9 ± 5.4 | 9.3 ± 5.1 | 7.4 ± 5.5* |
| Embryos Transferred | 2.5 ± 1.0 | 3.0 ± 0.9 | 2.3 ± 1.0* |
| Embryos Cryopreserved | 1.1 ± 2.5 | 1.5 ± 2.6 | 1.0 ± 2.5 |
| Baseline | | | |
| DHEA (ng/dl) | 435.4 ± 248.0 | 427.1 ± 202.7 | 440.3 ± 264.9 |
| DHEA-S (mcg/dl) | 190.6 ± 143.3 | 177.8 ± 148.1 | 194.8 ± 143.4 |
| Total Testosterone (ng/dl) | 20.6 ± 9.0 | 18.5 ± 7.0 | 20.8 ± 9.4 |
| Free Testosterone (ng/dl) | 1.7 ± 0.9 | 1.3 ± 0.8 | 1.8 ± 0.8 |
| Work Up | | | |
| DHEA (ng/dl) | 627.9 ± 299.1 | 850.1 ± 414.8 | 586.9 ± 259.6 |
| DHEA-S (mcg/dl) | 388.4 ± 162.3 | 421.4 ± 180.1 | 382.6 ± 160.8 |
| Total Testosterone (ng/dl) | 27.3 ± 16.0 | 22.0 ± 4.2 | 27.9 ± 16.8 |
| Free Testosterone (ng/dl) | 2.8 ± 1.7 | 2.8 ± 2.1 | 2.8 ± 1.7 |
| Cycle Start | | | |
| DHEA (ng/dl) | 611.3 ± 251.3 | 588.5 ± 266.5 | 618.8 ± 247.9 |
| DHEA-S (mcg/dl) | 373.8 ± 186.3 | 400.7 ± 220.8 | 365.0 ± 174.8 |
| Total Testosterone (ng/dl) | 27.6 ± 13.7 | 32.9 ± 11.5 | 25.5 ± 14.1* |
| Free Testosterone (ng/dl) | 2.9 ± 1.9 | 3.4 ± 1.3 | 2.7 ± 2.1* |

*p < 0.05

This included age, number of retrieved oocytes and embryos transferred. To alleviate potential confounding effects from variable DHEA supplementation periods, every step of the analysis was adjusted for DHEA supplementation length. Table 10 lacks p-values where factors were not associated with pregnancy. Those included gonadotropin dosage (p=0.95), number of cryopreserved embryos (p=0.50), baseline FSH (p=0.74), baseline estradiol (p=0.89), BMI (p=0.51). In addition, neither race/ethnicity nor FMR1 genotype and sub-genotype distribution were associated with clinical pregnancy.

FMR1 genotypes were assigned based on a normal range of 26-34 (median 30) CGG repeats: normal was defined by both alleles within range, while abnormal was defined by the presence of one or both alleles outside the normal range, thus including heterozygous and homozygous genotypes and all of their sub-genotypes, as previously reported.

Statistical analysis was undertaken using SPSS 18.0 (SPSS, Chicago, Ill.). Baseline characteristics of patients were compared using t-tests. Continuous values are presented as mean±SD. Continuous values are presented as mean±SD. Outcome parameters are presented as proportions. A P-value <0.05 was considered statistically significant.

Here presented data involved retrospective review of medical records and an anonymized electronic research database.

Results of Example 11

The study group presented with a mean age of 39.8±4.4 years; mean baseline FSH was 10.3±4.5 mIU/mL; and mean AMH was 1.4±1.9 ng/mL. Longitudinal androgen measurements showed the following mean concentrations (Table 10): (i) at baseline, (ii) during the work up and (iii) at cycle start: DHEA, 435.4±248.0 ng/dl, 627.9±299.1 ng/dl, and 611.3±251.3 ng/dl, respectively; DHEA-S, 190.6±143.3 mcg/dl, 388.4±162.3 mcg/dl and 373.8±186.3 mcg/dl, respectively; total testosterone, 20.6±9.0 ng/dl, 27.3±16.0 ng/dl and 27.6±13.7 ng/dl, respectively; and free testosterone, 1.7±0.9 ng/dl, 2.8±1.7 ng/dl and 2.9±1.9 ng/dl, respectively.

Table 10 also demonstrates that patients required a mean total gonadotropin dosage of 5617±2236 IU to produce a mean number of 7.9±5.4 oocytes. Out of 91 IVF patients with POA, 23 women (25.3%) achieved clinical pregnancy.

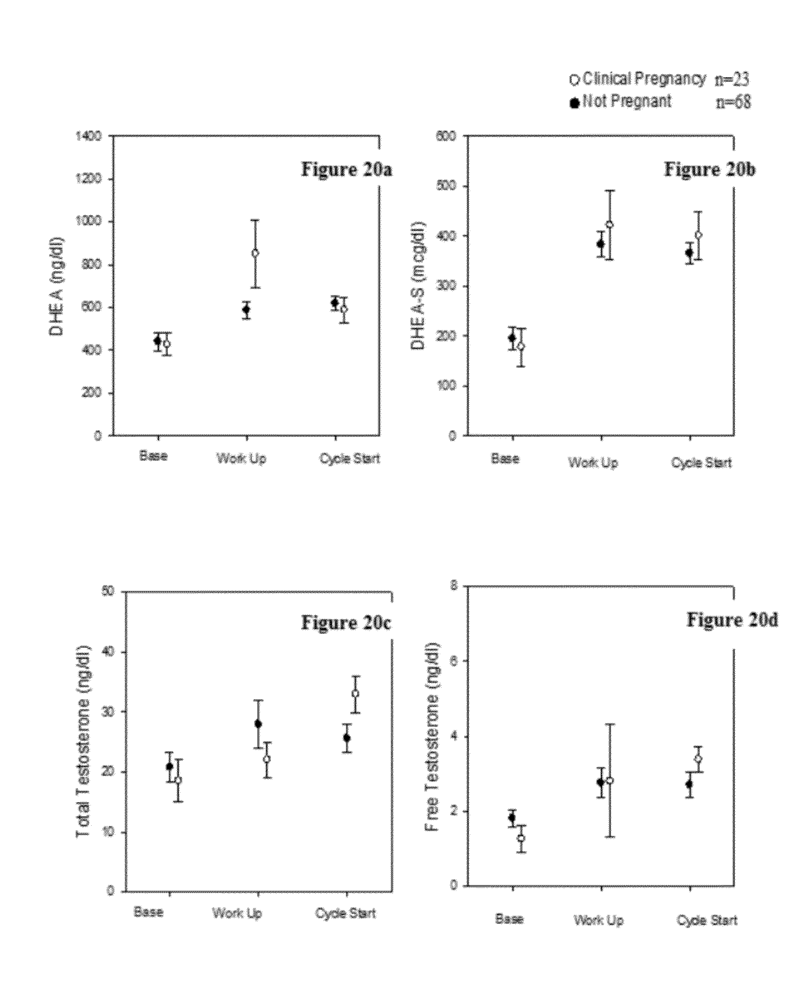

FIGS. 20 a-d show longitudinal androgen concentrations (monthly intervals) and pregnancy chances in 91 women with diminished ovarian reserve, who underwent DHEA supplementation and consecutive in vitro fertilization (IVF). Androgen concentration with asterisks are significantly different (Mann-Whitney U, p<0.05).

Adjusted for duration of DHEA supplementation, androgen concentrations at baseline and cycle start were, overall, not associated with pregnancy potential. However, DHEA concentrations during work up were significantly associated with higher pregnancy rates (β=0.275, SE±0.140, p=0.049). For every unit of increase in DHEA concentration, pregnancy potential increased 1.32-fold or by 123% (FIGS. 20 a-d). This association of increasing DHEA serum concentrations on pregnancy outcome did not remain significant when adjusted for duration of DHEA supplementation, age, and number of oocytes retrieved (FIGS. 20 a-d).

When the influence of individual androgen concentrations on pregnancy potential (corrected for DHEA supplementation duration) was evaluated, stratified for FMR1 genotype, DHEA-S, at cycle starts, almost reached significance in affecting clinical pregnancy potential in women with normal FMR1 genotype (β=0.577, SE±0.305, p=0.058). For every unit of increase in DHEA-S, pregnancy potential increased by a factor of 1.78 or 178 percent.

At cycle start, among women with abnormal FMR1 genotypes, total testosterone (β=0.257, SE±0.098, p=0.009), and free testosterone (β=1.199, SE±0.504, p=0.017) affected clinical pregnancy potential. For every unit increase in total and free T, pregnancy potential increased by factors of 1.29 and 3.32, respectively.

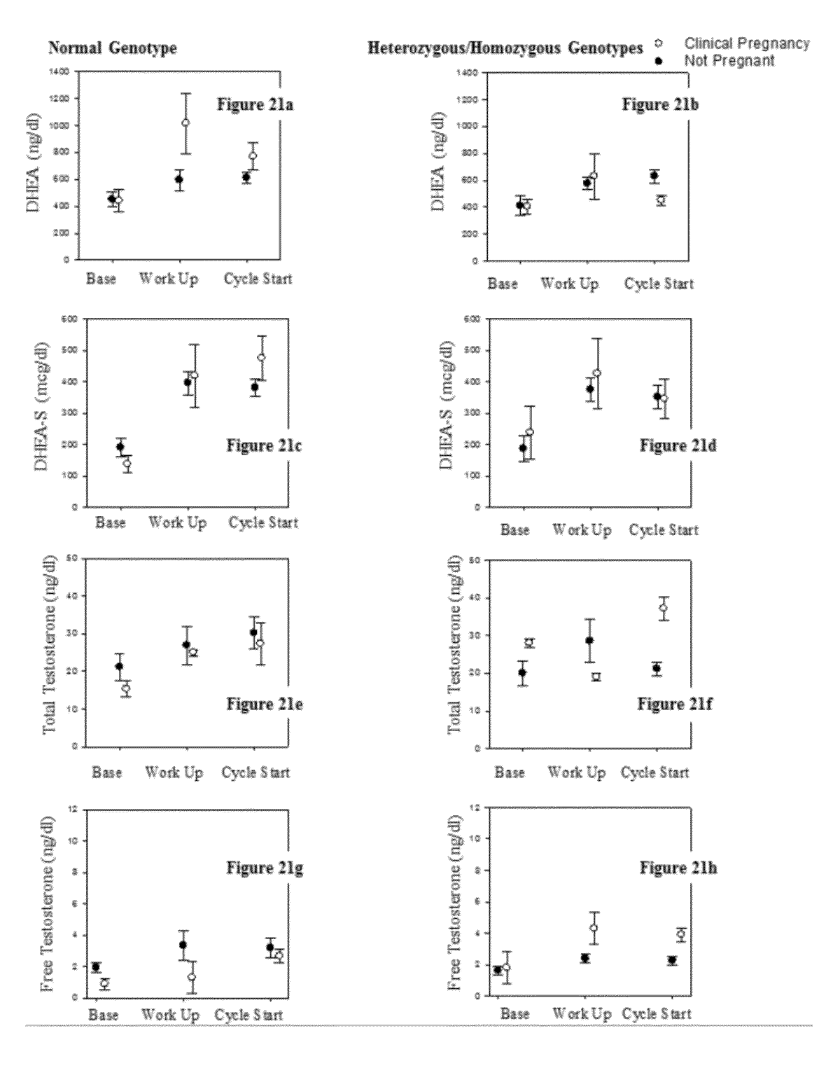

DHEA had only almost significant effect (β=−0.422, SE±0.233, p=0.059) on clinical pregnancy potential, with every unit increase of DHEA, pregnancy chances dropping by a factor of 0.66 (65.6%). When corrected for age, duration of DHEA supplementation, and oocyte yield, free testosterone significantly affected clinical pregnancy potential (β=1.101, SE±0.508, p=0.03), increasing pregnancy potential by a factor of 3.01 (301%). Both DHEA (β=−0.414, SE±0.227, p=0.068) and total testosterone (β=0.497, SE±0.268, p=0.064) almost reached statistical significance (FIGS. 21 a-h), resulting in respective decreases in pregnancy chances to 0.66 (66.1%) or by a factor of 1.51 for DHEA and an increase in pregnancy potential by a factor of 1.64 (164%) for T.

When the interaction of DHEA with DHEA-S, total and free testosterone, in regards to pregnancy potential was analyzed, corrected for duration of DHEA supplementation, the following results were obtained: Androgen interactions at baseline and during work up did not affect pregnancy rates; androgen interactions between DHEA levels and total T (β=−0.052, SE±0.021, P=0.012) and between DHEA and free T (β=−0.375, SE±0.153, p=0.014) at cycle start did, however, significantly affect pregnancy rates. This means that for every unit increase in interaction term between DHEA and total T the odds for clinical pregnancy declined to 0.949 (94.9%) or a factor of 1.05, with the change being due to DHEA levels, and not T, while interaction between DHEA and free T declined by 0.687 (68.7%) or a factor of 1.46.

When adjusted for length of DHEA supplementation in weeks, age, and oocyte yield, interactions between DHEA and total testosterone (β=−0.058, SE±0.023, p=0.010) and between DHEA and free testosterone (β=−0.496, SE±0.197, p=0.012) remained significant, meaning that for every unit increase in interaction term pregnancy chances declined by 9.944 (94.4%), a factor of 1.06, and 0.61 (60.8%), a factor of 1.64, respectively.

To assure that observed effects were the consequence of DHEA supplementation, we created an interaction term between total and free T as continuous variables and DHEA as a bivariate, with one state representing the normal physiologic state and the other anticipated DHEA levels in presence of supplementation. (i.e., levels above the upper limit of normal). This analysis confirmed the effects of DHEA supplementation by demonstrating that, in presence of supraphysiologic levels of DHEA, incremental increases of total and free T, independently, increased odds of pregnancy (total T by a factor of 1.18; p=0.012; free T by a factor of 6.65 (p=0.007).

Discussion of Example 11

The here reported results demonstrate that the androgen metabolism of a woman, indeed, significantly affects pregnancy rates in women with diminished ovarian reserve, previously supplemented with DHEA. Women unable to effectively metabolize DHEA experience significantly impaired pregnancy chances in comparison to those, who demonstrate quick rises in DHEA levels and good metabolization (FIG. 20). Those androgen-related effects on pregnancy potential were, however, only apparent at IVF cycle start, after weeks of DHEA supplementation. This latency in treatment effect points towards androgen effects at a relatively early stage of follicle development.

Such a timeline corresponds well to the androgen-receptiveness of small preantral and early antral follicles, observed in animal models. It has been demonstrated that growth-promoting effects of androgens in small growing follicles of androgen receptor knockout mice, which imply importance of androgens for normal follicle maturation and female fertility. As pre-antral follicles require several weeks to reach ovulation, treatments focusing on preantral- and early antral-stage follicles, of course, will only prove effective weeks after treatment initiation. These findings, therefore, correspond well to reported clinical observations with DHEA supplementation, where beneficial effects were only seen after at least six weeks of supplementation.

Delayed treatment effects may also explain why human data in regards to androgen supplementation have remained somewhat contradictory. For example, poorer IVF pregnancy rates in women with testosterone concentrations below 20 ng/dL at cycle start have been reported; in contrast, some reports have failed to show beneficial effects of short-term testosterone supplementation in poor responders.

If, as our results suggest, androgen metabolization is, indeed, of importance for proper follicle maturation, women with higher testosterone concentrations may experience better pregnancy chances in association with IVF. Analysis of the whole study cohort, however, does not demonstrate such an association. This, and earlier reported evidence of only selective effectiveness of DHEA supplementation, therefore, raise questions why androgen supplementation does not affect all patients with DOR identically.

FMR1 genotypes and sub-genotypes demonstrate distinct ovarian aging patterns and IVF pregnancy chances. We, therefore, in this study, in addition, investigated whether androgen effects in DHEA-supplemented DOR patients varied with underlying FMR1 genotypes and sub-genotypes.

Not completely surprising, androgen effects, indeed, varied depending on ovarian FMR1 genotypes: While the normal FMR1 genotype did not show an association between testosterone concentrations and pregnancy chances, women with abnormal (heterozygous and homozygous) FMR1 genotypes demonstrated significantly higher pregnancy potential with increased testosterone concentrations at cycle start. These results remained significant when controlled for confounding factors, such as female age, oocyte numbers and duration of DHEA supplementation.

FIGS. 21 a-h are longitudinal androgen concentrations (monthly intervals) and pregnancy chance in 91 women with diminished ovarian reserve, who underwent DHEA supplementation and consecutive in vitro fertilization (IVF) stratified for FMR1 genotypes. Androgen concentration with asterisks are significantly different (Mann-Whitney U, p<0.05).

As FIGS. 21 a-h demonstrate, women who conceived, started treatment with lower testosterone, but by IVF cycle start had reached higher levels than women who failed to establish pregnancy. These findings correspond to data that shows lower pregnancy rates in DOR patients with low serum testosterone levels. Testosterone concentrations, however, did not affect cycle outcomes in women with normal ovarian response and, therefore, likely normal DOR.

Here presented data in DOR patients, thus, confirm the importance of androgens for follicle maturation in humans. If left untreated, suboptimal levels of androgens appear clearly associated with lower pregnancy chances in IVF, though this association appears restricted to women with abnormal FMR1 (heterozygous and homozygous) ovarian genotypes. Indeed, since normal and abnormal FMR1 genotypes, each, represent approximately half of any female population, this observation may explain why an otherwise seemingly so obvious clinical association has been overlooked for so long.

Patients who conceived had the lowest androgen levels at presentation and the highest by time of IVF cycle start (FIG. 20). They, therefore, very obviously, have, possibly due to genetically determined deficiency of substrates for androgen production, the largest need for androgen supplementation. Once androgen deficiency is eliminated, their ability to metabolize DHEA into testosterone significantly improves pregnancy chances.

In contrast, pregnancy potential in women with normal FMR1 genotype does not appear related to absolute testosterone concentrations. One possible explanation is that the normal FMR1 genotype is inherently associated with higher testosterone/androgen levels than abnormal genotypes and sub-genotypes.

One may assume a minimum androgen threshold, and possible therapeutic range, for sustained follicle development that was only reached in women with normal FMR1 genotype. Such a range may be flexible, as we propose as a general principle for hormonal activities, and, because of synergism between androgens and follicle stimulating hormone (FSH) during early follicle development stages, could be clinically assessed in androgen/FSH ratios. One within such a context also can presume that too high androgen levels, above therapeutic range, may cause adverse effects on follicle maturation.

Our results also offer an alternative explanation: Absolute androgen deficiency may, simply, be a defining characteristic of DOR. Indeed, androgen deficiency may be genetically linked to abnormal FMR1 genotypes and sub-genotypes. Such an explanation may suggest varying phenotypical profiles in women with DOR, depending on FMR1 genotype, and a functional association between the FMR1 gene and androgen metabolism. Such an explanation would then suggest varying phenotypical profiles in women with DOR, depending on FMR1 genotype, and a functional association between the FMR1 gene and androgen metabolism.

Through early preantral stages, normal folliculogenesis is believed to require synergism between androgens and FSH to optimized follicle recruitment and growth. Treatment of rhesus monkeys with testosterone increased mRNA for the FSH receptor, while FSH injections, in turn, increased androgen receptor mRNA in primary follicles. The importance of interactions, rather than absolute androgen concentrations, at preantral stage suggests a balanced interplay of various factors as essential for follicular development. Both outlined possible mechanisms may, therefore, be at play in combination.

Synergistic interactions between androgens and FSH have been demonstrated by showing increased DHEA-S levels under ovarian hyperstimulation. This androgen-FSH related effect was attributed with increases in ovarian insulin-like growth factor (IGF-1). IGF-1, indeed, appears reduced in women with diminished ovarian reserve.

At that stage, FSH activity appears to necessitate modulating factors like activin, inhibin and IGF-1. IGF-1 appears to be particularly critical for this follicular developmental stage, since IGF-1 knock-out mice present with arrested follicles at late preantral stage, and do not respond to gonadotropins. The right ovarian “microenvironments,” therefore, appear essential for normal follicle maturation.

For instance, a group has demonstrated that mutations in the regulation pathways of folliculogenesis that alter the ovary's hormonal environment are associated with increased meiotic non-disjunction in mouse oocytes. This group also predicted that pharmacologically induced changes in ovarian environments might beneficially affect aneuploidy rates. Age-related decline in female fecundity is mainly attributed to increases in aneuploidy. If segregation errors during the oocyte's meiotic divisions are, indeed, mediated by the follicle's “microenvironment,” its deterioration (by age or underlying pathophysiology) will, likely, compromise the oocyte's chromosomal competence, and lower euploidy rates will reduce pregnancy potential.

Restoration of negatively affected “microenvironments” to better health, for example via DHEA supplementation, may, at least partially, reverse selective effects of the premature ovarian aging process. DHEA supplementation, for example, appears to reduce aneuploidy and, by doing so, not only increases pregnancy chances, but also reduces miscarriage risks. Women who are capable of establishing such a microenvironment may experience higher pregnancy chances. IVF patients with normal FMR1 genotype, indeed, may experience higher pregnancy rates when compared to their counterparts with abnormal FMR1 genotypes and sub-genotypes.

In conclusion, our results suggest an essential role for androgens in folliculogenesis and establishment of pregnancy in infertile women with DOR. DHEA supplementation appears to facilitate restoration of androgenic ovarian endocrine microenvironments, which positively influence pregnancy chances with IVF. Clinical effectiveness, however, varies with FMR1 genotypes. In a contrarian analogy to hyper- and normo-androgenic phenotypes of polycystic ovarian syndrome (PCOS), these findings, for the first time, establish hypo-androgenic and normo-androgenic phenotypes of DOR. Like hyper-androgenism is seen as a characteristic feature of PCOS, hypo-androgenism now has to be viewed as a typical feature of DOR, characterized by their ovarian FMR1 genotypes.

While the foregoing written description of the invention enables one of ordinary skill to make and use what is considered presently to be the best mode thereof, those of ordinary skill will understand and appreciate the existence of variations, combinations, and equivalents of the specific exemplary embodiments thereof. The invention is therefore to be limited not by the exemplary embodiments herein, but by all embodiments within the scope and spirit of the appended claims.

What is claimed is:

1. A method of screening a human for risk of malignancies, said method comprising:
isolating the human's FMR1 gene, wherein the FMR1 gene has a first allele and a second allele;
measuring the number of triple CGG repeats on each of the first and second alleles, wherein the measuring step is conducted through use of an assay;
identifying the human as at risk for cancer when the triple CGG repeat number for at least one of the first and second alleles is less than 26.

2. A method according to claim 1, wherein the identifying step further comprises when the triple CGG repeat number for at least one of the first and second alleles is between 26-34.

3. A method according to claim 1, wherein the identifying step further comprises when the triple CGG repeat number for each of the first and second alleles is less than 26.

4. A method according to claim 1, wherein the identifying step further comprises when the triple CGG repeat number for at least one of the first and second alleles is greater than 34.

5. A method according to claim 1, wherein the cancer includes at least one of breast cancer and ovarian cancer.

6. A method according to claim 1, wherein the human is female.

7. A method according to claim 1, wherein the assay is at least one of Southern blotting and polymerase chain reaction.

8. A method according to claim 1, further comprising conducting a secondary test by isolating the human's BRC1 gene and the BRC2 gene, analyzing each of the BRC1 and BRC2 genes for mutations by performing at least one of denaturation high performance liquid chromatography and chain-terminating inhibitors.

9. A method according to claim 1, further comprising confirming the presence of cancer by conducting a secondary test and analyzing the results of the secondary test.

10. A method according to claim 9, further comprising administering to the human at least one a gene blocker and a therapeutic drug to interfere with the cancer.

11. A method of screening a human female for the clinical effectiveness of an androgen in a human female, said method comprising:
measuring the female's testosterone levels;
administering an androgen to the female;
isolating the female's FMR1 gene, wherein the FMR1 gene has a first allele and a second allele;
measuring the number of triple CGG repeats on each of the first and second alleles, wherein the measuring step is conducted through use of an assay;
measuring the female's testosterone levels again;
identifying the androgen as clinically effective such that the female has an increased pregnancy potential when the female's testosterone levels were higher when measured again and when the triple CGG repeat number for at least one of the first and second alleles is less than 26 or greater than 34.

12. A method according to claim 12, wherein the androgen is DHEA.

13. A method of screening a human for increased embryo quality, said method comprising:
isolating at least one of the human's BRC1 and BRC2 genes;
analyzing each of the BRC1 and BRC2 genes for mutations, and when a mutation of at least one of the BRC1 and BRC2 gene is present, isolating the human's FMR1 gene, wherein the FMR1 gene has a first allele and a second allele;
measuring the number of triple CGG repeats on each of the first and second alleles, wherein the measuring step is conducted through use of an assay;
identifying increased embryo quality and increased embryo survival, when the triple CGG repeat number for at least one of the first and second alleles is less than 26.
